(19) United States
(12) Patent Application Publication
(10) Pub. No.: US 2014/0295415 A1
(43) Pub. Date: Oct. 2, 2014

(54) LOW COST, DISPOSABLE MOLECULAR DIAGNOSTIC DEVICES

(71) Applicant: Diagnostic For All, Inc., Cambridge, MA (US)

(72) Inventors: Jason Rolland, Belmont, MA (US); John Thomas Connelly, Somerville, MA (US)

(73) Assignee: DIAGNOSTICS FOR ALL, INC., Cambridge, MA (US)

(21) Appl. No.: 14/353,654

(22) PCT Filed: Nov. 2, 2012
(86) PCT No.: PCT/US2012/063190

§ 371 Date: Apr. 23, 2014

Related U.S. Application Data
(60) Provisional application No. 61/701,199, filed on Sep. 14, 2012.
(60) Provisional application No. 61/555,981, filed on Nov. 4, 2011.

(51) Int. Cl. C12Q 1/68

(52) U.S. Cl. CPC C12Q 1/68
USPC 435/6.1; 435/287.2

(57) ABSTRACT

The invention provides molecular diagnostic test devices and methods for using such diagnostic test devices to detect analytes of biological significance in a patient. The diagnostic test devices are particularly useful for detecting a polynucleotide analyte in a sample obtained from a patient. Further, the diagnostic test devices are inexpensive, disposable, easy to use, and are useful at the point of care.

### RELATED APPLICATIONS

This application claims the benefit of and priority to U.S. Provisional Patent Application Ser. No. 61/555,981, filed Nov. 4, 2011, and U.S. Provisional Patent Application Ser. No. 61/701,199, filed Sep. 14, 2012, the contents of each of which are hereby incorporated by reference.

### GOVERNMENT SUPPORT

This invention was made with support provided by the Defense Advanced Research Projects Agency (Grant No. HR0011-12-2-0010); therefore, the government has certain rights in the invention.

### FIELD OF THE INVENTION

The invention relates to molecular diagnostic test devices and methods for using such diagnostic test devices to detect analytes of biological significance in a patient.

### BACKGROUND

Analytical devices for detecting the presence of biological materials are important for the detection and diagnosis of medical disorders. Cheap, disposable analytical devices capable of detecting biologically significant analytes are particularly important for providing basic medical testing to patient populations without ready access to a hospital or other medical facilities with instrumentation for analytical analysis of biological samples. The present invention addresses the need for cheap, disposable analytical devices capable of detecting biologically significant analytes, particularly polynucleotide sequences, in a biological sample obtained from a patient.

To address the aforementioned need, one aspect of this invention relates to the design of molecular diagnostic tests, i.e., analyses of clinical significance based on genetic features of a patient, his neoplasia, or an organism infecting the patient. Another aspect of the invention relates to detecting the up or down regulation of specific polynucleotide sequences (i.e., mRNA) which relate to a physiological response such as trauma, shock, or infection. More particularly, the invention provides a family of such tests exploiting technology involving disposable devices made by formation of microfluidic flow channels within horizontally arranged or stacked (and therefore three dimensional), absorptive, flow sheet material, e.g., paper. Devices of the invention enable detection of polynucleotide sequences present in a human, animal, or plant subject or a pathogen infecting the subject from a blood, saliva, or other biological sample, where the presence of the polynucleotide sequence, e.g., a particular nucleotide sequence, repeat, single nucleotide polymorphism, or other feature, is diagnostically or prognostically informative. Devices described herein are useful at the point of care, require little or no external equipment, and enable determination of the presence or absence of one or more genetic characteristics through use of an inexpensive, disposable, easy to use, optically readable test device.

### SUMMARY

The invention provides molecular diagnostic test devices and methods for using such diagnostic test devices to detect analytes of biological significance in a patient. The diagnostic test devices are particularly useful for detecting a polynucleotide analyte in a sample obtained from a patient. Further, the diagnostic test devices are inexpensive, disposable, easy to use, and are useful at the point of care.

In accordance with the invention, to implement such tests, one executes in one integrated device the following three generic operations:
1) Isolation, capture, and processing of target DNA/RNA from a sample, e.g., a whole blood sample, using a paper-based device, preferably with as little as 1,000 copies/mL of the target sequence present in the sample.
2) Amplification of oligonucleotides on a paper-based device, preferably using an isothermal method to obtain on the order of 10⁶-10⁹ copies of the target.
3) Detection of an amplified oligonucleotide on or using the paper-based device.

Accordingly, in its broadest aspects, the invention provides a molecular diagnostic device for detecting a characteristic of a polynucleotide analyte present in a sample. By the phrase “characteristic of a polynucleotide” we mean the presence or absence of a particular feature of the polynucleotide, such as a particular sequence, a particular base, a repeat, a deletion, a transposition or the like, the presence (or absence) of which in the sample provides some diagnostic or prognostic information, permits selection of a therapy, or is otherwise informative of the present or future health or condition of a patient. This is done by detecting the presence of or quantifying the amount of polynucleotide amplicons that will be generated in the device only in the presence of a detectable number of copies of a specific nucleotide sequence in the sample. The device comprises at least first, second, and third substantially planar members, one or more of which comprises fluid-impermeable barriers which define boundaries of hydrophilic regions therein which support fluid flow across and therethrough, and are designed to execute various functions as disclosed herein. One of the members defines a test zone for collection, amplification, and potentially visualization of a sample. At least one of the members is moveable relative to the others so as to permit establishment of fluid flow communication serially with the test zone and at least two or more hydrophilic regions, and to permit interaction with reagents disposed in fluid communication with the test zone at first and second stations, and optionally additional stations. The first station comprises a polynucleotide capture region wherein the test zone is disposed in fluid communication with a sample inlet; a second station comprises a polynucleotide amplification region wherein the test zone and captured polynucleotides are in fluid communication with a buffer inlet and dried amplification reagents. The device may additionally comprise a third station comprising an optically observable detection readout wherein the test zone and captured polynucleotides are in fluid communication with a buffer inlet and one or more polynucleotide detection reagents. The device may comprise additional stations, or plural regions within a generic station, to facilitate processes including, but not limited to, application of a wash agent or thermal nucleotide amplification.

Each of the stations may comprise a single station, as exemplified herein, or one or more substations, in which separate treatments of the sample or the polynucleotides or oligonucleotides present in the test zone are done separately. In the first station, the test zone is in fluid communication with one or more of a cell membrane or protein coat lysing reagent, a filter for removing particulates, a polynucleotide restriction reagent, a downstream liquid reservoir for drawing liquid, e.g., vertically, from the inlet to and through the test zone, and/or a surface which passively adsorbs polynucleotides. In this first station, nucleotides are isolated and captured so as to deliver to the second station an amplifiable polynucleotide product in the test zone, if analyte is present in the sample.

A second station may comprise a polynucleotide wash region supplementing or substituting an optional wash region in the first station, and a substation which comprises amplification reagents for conducting amplification, preferably isothermal amplification, disposed for uptake by buffer and in fluid communication with the test zone and the buffer inlet. It may further comprise at the same locus or a different substation a heater and insulation for maintaining the amplification region at a predetermined elevated temperature. In some embodiments the second station may further comprise a wash reservoir for drawing a wash fluid through the test zone.

In some embodiments, a third station comprises a polynucleotide detection reagent, preferably in a dried form, that can be placed in fluid communication with the test zone by lateral movement of the planar member comprising the test zone. In some embodiments a third station may comprise a wash reservoir for drawing a wash fluid through the test zone.

In some embodiments, the second station comprises an inlet and wash reservoir for drawing a wash fluid through the test zone, the third station comprises amplification reagents for conducting isothermal amplification, and a fourth station comprises a polynucleotide detection reagent. In yet another embodiment of the device, a first station comprises a sample inlet for sample application and nucleotide capture, a second station comprises a wash buffer inlet and wash reservoir for drawing a wash fluid through the test zone, a third station comprises amplification reagents for conducting isothermal amplification, a fourth station comprises a hermetically sealable chamber at which nucleotide amplification can be performed and evaporation is inhibited, and a fifth station comprises a polynucleotide detection reagent. In all embodiments, each station and its components can be brought into fluid communication with the test zone by stepwise, preferably unidirectional lateral movement of the planar member comprising the test zone relative to the other planar members. It will be apparent to one skilled in the relevant art in view of this specification that multiple devices can be conceived by rearranging the various elements described above and herein within the sliding planar member device described above.

The detection reagent preferably functions to develop color as the readout to indicate the presence or amount of amplified polynucleotide within or released from the test zone. Preferably, the detection reagents comprise a colored particle conjugate, or a labeled antibody or oligonucleotide probe reagent, labeled with an enzyme, a fluorophore, or a colored particle, for example, to permit colorimetric assessment of the presence of amplicons in the test zone. In some embodiments, the detection reagent may comprise an optically detectable DNA intercalating agent that can be detected via capture of fluorescence emission data. In some embodiments, detection may be facilitated via use of an amplification primer or a probe comprised of a nucleotide sequence complementary to the amplified polynucleotide product. In such embodiments, the primer or probe incorporates an antigenic tag (e.g., biotin, dinitrophenyl, or a fluoroscein) that can be recognized by a labeled antibody detection reagent. In such embodiments, detection of the polynucleotide amplicon may be achieved via binding of a labeled antibody reagent with affinity for the polynucleotide amplicon or probe bound to the amplicon.

In some embodiments, amplicons may be released from the test zone and drawn to a dwell region to facilitate antibody binding, and antibody-bound amplicons may be captured in a capture layer by another surface-bound antibody with affinity for the polynucleotide amplification product, a probe bound to the amplicon, or another antigenic moiety incorporated or directly or indirectly bound to the amplicon. In some embodiments, the capture region may be comprised of surface-bound capture oligonucleotides themselves comprised of nucleotide sequence complementary to the amplified polynucleotide product such that introduction of the amplified polynucleotide product to the capture region allows binding and capture of said amplification products by the surface-bound oligonucleotides. In some embodiments, the capture region may be comprised of surface-bound reagents that react with an enzyme or substrate integrated into the amplified product or coupled to an antibody that binds to the amplified product.

The detection reagent may be situated within the planar member above the test zone in a dried form such that movement of the sliding planar member comprising the test zone and addition of buffer results in establishment of fluid communication between the test zone and the station wherein the dried detection reagent is deposited, permitting dispersal of the dried detection reagent within the test zone. In some embodiments, the detection reagent may be added directly to the test zone following removal of the planar member containing the test zone from the planar assembly. The detection reagents and possibly the amplification reagents may be added directly to the region containing captured or amplified polynucleotides of the planar member comprising the test zone, following removal of said planar member from the device ensemble.

The device may incorporate elements that facilitate quantitative determination of the level of polynucleotide amplification. Such elements may include a colorimetric test readout, a negative control that upon absorption of the sample maintains or displays a predetermined color, and a positive control. Colorimetric assessment of polynucleotide amplification can be achieved by embedding in the device, e.g., in a reagent reservoir in fluid communication with the test zone, at least one dried, color-producing reagent arranged to produce a shade or pattern of color in a readout as a function of the concentration of an analyte in the sample. Also disposed in the device or manually added to the device is a dried, color-producing reagent which reacts at the positive control to produce color. In such devices, a valid test is indicated by a color change in the positive control and maintenance or display of a predetermined color at the negative control. In such an embodiment, polynucleotide amplification products can be released from the test zone to allow interaction at a site comprising the readout, negative control, and positive control zones. Establishment of fluid communication between the readout zone and the test zone can result in wicking of indicator species (e.g., magnesium ions) from the test zone to the readout zone, facilitating amplification readout based on the interaction of the detection reagent with said indicator species.

In another aspect, the invention provides a device for quantitative determination of a nucleotide analyte which has elements in common with the embodiments described above, but the colorimetric test readout includes a region of a color backing the readout, e.g., a region of printed color, which optically interacts with color developed at the readout to improve visual discrimination among different analyte concentrations in an applied sample. Thus, this type of device comprises porous, multiple hydrophilic sheets comprising plural functional regions including, but not restricted to, a liquid sample input; a colorimetric test readout including the region of a color backing the readout which optically interacts with color developed at the readout; and a colorimetric control. Disposed in the device is a dried, color-producing reagent responsive to interaction with a liquid sample derived from the test zone. The reagent is entrained and reacts with an analyte, if present in the applied sample, to produce a visually detectable change of color (as opposed to an intensity of a single color) in the readout as a function of the concentration of an analyte in the sample.

Devices of the invention may further comprise a color chart relating color at the readout to analyte concentration, and this may optionally be integrated with a sheet. Of course, plural readouts serviced by respective different dried, color-producing reagents are enabled by the disclosure herein.

The color producing reagent may react with any desired analyte, and in one preferred embodiment, reacts with polynucleotide amplification products. The negative control may comprise a colored area applied to a sheet which has an appearance when wetted different from when dry. The readout may comprise an area of the sliding planar member or another sheet that can establish liquid communication with the test zone. The readout may comprise immobilized binder which captures a colored species produced by the color-producing reagents. This permits display or a readout of the concentration of analyte in a sample as a portion of the area that develops color responsive to application of or interaction with liquid. The area may be continuous so that the concentration of analyte in a said sample is indicated, as in a mercury thermometer, by the linear extent of color development in the area. This is accomplished by providing capture reagents for the amplification products along a channel in combination with a colorimetric indicator. Higher quantities of amplification products will lead to color formation farther down the channel. Alternatively, the area comprises a plurality of separate areas and the concentration of analyte in the sample is indicated by the number of areas that develop color.

In further embodiments, the device further comprises a region defining a timer comprising a reservoir disposed in the device in liquid communication with the test zone which, after registering the test zone in a position that establishes liquid communication with the timer region, receives liquid from the test zone over a predetermined time interval and comprises indicia that the reservoir is filled and the device is ready to be read. The timer may for example comprise a channel of predefined dimensions which determines the length of time that liquid takes to reach the reservoir and to activate the indicia, which may comprise a printed message visible when the device is ready to be read. The timer also may function as a positive colorimetric control. Often, the timer is disposed downstream from the readout.

In further embodiments, the device further comprises downstream of the color-producing reagent and upstream of the colorimetric test readout, a dwell region which transports therethrough a mixture of analyte from a sample and the color-producing reagent, the dwell region comprising a multiplicity of micro flow paths including hydrophobic flow impeding surfaces, the numbers and dimensions of the micropaths serving to set the incubation time within a predetermined time interval as the mixture passes therethrough. The dwell region may be, for example, impregnated with a hydrophobic material (e.g., wax) which impedes the rate of liquid passage through the dwell region. In some cases, the dwell region is manufactured by printing a hydrophobic material onto a surface of a sheet and heating to absorb the hydrophobic material into the pores of the sheet.

In some embodiments, the device may comprise an adsorptive reservoir downstream of or stationed in a layer directly below and in fluid communication with the colorimetric test readout for drawing liquid along the flow path and through the dwell region and colorimetric test readout thereby to remove unbound colored species from the colorimetric test readout. A device may comprise in some instances an immobilized binder (e.g., an antibody) at the colorimetric test readout for capturing a complex formed during incubation in the dwell region. The device may include a sheet holding a dried, color-producing reagent in fluid communication with a parallel disposed sheet defining the dwell region. In certain embodiments, at least two of the following elements of the device are defined on a single adsorptive sheet: a region holding a dried, color-producing reagent; a reagent inlet; a colorimetric test readout; a dwell region; and an adsorptive reservoir.

In many embodiments, it will be necessary to release amplified polynucleotide products from the test zone for interaction with reagents located in another region of the device such as a readout zone or a dwell region. In such embodiments, it will be necessary to effect release of polynucleotide reagents from the test zone membrane either by addition of an appropriate release buffer to the test zone via an inlet or by bringing the test zone into fluid communication with a dried reagent capable of effecting polynucleotide release from the membrane comprising the test zone. Commercially available buffers or buffers known in the art to effect polynucleotide release from paper membranes such as Tris-HCl/EDTA (TE) can be employed to achieve release of amplification products from the test zone membrane.

In other embodiments, the device includes a washing reagent in fluid communication with polynucleotides captured or amplified in the test zone, which washing reagent functions to separate unbound species therein from said captured polynucleotides; establishment of fluid flow communication between a hydrophilic region and captured oligonucleotides is effected by movement of the members holding the test zone relative to the other members to register the test zone horizontally (i.e., in the same plane), or in some embodiments vertically (i.e., stacked in parallel planes), with the respective stations defined by the respective hydrophilic regions.

Certain components of the device comprise sheet-like material such as paper, cloth, or polymer film, such as nitrocellulose or cellulose acetate. The various function regions are defined by fluid-impermeable barriers that define boundaries of the plural hydrophilic regions. These are produced by screening, stamping, printing or photolithography and comprise a photoresist, a wax, poly(methylmethacrylate), an acrylate polymer, polystyrene, polyethylene, polyvinylchloride, a fluoropolymer, or a photo-polymerizable polymer that forms a hydrophobic polymer. The devices typically also comprises a fluid-impermeable layer disposed between adjacent members which layer defines openings permitting fluid flow from one member to another. The devices may comprise a patterned layer of adhesive which constitutes the barrier layer between adjacent adsorptive or absorptive sheets and which defines an opening permitting liquid flow communication between the sheets. Adsorbent layers or reservoirs may be exploited to advantage for drawing fluid from or through a hydrophilic region and through the test zone.

The devices permit multiplexing, i.e., simultaneously detecting a plurality of analytes. Processes for fabricating various functioning elements are outlined herein and disclosed in detail in international patent application publications such as WO/2008/049083, WO/2009/120963, WO/2009/121037, WO/2009/121041, WO/2010/102294, WO/2010/022324, and WO/2011/097412. The low cost of raw materials and facile, automated, multiplexed manufacture of such devices permits them to be made and sold at low cost, and used by relatively untrained persons

In another aspect, the invention provides an assay method comprising providing the device described above, adding a sample to the sample inlet, moving one member in relation to another to establish serially fluid communication between the test zone and the respective hydrophilic zones to permit fluid flow therebetween for a time interval and to execute multiple steps of an assay, and examining the test zone to determine the presence or absence of the analyte. In one embodiment, the method comprises providing the device described above, adding a sample to the inlet to capture oligonucleotides at the test zone, and moving one member in relation to another to establish fluid communication between the test zone, now containing captured oligonucleotides, and amplification reagents in the second station. This enables fluid flow therebetween for a time interval so as to amplify target oligonucleotide analyte, if present. Heat can be applied to the test zone containing captured oligonucleotides and released amplification reagents from an internal or external source in order to facilitate nucleotide amplification. Next, moving the member again in relation to the other members to establish fluid communication between the test zone, now containing captured and amplified oligonucleotides, and a third station containing a detection reagent to permit fluid flow therebetween for a time interval so as to visualize the presence of amplicons, if present, in the test zone, thereby to determine the presence or absence of a said analyte.

In another embodiment, the method comprises providing the device described above, adding a sample to the inlet to capture oligonucleotides at the test zone, and moving one member in relation to another to establish fluid communication between the test zone, now containing captured oligonucleotides, and a buffer wash in the second station. The buffer serves to remove unwanted material present in the sample while leaving the captured oligonucleotides adsorbed. Next, a member is moved to establish fluid communication between the test zone, now containing and purified captured oligonucleotides, and amplification reagents in the second station. This enables fluid flow therebetween for a time interval so as to promote exposure of the amplification reagents to the target oligonucleotide analyte, if present. Next, the device is heated to facilitate target oligonucleotide analyte amplification by said amplification reagents. After heating, the device may be maintained at an optimal temperature for a time period chosen to promote efficient and specific target oligonucleotide analyte amplification, and the planar member containing the test zone can be dried by heat. A detection reagent can then be added directly to the test zone so as to visualize the presence of amplicons, if present, in the test zone, thereby to determine the presence or absence of a said analyte and, in some embodiments, to quantify oligonucleotide amplification.

In yet another embodiment, the method comprises providing the device described above and adding a sample to the first inlet to capture oligonucleotides at the test zone. The method additionally comprises the step of moving one member in relation to another to establish fluid communication between the test zone, now containing captured oligonucleotides, and a second station comprising a buffer inlet positioned above the test zone and a wash reservoir positioned below the test zone for drawing a wash fluid therethrough. The movement of the sliding planar member to register the test zone at the second station brings the test zone into contact with a wash buffer, the buffer being placed at the second station either prior to or after fluid communication is established between the test zone and the second station. Establishment of fluid communication between the test zone and the second station allows the buffer to pass through the test zone and carry debris and free non-adsorbed nucleotides and polynucleotides away from the test zone to a wash reservoir. Further movement along the same lateral trajectory brings the planar member into contact with a third station comprising nucleotide amplification reagents. This enables fluid flow therebetween for a time interval so as to promote exposure of the amplification reagents to the target oligonucleotide analyte, if present. Next, the device is heated to facilitate target oligonucleotide analyte amplification by the amplification reagents.

Following this amplification step, the planar member can be moved further along the same lateral trajectory, to establish fluid communication between the test zone containing the products of the polynucleotide amplification reaction and a fourth station comprising a dried detection reagent. Establishment of fluid communication between the test zone and the dried detection reagent permits dispersal of the detection reagent within the test zone. In some embodiments, a liquid detection reagent can be added directly to the test zone via an inlet positioned at the fourth station. Interaction of the detection reagent with the test zone permits visualization of the presence of amplicons, if present, in the test zone, thereby to determine the presence or absence of a said analyte and, in some embodiments, to quantify oligonucleotide amplification. In other embodiments, after amplification is complete the test zone is analyzed for the presence of amplicons in a separate device. In other embodiments, the test zone may be separated from the remainder of the assembly before being subjected to analysis in a separate device, e.g., a spectrophotometer.

In yet another embodiment, the method comprises providing the device described above and adding a sample to the first inlet to capture oligonucleotides at the test zone. The method additionally comprises the step of moving one member in relation to another to establish fluid communication between the test zone, now containing captured oligonucleotides, and a second station comprising a buffer inlet positioned above the test zone and a wash reservoir positioned below the test zone for drawing a wash fluid therethrough. The movement of the sliding planar member to register the test zone at the second station brings the test zone into contact with a wash buffer, the buffer being placed at the second station either prior to or after fluid communication is established between the test zone and the second station. Establishment of fluid communication between the test zone and the second station allows the buffer to pass through the test zone and carry debris and free non-adsorbed nucleotides and polynucleotides away from the test zone to the wash reservoir. Further movement along the same lateral trajectory brings the planar member into contact with a third station comprising dried nucleotide amplification reagents. This enables fluid flow therebetween for a time interval so as to promote exposure of the amplification reagents to the target oligonucleotide analyte, if present.

The planar member is then displaced further along the same trajectory such that it registers at a fourth station comprising a hermetically sealable chamber suitable for nucleotide amplification. A sealing agent is dispersed within the device in such a way that unidirectional movement of the test zone into a position of contact with the fourth station and past the fourth station results in creation and destruction, respectively, of a hermetically sealed chamber that encompasses the test zone. While the test zone is sealed within the chamber comprising the fourth station, heat is applied to facilitate target oligonucleotide analyte amplification by said amplification reagents. Following this amplification step, the planar member can be moved further along the same lateral trajectory, to unseal the amplification chamber and establish fluid communication between the test zone containing the products of the polynucleotide amplification reaction and a fifth station comprising a dried detection reagent. Establishment of fluid communication between the test zone and the dried detection reagent permits dispersal of the detection reagent within the test zone. In some embodiments, a liquid detection reagent can be added directly to the test zone via an inlet positioned at the fifth station. Interaction of the detection reagent with the test zone permits visualization of the presence of amplicons, if present, in the test zone, thereby to determine the presence or absence of a said analyte and, in some embodiments, to quantify oligonucleotide amplification.

In yet another embodiment, the method comprises providing the device described above and adding a sample to the first inlet to capture oligonucleotides at the test zone. The method additionally comprises the step of moving one member in relation to another to establish fluid communication between the test zone, now containing captured oligonucleotides, and a second station comprising a buffer inlet positioned above the test zone and a wash reservoir positioned below the test zone for drawing a wash fluid therethrough. Movement of the sliding planar member to register the test zone at the second station brings the test zone into contact with a wash buffer. Establishment of fluid communication between the test zone and the second station allows the buffer to pass through the test zone and carry debris and free non-adsorbed nucleotides and polynucleotides away from the test zone to the wash reservoir. Further movement along the same lateral trajectory brings the planar member into contact with a third station comprising dried nucleotide amplification reagents. This enables fluid flow therebetween for a time interval so as to promote exposure of the amplification reagents to the target oligonucleotide analyte, if present.

The nucleotide amplification reagents may include primers that incorporate antigenic tags recognizable by an antibody disposed in another station of the device. Amplification via said tagged primers results in a polynucleotide amplification product that itself incorporates the antigenic tag. Following release of the dried polynucleotide amplification reagents to the test zone, the planar member is displaced further along the same trajectory such that it registers at a fourth station comprising a hermetically sealable chamber suitable for nucleotide amplification. While the test zone is sealed within the chamber comprising the fourth station, heat is applied to facilitate target oligonucleotide analyte amplification by said amplification reagents. Following this amplification step, the planar member can be moved further along the same lateral trajectory, to unseal the amplification chamber and establish fluid communication between the test zone containing the products of the polynucleotide amplification reaction and a fifth station comprising a dried detectable antibody-conjugated particle comprising a detection reagent and either a dried buffer reagent capable of effecting release of amplification products from the test zone or a buffer inlet that permits addition of a liquid buffer capable of effecting release of amplification products from the test zone.

Addition of release buffer or establishment of fluid communication with the dried buffer and antibody-coupled detection reagent results in release of the polynucleotide amplification product from the test zone membrane. The antibody-coupled detection reagent and the polynucleotide amplification product may then be drawn via an inlet into a dwell region located at the same station but in a planar member located below the test zone to facilitate binding of the antibody to the amplicon. Movement of the antibody-coupled reagent and amplification product through the dwell region brings them to a readout zone comprising at least a region containing surface-bound antibodies capable of binding the amplification product while it is bound to the antibody-coupled detection reagent. Additionally, a reservoir capable of drawing unbound antibody-coupled detection reagent may be located below the readout zone. In some embodiments, the readout zone is comprised of surface-bound capture oligonucleotides with sequence complementarity to the amplified nucleotide products rather than surface-bound antibodies.

In a further embodiment, an additional station situated between the fourth and fifth stations of the invention described above contains dried nucleotide probes with sequence complementarity to the amplified polynucleotides, sufficient to allow binding to a complementary DNA strand of the amplification product. The probes additionally incorporate an element capable of being recognized and bound by the dried and surface-bound antibodies found in the fifth station of the device described in the paragraph above. Movement of the test zone to this intermediate station permits fluid communication between the test zone and this station, allowing release of the dried nucleotide probes and binding to complementary regions of the polynucleotide amplification products. Movement of the sliding member to a fifth station permits release, capture, and detection of the polynucleotide amplification product in the manner previously described with the exception that the amplification products do not integrate primers comprising tags detectable by the antibodies of the fifth station and the antibodies found in the fifth station display antigenicity toward the tags of the nucleotide probes described above.

In still another aspect, the invention provides methods of manufacturing test devices for determination of one or more analytes in liquid biological samples enabling mass production of reliable, extremely inexpensive test devices designed for quantitative or semi-quantitative clinical assays for any one or combination of analytes. In one embodiment, the method of manufacturing comprises applying by printing onto a region of the surface of a sheet a predetermined density of ink, causing the ink to penetrate the sheet, and hardening the ink to form a dwell region comprising a multiplicity of micro flow paths including hydrophobic flow impeding surfaces defined by the ink, the numbers and dimensions of the micropaths serving to set a predetermined time interval for liquid sample to pass through the dwell region. The method may include the additional step of laminating the sheet to at least one additional porous, hydrophilic sheet which supports absorptive flow transport, at least a portion of which is in liquid communication with the sheet and which additional sheet defines at least one element selected from the group consisting of a flow path; a colorimetric test readout; an immobilized binder at a test region for capturing a complex; a second dwell region; a liquid reagent inlet; a control site; a dried, color-producing reagent reservoir, an adsorptive reservoir, and a sample split layer. A sample split layer allows a sample to be divided, for example, so that multiple assays can be run in parallel.

### BRIEF DESCRIPTION OF THE FIGURES

FIG. 1A shows an exploded view of a sliding strip device comprising five planar members showing movement of the sliding member comprising the test zone as it registers at different stations to facilitate steps of A) sample isolation, B) oligonucleotide amplification, and C) amplified oligonucleotide product detection;

FIG. 1B shows an exploded view of a sliding strip device comprising three planar members and five stations A) illustrating movement of the sliding member comprising the test zone as it registers at different stations while the following steps occur: B) sample application at a first station, C) nucleotide absorbance and debris filtration to a collection reservoir, D) application of a wash buffer, E) contact with the wash buffer and collection of the wash in a reservoir, F) contact with nucleotide amplification reagents at a third station, G) nucleotide amplification at a fourth station comprising a hermetically sealed chamber, and H) contact with nucleotide detection reagents at a fifth station;

FIG. 2 shows an exploded view of a sliding strip device used to demonstrate strip-based sample preparation and purification methods;

FIG. 3 illustrates A) Xylenol Orange and B) Eriochrome Black T, two colorimetric dyes that can be used to determine whether or not an amplification reaction has occurred via LAMP; where for Xylenol Orange, a negative result is indicated by a red color, and a positive result is indicted by an orange color; and for Eriochrome Black T, a negative result is indicated by a purple color, and a positive result is indicated by a blue color;

FIG. 4 shows a detection system used to obtain PI emission data, the system being comprised of an Ocean Optics USB4000-FL spectrofluorometer used in conjunction with a specially designed fluorescence/reflection fiber optic probe, pulsed Xenon lamp, filters, and probe holder;

FIG. 5 illustrates steps involved in capturing an antigen in a multilayered planar device employing an antibody-coupled detection reagent and a surface-bound antibody stationed in a capture layer;

FIG. 6 illustrates how a colored ink can be used to enhance the contrast of a colorimetric readout, for instance, where a yellow ring of colored wax ink is used to enhance a blue signal output;

FIG. 7 shows a control region of a device that undergoes a color change from white to yellow when wet;

FIG. 8 illustrates a device incorporating a timing element into a control region;

FIG. 9 shows a comparison of color readout on a white background (top panel) and a yellow background (bottom panel) illustrating improved contrast with the yellow background;

FIG. 10 shows a device configured for quantitative colorimetric readout; more filled circles means higher concentration of analyte;

FIG. 11 is a plan and perspective view that shows a device for quantitative colorimetric readout that includes a color chart for automated calibration;

FIG. 12 illustrates a design and methodology for using a multiplexed sliding strip device wherein: A) an exploded view of the device reveals four layers comprising four planar members with the uppermost layer serving as the sample receiving layer, the layer directly below serving to both split the sample into separate channels as well as store reagents for amplification and detection, the next lowest layer comprising a sliding member containing two sample discs (test zones), and the bottommost layer containing wash channels, B) a sample is introduced into the first station where it is filtered, divided into two separate paths, processed for lysis and nucleotide adsorption, and wicked through to the reservoir, C) the sliding member is brought into contact with the second station and wash buffer is introduced to the second station, D) the strip is then slid to a third station where the discs dissolve dried amplification reagents, E) the strip is slid to a fourth station where the discs are hermetically sealed and heating/amplification occurs, and F) the strip is slid to a fifth station where the discs contact a colorimetric reagent stored in the device;

FIG. 13 illustrates A) a device that consists of three substantially planar members that lay within the same horizontal plane, operated using a method whereby B) a sample is introduced into the first station where it is filtered, the resulting plasma wicks to the channel on the middle strip layer where lysis/adsorption of the target material occurs, and the plasma then wicks through the wash channel in the third member, C) the strip is slid to the second station where a drop of wash buffer is introduced and then wicks to the wash channel on the third member, D) the strip is then slid to a region where the wet channel can dissolve dried amplification reagents, E) the strip is further slid to a region where the channel region is hermetically sealed and heating/amplification can occur, and F) the strip is then further slid to a region where the solution in the strip channel is allowed to contact a colorimetric reagent stored in the fourth station of the device;

FIG. 14 shows a scheme where layers of PET (with a hole cut out to house a 4.8 mm Whatman 4 paper disc) were laminated together to create a chamber that prevents evaporation during the incubation period, while still allowing post-reaction access to the disc for analysis;

FIG. 15 shows the fluorescent signal and standard error of the mean signal obtained following detection of LAMP reactions conducted in Whatman 4 paper discs carried out with varying amounts of starting genomic material (n=7), where the horizontal line labeled LOD represents the limit of detection calculated as 3 times the standard deviation of the no amplification negative control;

FIG. 16 shows the effect of reactor materials on LAMP reactions, where LAMP reactions were carried out on Whatman 4 paper discs containing 10,000 starting genomic copies of DNA in reactor devices constructed using various adhesives and greases;

FIG. 17 shows an exploded view of the sliding strip device used to demonstrate strip-based LAMP amplification using optimal materials;

FIG. 18 shows the fluorescent signal detected when strip-based LAMP amplification is carried out using varying amounts of starting genomic material in a device that incorporates optimal materials;

FIG. 19 illustrates sample preparation steps on a sliding strip device using fingerstick whole blood and a drop of PBS buffer by the following steps: (A) sample is introduced to the device, (B) blood sample wicks through the disc to the wash channel, (C) a drop of PBS buffer is applied, (D) the strip is slid so that the disc is in contact with the buffer, (E) the buffer is wicked through a second wash channel, and (F, G) the strip is removed;

FIG. 20 shows an exploded perspective view of a device comprising a plurality of parallel-disposed sheets (panel a), schematic diagram illustrating a method for performing an assay using the device (panel b), and read guides for quantifying the results of the assay (panel c);

FIG. 21 shows a liver enzyme test device that includes two tests and three controls and exemplary result outputs;

FIG. 22 illustrates designs for multiplexed devices;

FIG. 23 is a diagram useful in illustrating a method of manufacturing a plurality of devices;

FIG. 24 illustrates a plasma separation membrane filter attachment process in a device fabrication method;

FIG. 25 shows an exploded view of a device configured for quantitative colorimetric readout (left panel) and exemplary assay readouts (right panel);

FIGS. 26A and 26B are bottom and top views of a liver enzyme test device;

FIG. 27 depicts a four-layer analytical device;

FIG. 28 depicts an analytical device;

FIG. 29 depicts a six-layer analytical device;

FIG. 30 depicts a five-layer analytical device;

FIG. 31 depicts a seven-layer analytical device made from patterned paper containing a single sample input;

FIG. 32 depicts a seven-layer analytical device containing a multi-wash ELISA design;

FIG. 33 shows a calibration plot of the output signal versus the concentration of hCG in buffer sample, for an analytical device described herein; and

FIG. 34 depicts a six-layer analytical device.

### DETAILED DESCRIPTION

The invention provides molecular diagnostic test devices and methods for using such diagnostic test devices to detect analytes of biological significance in a patient. The diagnostic test devices are particularly useful for detecting a polynucleotide analyte in a sample obtained from a patient. Further, the diagnostic test devices are inexpensive, disposable, easy to use, and are useful at the point of care. Various aspects of the invention are set forth below in sections; however, aspects of the invention described in one particular section are not to be limited to any particular section. For example, exemplary analytes for detection (i.e., targets), exemplary detection limits for the analytical devices, exemplary structures of the devices, exemplary materials used to prepare the devices, and exemplary methods of manufacturing the devices are described below.

### Targets

Favorable targets will be those that are clinically important, easily isolated, robust, and amenable to amplification, preferably by isothermal methods (including existing probes, primers, etc). It is appreciated that RNA targets and DNA targets can be detected using devices described herein. Particular analyte candidates include: E. coli, S. aureus, hepatitis B virus (HBV), hepatitis C virus (HCV), cytomegalovirus (CMV), human immunodeficiency virus (HIV), tuberculosis (TB), and malaria. Endogenous human or animal DNA or RNA can also be isolated and amplified using devices described herein. Depending on the amplification assay employed, one can measure the presence or absence of genomic DNA, allelic gene variants, gene mutations, small nucleotide polymorphisms, and different species, transcript variants, and post-transcriptional processed forms of RNA.

### Detection Limit

In certain embodiments, the target detection limit is 1,000 copies/mL. This target detection limit takes several factors into consideration: i) a 30 μL finger stick sample at a concentration of 1,000 copies/mL will contain just 30 copies of oligonucleotide which approaches the amplification limit for several isothermal methods (see Table 1); and ii) 1000 copies/mL is a clinically relevant concentration for several disease targets including: HBV¹, CMV², HCV³, and malaria⁴.

### Structure of the Device

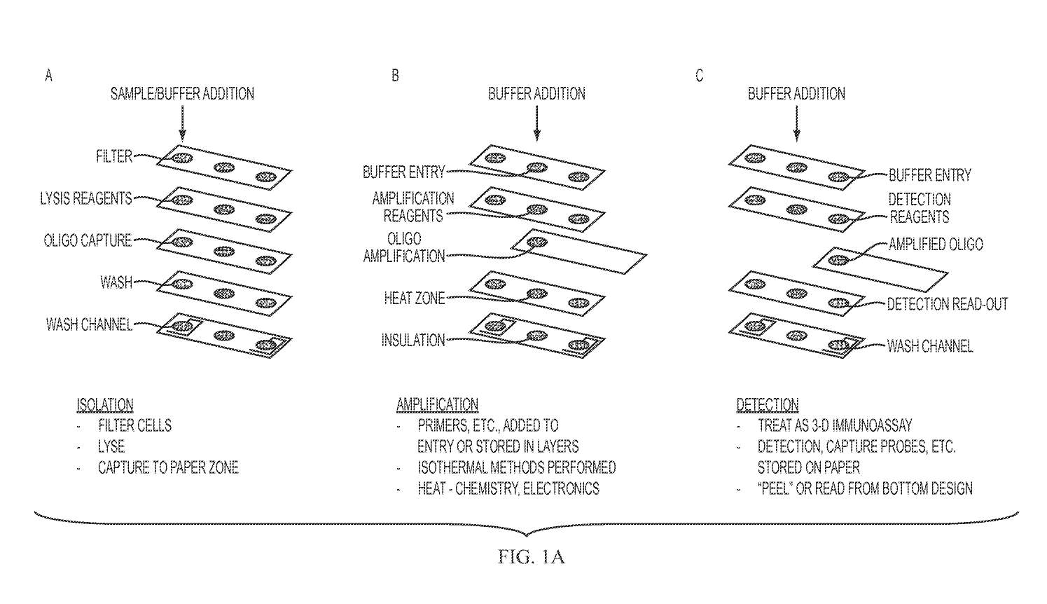

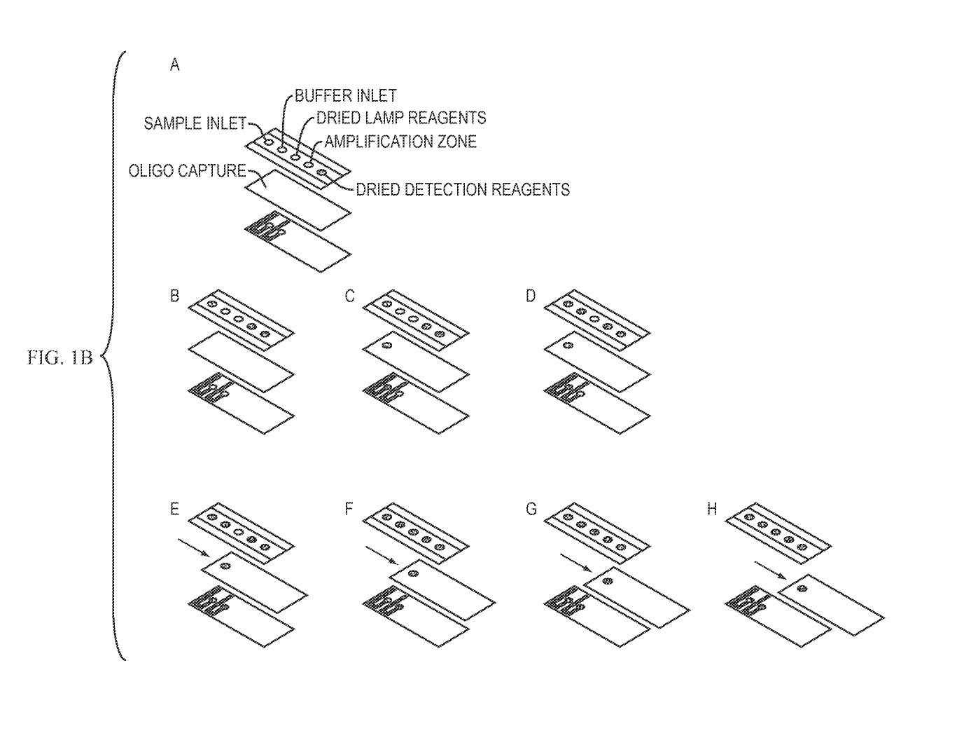

The nucleic acid detection device carries out three principal processing steps: i) isolation and purification of target nucleic acids, ii) amplification of target nucleic acids, and iii) detection of the amplified species with an optical or visual readout. A single paper-based device is capable of conducting each of these steps. While reference herein is to “paper,” and various adsorptive papers are one type of preferred media for fabrication of the devices, “paper” as used herein, is intended to include any adsorptive, porous sheet or sheet-like material that can transport liquids through wicking, wetting, adsorptive or absorptive fluid flow, e.g., nitrocellulose sheets such as are commonly employed in the familiar lateral flow pregnancy test kits. To accomplish this, we exploit a two dimensional or three dimensional “sliding strip” format of microfluidic patterned paper platform. A 3-dimensional device exploiting liquid flow between parallel disposed sheets (FIG. 1A) is preferred, but two dimensional devices where liquid flows laterally in an adsorptive sheet and across edge boundaries between the sliding member and the stationary members also may be used. The format allows for one or more layers to slide to different positions along the device making fluidic contact with other channels/zones not previously connected in the original position. With such a format, it is possible for a single fluidic zone containing analytes (captured oligonucleotides, for example) to be exposed serially to various chemistries and processing steps, each independent of the other.

FIG. 1A is an exploded, perspective schematic illustrating an exemplary embodiment of the structure and operation of a 3D sliding strip device which serially processes a sample in three steps for the detection of nucleic acids. A) The sliding strip defines a test zone, labeled as “oligo capture”, and is in the middle of a stack of five strips positioned at a first station to receive a filtered blood sample and capture RNA or DNA after lysis. A wash channel is located beneath to remove lysis products, etc. B) The strip containing the test zone, an oligonucleotide capture spot, is slid laterally to register the test zone with other features of the device at the second station where the now captured and purified oligonucleotides are placed in an amplification position. Here, upon addition of buffer to an inlet, they receive reagents necessary to perform amplification, preferably an isothermal form of amplification. Optionally, a heating zone, driven by exothermic reactions or simple electrical resistors, may be located underneath the reaction zone. C) The now amplified oligonucleotides are moved by additional lateral sliding of the sliding strip to a third station where they are placed in position to be detected. This may be effected using, for example, colloidal gold conjugates, DNA intercalating agents, detection probes, and/or capture antibodies or oligonucleotides dispose in the device in dried form and brought into contact by addition of buffer at the third station. A wash channel may be provided to wash away unbound reagents. A portion of the device may be peeled away to reveal a colorimetric result indicating the presence or absence of the target sequence. A more detailed consideration of each processing step is described below.

FIG. 1B is an exploded, perspective schematic illustrating an exemplary embodiment of the structure and operation of a 3D sliding strip device which serially processes a sample in seven steps for the detection of nucleic acids. A) The sliding strip defines a test zone, labeled as “oligo capture” located in the middle of a stack of three or more strips and initially positioned at a first station to receive a filtered blood sample and capture RNA or DNA after lysis. B) A drop of blood is applied to the first station where it is filtered, and C) wicks vertically through the paper disc located on the strip and into a wash channel located beneath. At this stage, the target organism is lysed and its nucleic acid material is adsorbed onto the paper disc. In this manner, the sample is concentrated as nonadsorbed components wick through to the wash channel. D) A drop of wash buffer is placed into the second station and E) the strip containing the test zone is slid laterally to register the oligonucleotide capture spot with the second station. The drop of buffer is wicked vertically through the test zone and into a second wash channel located beneath. F) The strip, now containing purified nucleic acid material, is slid to a third station whereby it contacts and absorbs dried reagents necessary to perform amplification, preferably an isothermal form of amplification. G) The strip is slid again to a hermetically sealed reaction zone where the disc is heated, by exothermic reactions or activation of simple electrical resistors, which may be located underneath the reaction zone. The heating drives the amplification reaction at a pre-determined temperature. H) Finally, the now amplified oligonucleotides are moved by additional lateral sliding of the sliding strip to a fifth station where they are in position to be detected. This may be effected using, for example, colloidal gold conjugates, DNA intercalating agents, detection probes, and/or capture antibodies or oligonucleotides disposed in the device in dried form and absorbed by being placed in contact with the wet disc or by the addition of buffer. A wash channel may be provided to wash away unbound reagents. A portion of the device may be peeled away to reveal a colorimetric result indicating the presence or absence of the target sequence. A more detailed consideration of each processing step is described below.

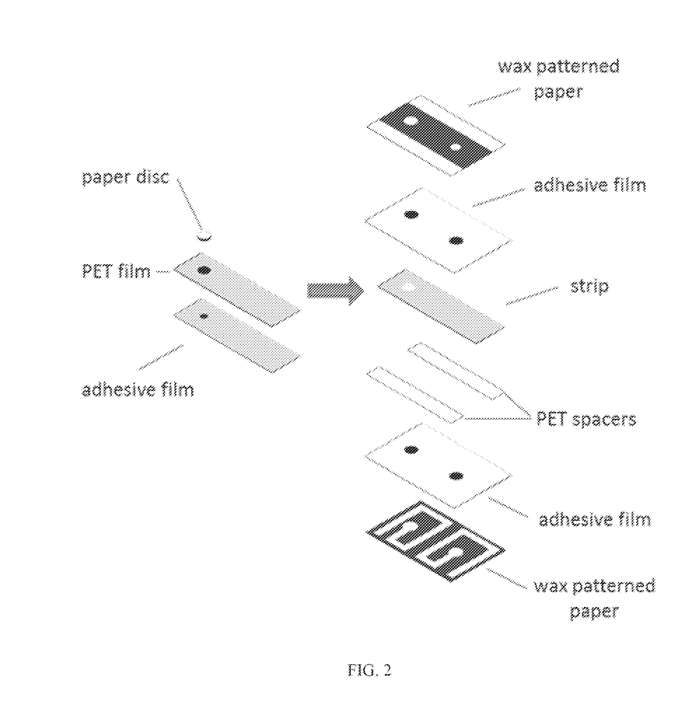

In some embodiments, the device may comprise a stack of five planar members capable of processing a sample for detection of nucleic acids (FIG. 2). The top planar member may comprise a strip of paper patterned with a hydrophobic substance to form two hydrophilic regions suitable for introduction of a liquid sample or reagent. The middle strip defining a test zone is situated between two strips of adhesive film with engineered apertures aligned with the inlets of the top strip of paper. The bottom strip of paper may be situated below the lower strip of adhesive film and may contain a hydrophobic substance patterned to create two channels, the commencement points of which are aligned with the apertures and inlets of the strips above and can draw liquid from the above layers.

In a first step, a sample is introduced into the top entry point on the device where it flows to the paper disc in the sliding strip layer. A filter membrane may be incorporated to filter sample components. Lysis chemistry present in the paper disc may then lyse unwanted components of the sample while chemical treatments present on the paper disc simultaneously adsorb nucleic acids. The disc may then be slid to the second region of the device where it encounters a drop of buffer which passes through to the second wash channel carrying unabsorbed components away from the test zone. The strip containing the test zone may then be slid laterally to absorb dried nucleotide amplification reagents. Alternatively, nucleotide amplification reagents may be added directly to the second inlet to facilitate amplification of captured nucleic acids. In some embodiments, the device may comprise a third hydrophilic region patterned on the uppermost strip, aligned with the other two described hydrophilic regions and positioned such that when the sliding member is pulled further in the same direction along its previous trajectory past the second inlet region, the region of the sliding strip containing the paper disc encounters the third inlet. Nucleotide amplification reagents may be added directly to the third inlet to facilitate amplification of captured nucleic acids.

In the above device, nucleic acid amplification may be achieved by heating the entire device isothermally. A heating zone, driven by exothermic reactions or simple electrical resistors, may be located underneath the zone containing or to which are added polynucleotide amplification reagents to facilitate nucleic acid amplification. In a preferred embodiment, a printed inert grease which allows for a hermetic seal during heating is incorporated into the device. Following nucleic acid amplification, the sliding member may optionally be removed from the device and dried. A detection reagent may then be added to the test zone containing the amplified oligonucleotides and detection achieved via a suitable assay.

### Materials

A primary concern in the design of the device is the choice of materials. For the sliding strip to function properly, the disc which comprises the site of the various reaction steps must remain sealed from the external environment while providing fluidic contact between the disc and the various reagents added at each inlet. The disc must also provide exclusive contact with individual regions of the device comprising the different stations as it is moved through the device such that the disc does not maintain fluidic contact with any other station when positioned at a particular station. Creation of an evaporation-resistant seal can be achieved by incorporation of grease into the device, such that a layer of grease is placed on the top surface of the sliding strip member exclusive of the area comprising the reaction disc. In a preferred embodiment, Krytox® fluorinated polymer grease is used to create such a seal.

For ease of fabrication, it is preferable to use an adhesive substrate as the base material for the sliding strip planar member of the device. In a preferred embodiment, a low-tack PET film forms the base of the device.

Nearly any porous material can be patterned by methods disclosed herein. Materials include, but are not limited to: paper, chromatography paper, nitrocellulose, non-woven polymeric materials, lab wipes, nylon membranes such as Immunodyne® membranes sold by Pall® corporation. A preferred material for the present invention is Whatman® no 1. chromatography paper.

In some embodiments, stabilizers may be added to the reagent zones to further stabilize the enzymes spotted onto the paper. In further embodiments the stabilizers include but are not limited to: trehalose, poly(ethylene glycol), poly(vinyl alcohol), poly(vinyl pyrrolidone), gelatin, dextran, mannose, sucrose, glucose, albumin, poly(ethylene imine), silk, and arabinogalactan. In some embodiments, dye stabilizers such as MgCl₂ or ZnCl₂ may be added to the assays.

In preferred embodiments, the stabilizers are sugars. A particularly useful method for stabilizing enzymes and other proteins, vacuum foam drying, is described by Bronshtein et al. in U.S. Pat. No. 6,509,146, which is incorporated herein by reference in its entirety.

### Methods of Manufacture

Multiple layers of a test device may be held together by an adhesive. Any suitable adhesive may be used. For example, in some instances, a hydrophobic, polymeric, adhesive may be used. In further embodiments, the adhesive may be patterned by a printing technique including, but not limited to, screen printing, flexographic printing, gravure printing, transfer printing, and ink jetprinting. A preferred embodiment is to pattern the adhesive by screen printing. Whitesides et al. report a method for adhering multiple layers of patterned paper together using double-sided tape cut with a laser cutter (Proc Natl Acad Sci 105:19606-19611, which is incorporated herein by reference in its entirety). When the cut double-sided tape is used, it leaves a gap caused by the thickness of the tape and prevents contact between the hydrophilic regions of the patterned paper. This gap must be filled with cellulose powder to enable z-direction flow (i.e., tangential flow through the device). Screen printing of adhesives offers several advantages over this technique. For example, the patterned adhesive layer typically can be applied in very small thicknesses (e.g., between about 1 and about 500 microns, between about 1 and about 100 microns, between about 1 and about 50 microns, and between about 50 and 100 microns), which allows for intimate contact to occur between the hydrophilic regions of the patterned paper and eliminates the need to use the cellulose powder filler. Screen printing may also require much less material than double-sided tape, which reduces device raw material cost. Furthermore, screen-printing is a low-cost and easily scaled patterning technique, which is advantageous for inexpensive, mass production of the test devices. In a preferred embodiment, the adhesive may be a pressure sensitive adhesive.

The manufacturing unit operations for a test device can be separated into a series of steps. For example, in some embodiments, the manufacturing operations may include some or all of the following steps: patterning of the paper substrate with hydrophobic barriers, patterning of adhesive by screen printing, deposition of biological/chemical reagents, layer alignment and assembly, attachment of plasma separation membrane, and/or lamination and packaging.

A preferred method for patterning paper to be used in a test device is wax printing, although any suitable method for creating hydrophobic barriers on a porous, hydrophilic sheet may be used. Wax printing is described in detail by Whitesides et al. in Anal Chem 81:7091-7095 and International Patent Application Publication No. WO 2010/102294, both of which are hereby incorporated by reference in their entirety. The device may be designed on a computer and the hydrophobic walls of the microfluidic channels may be printed onto a sheet of paper using a commercial printer with solid-ink technology (e.g., using a Xerox Phaser printer). The printer generally operates by melting the wax-based solid ink and depositing the ink on top of the paper. The sheet is then heated to above the melting point of the wax, allowing wax to permeate through the thickness of the paper, thereby creating a hydrophobic barrier through the entire thickness of the paper. In some cases, spreading of the wax may occur during the heating step, but the spreading is reproducible based on the type of paper used and the thickness of the printed line and can be incorporated into the design. Without wishing to be bound by any theory, it is believed that the channels patterned in the paper wick microliter volumes of fluids by capillary action and distribute the fluids into test zones where independent assays can take place.

Other method embodiments may use paper soaked in photoresist which is then exposed to UV light through a photomask with a desired pattern. The unexposed regions are then washed away with a suitable solvent, leaving behind crosslinked hydrophobic regions that penetrate the thickness of the paper. Feature sizes as small as 100 μm have been demonstrated using this technique. Examples of this method of patterning can be found in prior work from in Angew. Chem. Int. Ed. 2007, 46, 1318-1320 and International Patent Application Publication No. WO 2008/049083, which is hereby incorporated by reference in entirety. In further embodiments, there is a host of other large-scale printing and patterning techniques that can be used to deposit hydrophobic barriers into paper to meet the requirements of the test device. These methods include, but are not limited to: screen-printing, gravure printing, contact printing, flexographic printing, hot embossing, ink jet printing, and batik printing.

In several embodiments, the layers may be adhered together in such a way that fluids can wick in the z-direction (i.e., tangentially) to entry points in the next layer of paper. One method of accomplishing this is by using double-sided adhesive tape with holes cut into the desired pattern through which fluid can flow. This method is described in more detail in Proc. Natl. Acad. Sci. USA, 2008, 105, 19606, which is hereby incorporated by reference in entirety. In this particular method, a hydrophilic powder (i.e., cellulose) may be added in the cut aperture between the layers of paper formed by the thickness of the tape. A preferred method for assembly of 3-D devices is to use simple and scalable screen-printing techniques to deposit very thin layers of adhesive onto paper in the desired pattern. In this manner, a hydrophobic, pressure-sensitive adhesive (e.g., Unitak 131 sold by Henkel Corporation) can be applied to the paper. Once adhesive is applied, pre-made sheets can be stored by laminating the adhesive side to a non-adhesive release layer, for example as commonly seen in other adhesive products such as labels and tapes. In further embodiments, a stencil can be fabricated and pressed against a sheet of patterned paper in such a way that certain features are covered. An adhesive may then be deposited from an aerosol spray onto the remaining exposed regions.

In preferred embodiments, it is necessary to deposit chemical and/or biological assay reagents into regions of the device. The reagents react with analytes present in a bodily fluid or which are amplified from analytes present in a bodily fluid (e.g., polynucleotide analytes) and which yields a response (i.e., colorimetric or electrochemical) that can indicate the concentration of a particular analyte. In some embodiments, it is often necessary to formulate reagents with appropriate stabilizers (e.g., sugars) to preserve function once dried. In one embodiment, useful for prototyping and small scale production (e.g., 100's of devices per day), deposition of reagents is done by hand using micropipettes and repeat pipetters. A typical volume deposited is between 0.5 and 5 μL. In preferred embodiments for larger scale production, precision liquid deposition machines can be used. Two examples of such tools are the AD3400 available from BioDot, Inc. and the Diamatix DMP-2800 Ink Jet printer available from Fujifilm. Both of these units are able to rapidly dispense precise volumes (contact-free) of fluid down to nL volumes in a programmed pattern. Additionally, such units can be adapted to continuous manufacturing lines for large scale production.

In preferred methods of manufacture, devices are assembled in full sheets. For this to occur, it is imperative that patterned regions precisely align to make the necessary fluidic junctions possible between layers. A simple and scalable way to accomplish this is to cut precise holes in the paper layers such that the sheets can slide onto peg boards. Each layer can then be applied to the peg board such that features are rapidly aligned correctly. In continuous manufacturing, a similar method can be used on reels containing pegs such as that used in Dot-Matrix Printing. Alternatively, laser web guides can be used to precisely align sheets before lamination. Other methods for aligning the sheets will be known to those of ordinary skill in the art.

A plasma separation membrane (Pall Corporation) may be placed at the first station of the device. The membrane may serve as a reservoir to collect a biological fluid (e.g., a blood drop) and importantly to filter cells (e.g., red blood cells) out of the biological fluid and allow fluid (e.g., plasma) to wick into the device zones. Accordingly, certain embodiments of the present invention utilize a “pick and place” method consisting of the following steps:
(i) A sheet of Pall membrane may be cut into densely packed circles 1 cm in diameter using a die cutter. The die used for cutting is designed such that the filters remain in place after cutting.
(ii) A sheet of adhesive laminate may be cut using a knife plotter, laser cutter, die cutter, or the like such that it contains apertures which act as an entry point into the filter/device. The holes in the laminate sheet may be between about 0.1 cm and about 1.5 cm in diameter or between about 0.5 cm and 1.0 cm. In a preferred embodiment, the holes are about 0.75 cm in diameter.
(iii) A non-adhesive masking layer may be cut, e.g., from waxy cardstock, or other materials with low adhesion, in a pattern to have holes that are larger than the filters. For example, in some embodiments, the diameter of the holes in the non-adhesive masking layer may be more than about 0.2 cm, more than about 0.3 cm, more than about 0.4 cm, or more than about 0.5 cm larger than the diameter of the holes in the membrane. In a preferred embodiment, the holes in the masking layer are about 1.13 cm in diameter.
(iv) The previously cut laminate containing 0.75 cm holes and the masking layer may be adhered together such that the laminate aperture is in the middle of the blocking layer aperture.
(v) The stack may be placed over the discs (in registration on the die plate) in such a way as to only pick up filter membrane discs that align with the cut laminate sheet.
(vi) The adhesive laminate layer is peeled away which, as it is peeled, adheres a filter over each laminate aperture on the laminate.
(vii) The laminate layer, now with a filter membrane adhered under each aperture, may be adhered to a stack of two layers of patterned paper which may be adhered together by screen printed adhesive. In this way, the maximum area of the membrane material can be converted into useable filtration discs for devices. Using die-cutting techniques and simple laminators, this process can be easily automated into large scale-production.

After the steps above have taken place, the stack of patterned paper (and filters, etc., if required) may be laminated. In some embodiments, a “cold lamination” sheet consisting of a PET film with adhesive on one side may be used. The film protects the devices and provides the outer hydrophobic layer for the patterned zones. The device elements may then be separated into separate devices (e.g., cut into separate devices). In some embodiments, the devices may be placed in foil-lined bags and heat sealed, preferably where the bags contain a desiccant.

In some embodiments, it is useful to have certain sample handling features built into the device itself. For example, one such feature is a simple plastic cover that protects the sample entry aperture. After a drop of biological fluid is introduced to the device via the entry aperture and into the filter membrane, a plastic cover may then seal the aperture to slow the evaporation and drying of the fluids in the device.

### Isolation

In order for target oligonucleotides to be amplified and ultimately detected, they must first be isolated, captured, and purified. FIG. 1A, part A and FIG. 1B, part A illustrate potential designs for isolation of viral or bacterial nucleic acids from a blood sample. The process may be broken down into several steps, each of which take place in a separate layer/zone of the respective device.

Sample Procurement—In some embodiments, it may be desired to have a built-in capillary capable of drawing a precise volume of blood into the device by simply making contact with the droplet. Such a feature can minimize user operations and ensures reproducibility in the volume of sample introduced to the device. In still further notional embodiments, a test device may contain a built-in lancet, which is disposed of along with the device after use.

In some embodiments, the device may be used as part of a kit containing a glass or plastic capillary tube. In preferred embodiments the tube is plastic, such as the MicroSafte Tube available from Safe-Tec®. In some embodiments, the kit may contain a lancet. In preferred embodiments, the lancet is a spring-loaded lancet, such as those available from Surgilance™. In still further embodiments, a kit will contain patterned paper devices, a lancet, a capillary tube, a bandage, an alcohol swab, latex gloves, and a colorimetric read guide for interpretation of results.

Filtration of Red and White Cells to Isolate Plasma—

There is considerable experience and literature concerning plasma separation membranes, which effectively isolate plasma and allow it to wick into detection zones that contain chemistry to detect solutes disposed therein. Membranes such as Vivid GX plasma separation membrane available from Pall® corporation are highly effective. In other embodiments, the membrane can be a glass fiber membrane, or even a paper filter. In other embodiments, anti-blood cell antibodies may be attached to the membrane to facilitate capture of cells. In further embodiments, “scrubbing agents” may be added to the filter membrane or paper channels that are capable of capturing substances that may interfere with the reaction chemistry.

Lysis of Virus/Bacteria—

A number of chaotropic reagents exist that can be used to lyse cells, viruses, and bacteria. A common chaotropic reagent is urea, which could be dried onto a paper zone and act as a lysing agent once in contact with a sample. Alternatively, paper products such as FTA® cards available from Whatman® contain proprietary agents embedded within the paper to lyse membranes and denature viral coat proteins.^{5,6}

Other lysing strategies also may be exploited. For example, cells may be lysed by a suitable reagent in one zone followed by digestion of DNA or RNA in the same or a downstream layer, followed by filtration of particulates in another downstream layer, optionally followed by addition of buffer as a wash, and/or provision of a downstream liquid reservoir that draws fluid vertically through the stack. Additionally, a heating element may be incorporated into the device to thermally lyse cell membranes or denature viral coat proteins. Nucleotide species may be captured on the membrane directly following cell membrane or viral coat lysis, or restriction endonucleases or other appropriate nucleotide lysing reagents such as particular restriction enzymes may be incorporated or added to the device.

Adsorption of Oligonucleotides from Plasma onto Membrane—

A variety of membranes and surface treatments can effectively adsorb or “capture” target oligonucleotides. A particularly attractive material which claims to accomplish both lysis and capture is the paper FTA® cards available from Whatman⁵. These products are easily patterned using the technologies disclosed, for example, in PCT applications WO/2008/049083, WO2009/121037, PCT/US2010/026547, incorporated herein by reference, and in the technology disclosed herein. These products also are frequently used to lyse and immobilize oligonucleotides from samples. The membranes hold onto the target oligonucleotides while washing occurs and PCR, or other isothermal amplification techniques, can be performed directly from these captured molecules.^{5,6} Incorporation of buffer washes using reagents like phosphate buffered saline (PBS) can wash away debris and non-adsorbed oligonucleotides in preparation for amplification steps. Washing may be improved by incorporation of wash channels into the device that provide a means for drawing away non-adsorbed materials.

### Amplification

The past decade has seen tremendous advances in the field of isothermal amplification techniques for RNA and DNA.⁸ Each method has its own advantages and disadvantages which are often specific to the platform being used or the target one wishes to detect. Such amplification techniques are contemplated for use in the paper-based devices described herein at the amplification station. Factors such as target sequence, speed, stability of reagents, and interference from paper (or other porous media) are considered to optimize these methods.

The Table below lists a variety of isothermal amplification techniques available for use at the amplification site. A particularly attractive technique is Loop-mediated isothermal amplification (LAMP).^{6,8,9} LAMP has the following characteristics which make it attractive for use on the microfluidic paper platform: (i) it works well with DNA (or RNA when used in combination with reverse transcriptase), (ii) it is highly specific to the target sequence because it uses a total of four primers, (iii) it can be performed in less than one hour, (iv) it has been demonstrated using DNA adsorbed to paper substrates⁶, (v) its reagents can be stored in lyophilized form,⁹ and (vi) the reaction solution becomes turbid as a result of pyrophosphate formation during amplification so LAMP potentially has a built in read-out mechanism, simplifying detection. Additionally, by-products of LAMP amplification can be fluorescent, providing another built-in detection mechanism. Although LAMP has several attributes which make it beneficial for this invention, other methods have advantages with respect to reaction time, reaction temperature, and number of reagents necessary. Thus, the particular method used should be the result of screening, and the selected method being optimized for efficacy and reproducibility.

| | Detection | Reaction | Reaction | |
|---|---|---|---|---|
| Method | Limits | Temp (° C.) | Time | Target |
| Loop mediated | 6 copies | 65° | 60 min | DNA or |
| isothermal | | | | RNA |
| amplification | | | | |
| (LAMP) | | | | |
| Helicase | 10 copies | 64° | 90 min | DNA or |
| Dependent | | | | RNA |
| Amplification | | | | |
| (HDA) | | | | |
| Recombinase | 10 copies | 37° | 20 min | DNA |
| Polymerase | | | | |
| Amplification | | | | |
| (RPA) | | | | |
| Nucleic Acid | Comparable | 65°-5 min | 105 min | RNA |
| Sequence Based | to PCR | 41°-100 min | | |
| Amplification | | | | |
| (NASBA) | | | | |
| Transcription | <50 copies | 95°-10 min | 140 min | RNA |
| Mediated | | 42°-65 min | | |
| Amplification | | 60°-25 min | | |
| (TMA) | | | | |
| Rolling Circle | 60 copies | 37° | ~60 min | DNA |
| Amplification | | | | |
| (RCA) | | | | |
| Strand | 46 copies | 37° | 120 min | DNA |
| Displacement | | | | |
| Amplification | | | | |
| (SDA) | | | | |

In one embodiment, the device can be heated at 65° C. for 1 hour to facilitate oligonucleotide amplification. Optionally, this can be achieved by placing the entire device in an oven or other suitable device set to the appropriate temperature. Following this isothermal amplification step, the sliding member comprising the test zone can be removed from the device and dried at 65° C. for 5 minutes before addition of a detection reagent.

Thermal cycling amplification techniques also may be used, but are less preferred because they require an apparatus for reproducibly changing temperature at the oligo amplification site which may not be available and/or may be hard to provide at the point of use.

An important consideration for applying any isothermal technique to a paper device is that all of these methods require heat to drive the amplification reaction. This step can be done simply by placing the device in an oven set at an appropriate temperature. However there are several ways to incorporate a heating element into a device. One method is to pattern an electric resistor into the portion of the device where heating is required. The Whitesides lab has used this technique to create valves and concentrators on paper microfluidic devices.¹⁰ See, for example, WO 2009/121041. The resistor can be operated using a “button battery” at a cost less than $0.10.

Another integrated heating option is the use of exothermic chemical reactions such as those found in “meals ready to eat” and commercial hand warming products. Weigl, et al.⁹ have shown that by coupling these types of reactions to carefully chosen phase-change materials (such as waxes or metal alloys) it is possible to maintain constant elevated temperatures, well within the range needed for isothermal amplification, up to several hours. These techniques may be adapted to paper microfluidic devices to provide region-specific, sustained heating to isothermal amplification chemistries.

An important consideration during the amplification heating step is evaporation. This can be mitigated through lamination of the device and the use of reversible, sealable flaps over entry ports, etc. Another approach takes advantage of the enclosed form factor inherent to the sliding architecture described above. For example, the reaction zone may be slid to a region possessing top and bottom barriers, forming a temporary hermetic seal to prevent evaporation during heating.

### Detection

Detection Reagents and Methods—

After amplification has occurred, the strip is moved to a station to facilitate detection (FIG. 1A, panel C; FIG. 1B, panel H). Given the precedence for performing amplification reactions directly from adsorbed DNA or RNA on surfaces, this can be done using known techniques. There are several strategies that can be used for detection resulting in a colorimetric readout on the device.

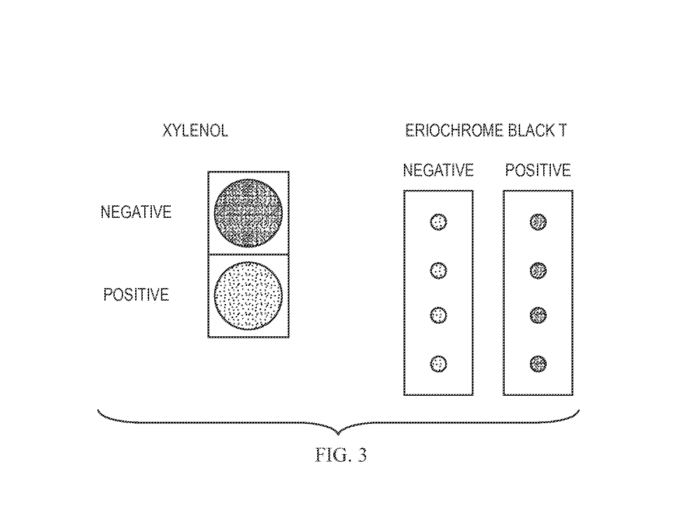

One attractive detection strategy when LAMP is used to perform nucleotide amplification is the incorporation of magnesium sensitive dyes. Amplification via LAMP produces an insoluble precipitant, magnesium pyrophosphate. Thus, in a reaction during which little or no amplification occurs, magnesium is available for conjugation to such a dye, whereas in a reaction during which a high degree of amplification occurs such that the majority of magnesium is incorporated into magnesium pyrophosphate, free magnesium is not available for dye conjugation. FIG. 3 shows two such dyes that are particularly useful in this context. Xylenol orange turns from red to yellow in the presence of magnesium (FIG. 3A), and eriochrome black T turns from blue to red in the presence of magnesium (FIG. 3B). Other dyes known in the art for the detection of magnesium are applicable.

A recently developed technology that may be useful in the detection process is the synthesis of capture oligonucleotides using paper as the substrate for synthesis, with one end of the synthesized molecule tethered to functionalized paper fibers. A potential advantage of this approach is the extremely high densities (5×10¹⁴ molecules/cm²) achieved owing to the greater surface area of paper when compared to a solid, planar surface such as glass.

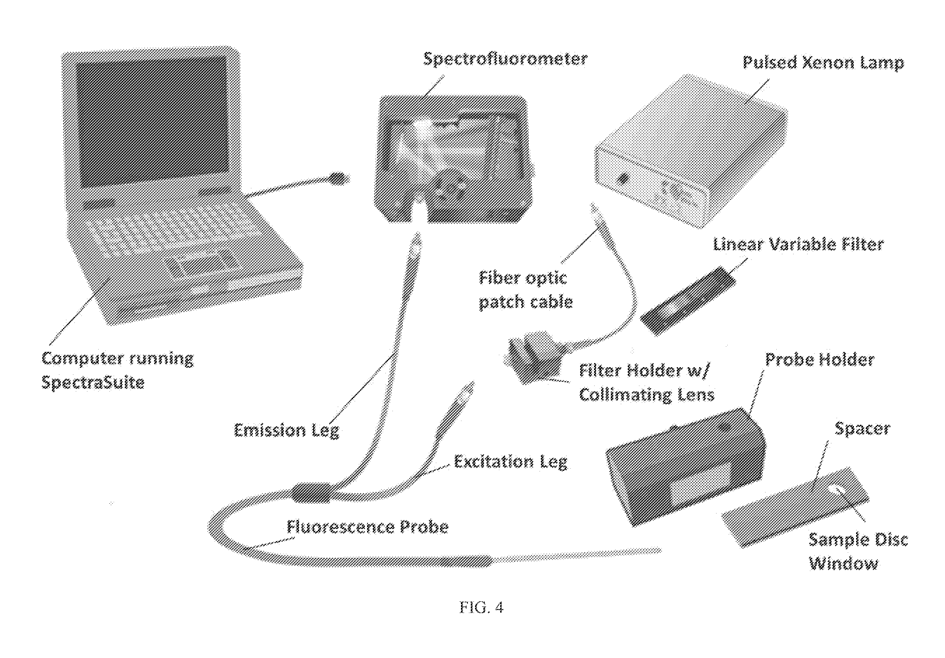

Oligonucleotide amplification can also be detected via application of reagents that fluoresce at specific wavelengths following intercalation between nucleic acid base pairs. Such reagents include Propidium iodide (PI), SYTO® Green (Invitrogen), and SYBR®-Green (Applied Biosystems). In order to quantify oligonucleotide amplification, a detection system comprising a pulsed xenon lamp light source, a linear variable filter holder equipped with a linear variable filter, a probe holder capable of holding the sample and detection probe, a spectrofluorometer, a computer capable of processing spectrofluorometric data, and a cable comprising an excitation, emission, and probe leg connecting the various components can be used (FIG. 4). The excitation leg of the cable is connected to the xenon lamp via a linear variable filter holder and patch cable, allowing shaping of the excitation spectrum. The emission collection fiber leg is connected to the spectrofluorometer for recording the final PI emission spectra. Following application of PI to the test zone containing the amplified oligonucleotide products, the planar member containing the test zone can be placed in the probe holder. The xenon lamp can provide light of a wavelength excitatory to intercalated PI that passes through the variable filter and the excitation leg of the filter, sequentially, to the fluorescence probe. Fluorescent signal emitted from the sample is detected by the probe and communicated via the emission leg of the cable to the spectrofluorometer for readout. The computer is used to analyze the captured emission data. In this manner, the detection system can be used to measure the amount of intercalated PI and thereby measure overall amplified oligonucleotide content in the test zone. In this embodiment, a separate detection zone incorporated within the device is unnecessary.

Electrochemical detection of amplified nucleic acids, such as that described by Lu, et al. in Anal. Chem., 2012, 84 (4), pp 1975-1980, is also possible.

Several groups have reported the detection of amplified oligonucleotides using a lateral flow platform, referred to as nucleic acid lateral flow (NALF).^{11,12,13,14} These assays are akin to lateral flow immunoassays where detection reagents are conjugated to colored particles (typically colloidal gold) such that they can bind to a target sequence on an amplified product. This initial complex is then captured onto a membrane using a “capture oligonucleotide.” Various versions of this concept are known and are amenable to the paper microfluidic platform. For example, Lemeiux, et al. used a FITC-labeled “capture probe” which is coupled with an anti-FITC antibody immobilized on a membrane.¹¹ In some embodiments, a detection reagent such as an antibody is conjugated to another entity such as an enzyme. In preferred embodiments, the enzyme is horseradish peroxidase or alkaline phosphatase. In further embodiments, the antibody conjugate is in the form of one or more antibodies linked to a colored particle. In some embodiments, the particle is selected from, but not limited to, the following: a colored polymer latex particle, a colloidal gold particle, a graphite particle, a quantum dot, or a carbon nanotube. Thus, in some embodiments, an antibody or multiple antibodies can be used to detect and capture an amplicon labeled with an optically detectable probe. In some embodiments, an antibody selected to detect a moiety comprising part of a probe with affinity for a specific polynucleotide amplicon may itself be labeled with an optically detectable particle such that binding of the probe by the labeled antibody provides a means of detecting polynucleotide amplicons.

We have successfully demonstrated and reported several immunoassays (hCG, C. difficile, Bt Cry1Ab) on patterned paper in a multilayer, 3-Dimensional format capable of sample processing, reagent storage and release, programmed incubation time, capture, and washing. Furthermore, sensitivity and repeatability were comparable to commercial rapid tests. Multiplexing of several tests on one device has also been demonstrated using this design. This previously developed architecture and techniques, disclosed in the literature and throughout this disclosure, will be useful in the detection of amplified target oligonucleotides.

Antibody-Mediated Detection Methods—

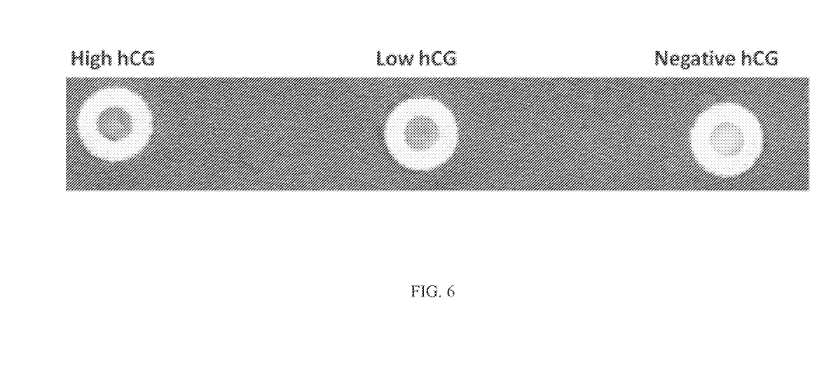

FIG. 6 illustrates a method for capturing and detecting the presence of an analyte from a sample inserted into a paper-based fluid flow device using antibodies. In this example, a single drop of antigen-containing sample is placed into the top of the device, where it encounters the reagent storage layer, where antibody conjugates in the form of antibody-coated colored particles (such as latex or colloidal gold) have been previously spotted and formulated to release into solution once in contact with the liquid sample. The fluid sample then encounters the dwell layer, which is slightly hydrophobic but still permits wicking and acts to provide a programmed incubation time for a partial immune complex to form between the antigen and the conjugate antibody. This complex then migrates to the capture layer (which contains immobilized capture antibody) to form a complete immune complex “sandwich.” Unbound material is directed away from the capture zone via the channels in the wash layer. After a predetermined period of time following sample addition, the device is peeled in such a way to reveal the capture layer where the results are read.

The same concept can be applied to detection of an amplified polynucleotide product on the sliding strip device of our invention. For example, the detected antigen would be comprised of either an antigen incorporated into the amplicon itself or a labeled probe bound to the amplicon in a previous step. Movement of the sliding strip would bring the test zone into contact with dried antibody particles comprising the detection reagent. The antibody and antigenic labeled polynucleotide product would then form a complex via incubation either in the test zone or via release into a dwell region. Antigen capture would be followed by fluid movement of the antibody-antigen/amplicon complex into a capture layer comprised of bound antibody that also recognizes an antigen within the amplified polynucleotide product or a probe bound to the amplicon. Unbound detection reagent particles could be directed to a wash channel reservoir, leaving behind only detection reagent complexed to amplicons captured by surface-bound antibodies of the capture layer. In another embodiment of the invention, amplicons could be captured via binding to complementary nucleotide sequences bound to the surface rather than surface-bound antibodies. Release of amplicons from the test zone may be accomplished either by introduction of release agents (e.g., Tris-HCL/EDTA buffer) added manually to the test zone via an inlet or activation of dried reagents disposed in the device that can be activated upon contact with liquid in the test zone.

Conjugate Layer Design and Composition in Antibody-Mediated Capture—

In a preferred embodiment, the test zone encounters a dried formulation of antibody conjugate comprising a conjugate layer when registered at the appropriate station. In some embodiments, the antibody conjugate is mixed with a stabilizer prior to deposition onto the porous substrate. The stabilizer serves to readily release the antibody conjugate into solution upon contact with a fluid sample. In preferred embodiments, the stabilizer is selected from, but not limited to, the following: trehalose, sucrose, mannose, glucose, poly(ethylene glycol), poly(vinyl alcohol), poly(vinyl pyrrolidone), gelatin, dextran, albumin, poly(ethylene imine), silk, casein, and arabinogalactan.

Dwell Layer Design and Composition in Antibody-Mediated Capture—

In certain embodiments, the device contains a dwell layer which serves to provide a pre-determined incubation time for a solution at a particular point in the device. For example, it may be useful for an antibody conjugate and an antigen present in the sample to incubate before coming in contact with a capture antibody. The dwell layer takes the form of a patterned zone where the hydrophilic, porous zone contains a hydrophobic material designed to slow the wicking rate of a fluid. In one embodiment, the hydrophobic material is wax. The wax can be printed onto the dwell layer using the same printer (Xerox Phaser 8560) that is used to create the hydrophobic barriers. The barriers are typically printed using a black color in a graphic design program. Varying amounts of wax can be printed into the dwell zone by using the grayscale available in most programs, such as Adobe® Illustrator. The printer generates a gray color by simply printing varying percentages of black wax ink against the white paper background. Thus, by simply selecting a particular shade of gray which typically ranges from 1 to 99% black one can control the amount of wax that is deposited into a particular zone. In this way, one can vary the time it takes for fluid to pass through this semi-hydrophobic zone by increasing the intensity of the grayscale in that zone. Delay times can vary from a few seconds to hours using this method.

In some embodiments, the dwell region can be fabricated by depositing solutions containing varying amounts of hydrophobic materials. In preferred embodiments these solutions contain polymers such as polystyrene or waxes such as paraffin. In some embodiments, the solution may contain between about 0.001% and about 0.01% hydrophobic material, between about 0.01% and about 0.1% hydrophobic material, between about 0.1% and about 1% hydrophobic material, between about 1% and about 10% hydrophobic material, between about 10% and about 50% hydrophobic material, or between about 50% and about 100% hydrophobic material. Any suitable solvent can be used to form the solution.

In still further embodiments, the slightly hydrophobic zones can be created by depositing solutions containing varying amounts of hydrophobic materials. In preferred embodiments, these solutions contain polymers such as polystyrene or waxes such as paraffin. Such methods have been described by Phillips et al. in Anal. Chem., 2010, 82, 8071-8078 to generate timers but have not been used to enhance incubation times in an immunoassay.

In still further embodiments, the dwell layer can take the form of a channel of defined length. The length of the channel is proportional to the time it takes for a fluid to travel the distance of the channel. Thus, a fluid sample containing antigen that encounters a conjugate antibody will have an incubation time corresponding to the length of the channel. Upon reaching the end of this channel, the fluid will travel vertically to a capture zone to form a full immune complex. In some embodiments, it may be useful for this channel to also contain hydrophobic materials to slow the wicking speed even more. These materials can be deposited in the same manner as described above using a wax printer or solution. In further embodiments, the channel's width is a key variable in determining dwell time. For example, a channel may start wide, then narrow for a portion and then widen, resulting in a lower flow rate at the narrow portion of the channel. In some embodiments, the channel's flow path may influence the dwell time. For example, the channel may have a serpentine flow path, where, for example, the number of turns and/or the length of the turns of the flow path can be adjusted to control the dwell time.

Capture Layer Design and Composition—

In certain embodiments, the device contains a capture layer. The material for the capture layers is selected from, but not limited to: nitrocellulose membrane, nylon membranes such as Immunodyne® and Biodyne® membrane sold by Pall®, paper, chromatography paper, and non-woven polymeric membranes. In some embodiments, the material may be chemically treated to enhance or decrease protein binding. In some embodiments, the material may be treated with a surfactant, such as to enhance wettability.

In preferred embodiments, the capture layer contains a capture antibody which can bind to an antigen present in a given sample. The capture antibody is typically applied via an aqueous solution and dried to adsorb the antibody to the capture zone. It is often useful to bake the antibody after drying to enhance the conformation of the antibody. In preferred embodiments, a “blocking agent” is applied after deposition of the capture antibody. The blocking agent serves to prevent non-specific protein adsorption to the capture zone. The blocking agent may be selected from, but not limited to, the following: casein, bovine serum albumin, mouse Immunoglobulin G, poly(ethylene glycol), Tween®, Pluronic®, or Zwittergent® or polyvinyl alcohol. In some embodiments, the capture layer contains a capture oligonucleotide comprised of a nucleotide sequence complementary to part of the amplified polynucleotide product sequence.

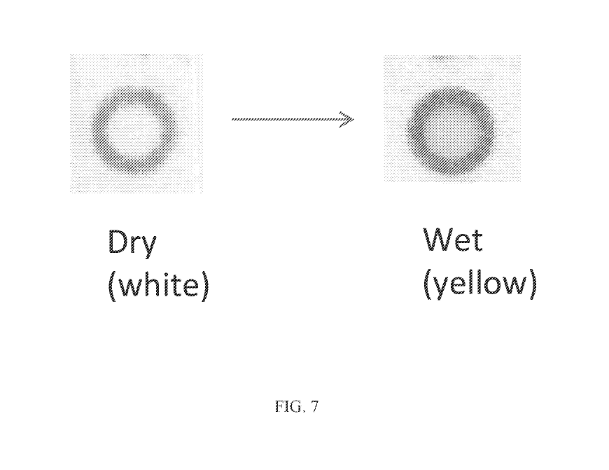

In some embodiments, a colored ink can be used to enhance the contrast of a colorimetric readout within the capture zone. An example of this is seen in FIG. 6 where a yellow ring of colored wax ink is used to enhance a blue signal output. In still further embodiments, a background color can be incorporated that is revealed when the capture zone is wet (FIG. 7). This can serve to enhance the ability of a user to interpret gradations of color. For example, a zone with a yellow background that accumulates blue latex particles will appear yellow if no particles are bound, green if low levels of particles are bound and blue if high levels are bound.

Control Region—

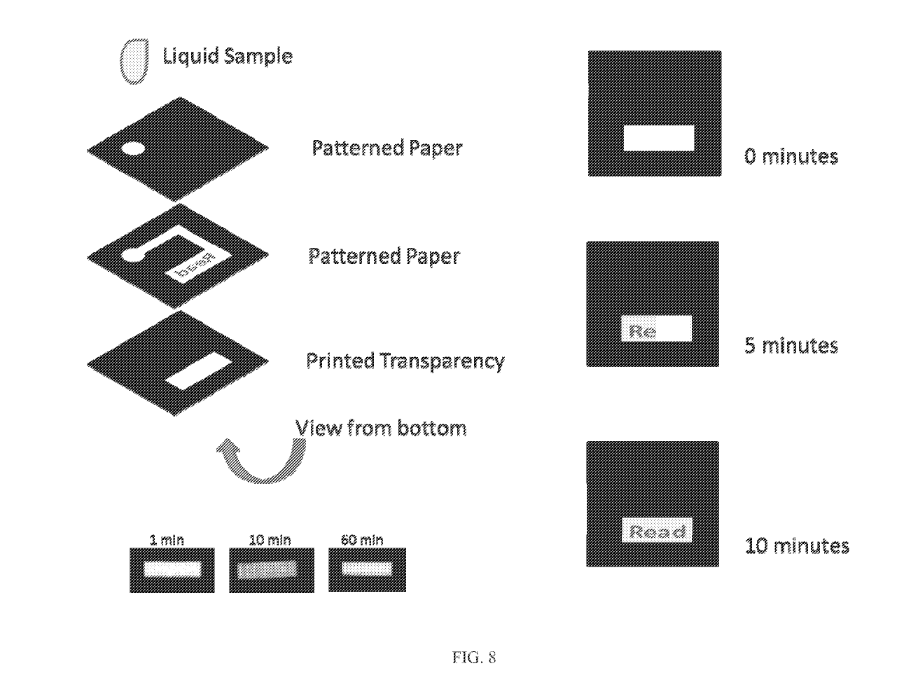

In some embodiments, accurate assessment of device function and colorimetric readout of the detection reagent may be aided by comparison to a control region. A control region can change color upon wetting, for example, to indicate device activation. In some embodiments, this effect may be achieved using a pigment on a layer of the test device. For example, the pigment may not be visible from the side opposite of the side on which the pigment is printed when the device is in a dry state. Without wishing to be bound by any theory, it is believed that the pigment is essentially not visible when the device is in the dry state due to the scattering of light by the fibers (e.g., cellulose) in the porous, hydrophilic sheet and the difference in refractive index between the fibers and the air. Upon introduction of fluid into the porous, hydrophilic sheet, the refractive index difference is reduced and the porous, hydrophilic sheet becomes semi-transparent, thus revealing the colored pigment on the reverse side. This simple effect is further illustrated in FIG. 8 and can serve two important functions. Firstly, observation of color change from white to a color other than white, e.g., yellow or another background color, can indicate to the user that a sufficient volume of fluid sample has been applied to the device and wicked to the appropriate region. Secondly, the color may serve as a background color to add contrast to a given colorimetric reaction.

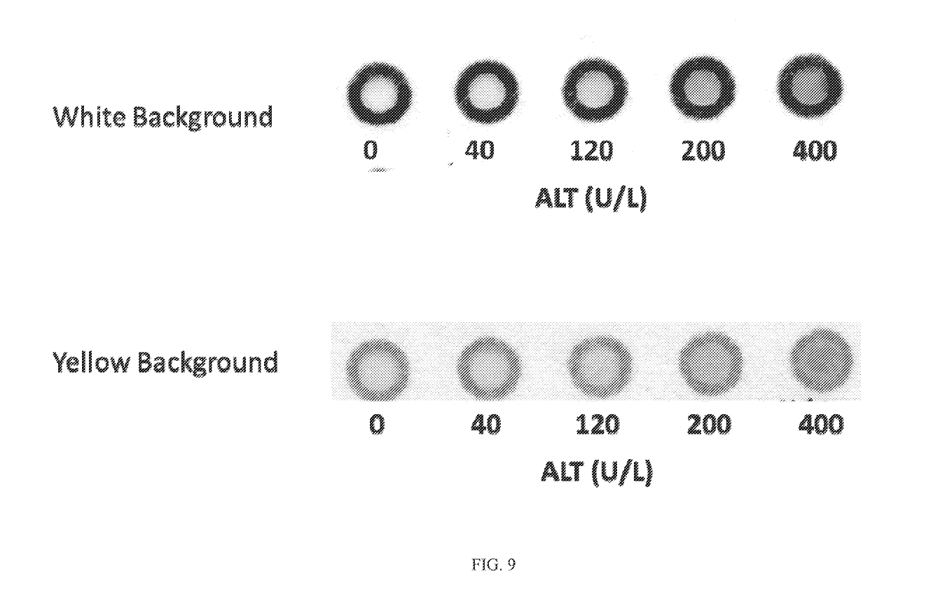

An example of the color adding contrast is shown in FIG. 9, where an assay which results in the production of a red/purple-colored dye progresses through shades of red/purple with increasing analyte concentration when performed on a white background (top panel), whereas this same reaction progresses from yellow to orange to red when performed against a yellow background (bottom panel) thus resulting in different colors with changing concentration as opposed to varying shades of the same color with changing concentration. Advantageously, this effect can greatly aid in the ability of a user to interpret colorimetric data. Also advantageously, the color can reverse back to white when the functional region is dry, thereby indicating to a user that a device is past the window for when it can be read and valid results obtained.

Timer—

A timer may be incorporated into the device which serves to indicate to an operator when the device should be read or when the sliding member should be moved to the next station. Such timers have been described by Phillips et al. in Anal. Chem., 2010, 82, 8071-8078, which is incorporated herein by reference in its entirety. In further embodiments, a timer takes the form of a multi-layer device containing a channel of defined length and width such that fluid takes a predictable amount of time to travel to the end of the channel. Upon initiation of contact between the test zone and a station comprising a timer, fluid disposed in the test zone or manually added to an inlet at the timer station immediately begins to wick down the defined paper channels. As the fluid wets the channel, it can reveal printed messages on the reverse side of the paper as the paper becomes wet, and therefore transparent. This concept is illustrated in FIG. 8. In some embodiments, a timer of this type could be incorporated in a test device by incorporating a split layer at the appropriate station where the fluid then travels to both the test zone and the timer channel simultaneously. In one embodiment of the device, a timer can indicate when an amplification step is completed and the sliding member can be moved to the next station in the device. In yet another embodiment, the timer can indicate when a detection step has been completed.

In certain embodiments, the positive control can act as a timer for the test in that when the positive control is fully developed, the device can be read. In further embodiments, the assay may be sensitive to heat or humidity leading to an acceleration or deceleration of the assay. In this situation, a positive control can be tailored such that it exhibits the same acceleration or deceleration effect. In this way, the device may be still read when the positive control is developed.

Multiple Output Zone Format—

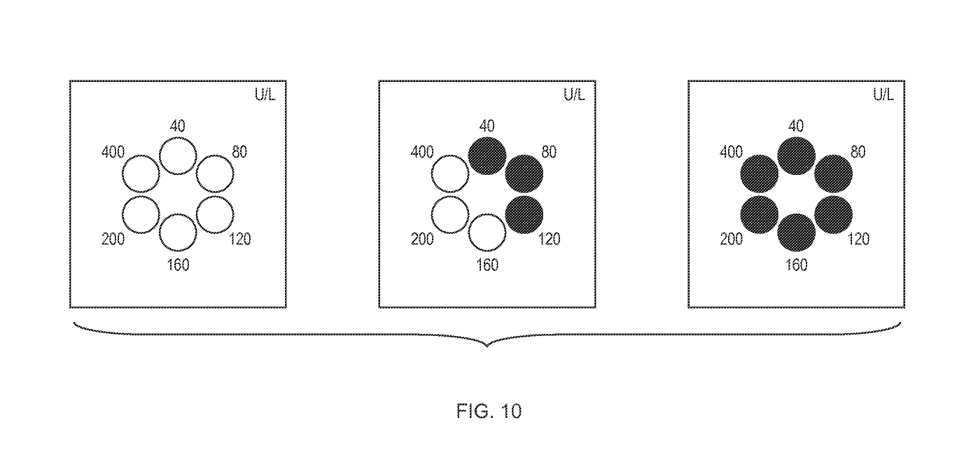

In another embodiment, a test device comprises multiple output zones. Each zone may be spotted with the same reaction chemistry but in progressively higher concentrations. The concentrations may be chosen such that increasingly higher levels of analyte may be needed to induce a color change in each zone. Thus, the number of zones “activated” will correlate to the amount of analyte in a given sample, resulting in a quantitative readout. For example, a surface capture antibody may be spotted at progressively higher concentrations in multiple zones such that higher levels of labeled analyte will result in activation and color change in output zones spotted with progressively higher levels of surface capture antibody. An illustration of this embodiment is shown in FIG. 10. For example, in a six zone readout, a sample with normal concentration would have no zones displaying color (FIG. 10, panel A); at elevated concentrations, zones 1-3 would show color (FIG. 10, panel B); and at highly elevated concentrations, all 6 zones would show color (FIG. 10, panel C).

“Plus and Minus” Readout—

Visual results can take the form of a “plus” sign “+” or “minus” sign “−”. This is accomplished by having a horizontal control line crossed by a vertical sample line. Lines can be generated by printing capture antibodies using plotters, inkjet printers, etc. In this way, a sample which is negative for a particular analyte will only activate the control line and develop as negative minus “−” symbol while a sample which contains a particular analyte will develop both the horizontal and vertical lines and reveal a plus “+” symbol.

Readout by a Cellular Device—

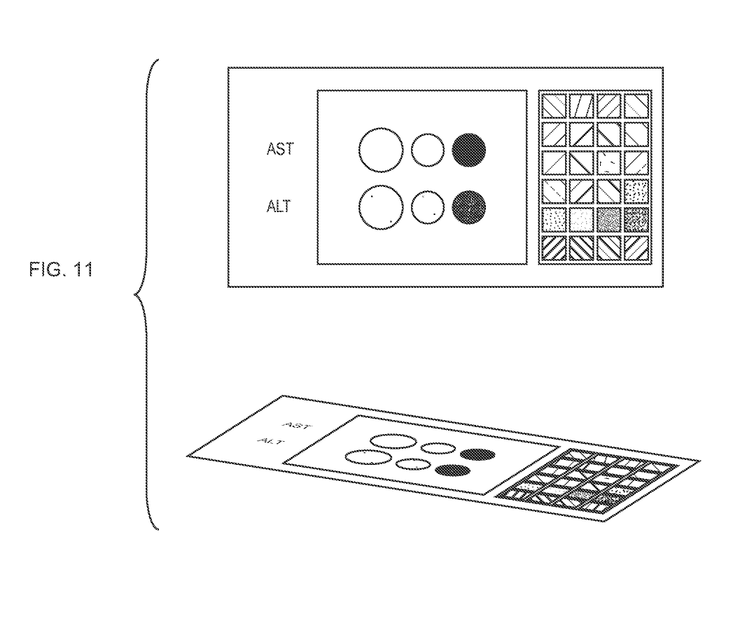

The colorimetric output of the device may be read and interpreted using a cellular phone. Using color intensity analysis software to interpret results enables one to achieve extremely high resolution—even approaching that of an automated method. In addition, interpretation of colorimetric data by this method provides other advantages such as automating inclusion of results in an electronic medical record and facilitating easy transmission for medical decision-making. A telemedicine application would also obviate any concerns about color-blind users. A further embodiment of the current invention is the use of cellular phones and accompanying software to meet the following requirements: (i) the system must work on a basic camera phone (such as those common to the developing world); (ii) data gathered by the camera must not be sensitive to camera angle, lighting, or distance from the lens (in preferred embodiments, the paper device contains a color chart which the phone software is able to use for automated calibration (FIG. 11)); and (iii) the system should be able to automatically recognize the pattern of test zones on the device to minimize user burden. In further embodiments, the device used to record the image is not a cell phone but any device capable of reflectance-based measurement and transmission.

### Multiplexing

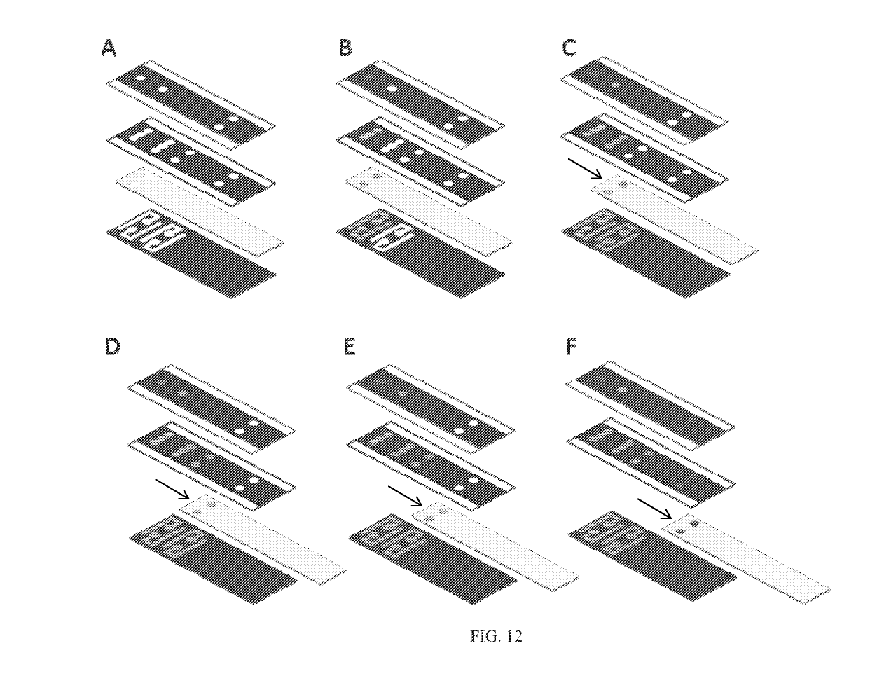

It is possible to build multiplexed devices (capable of detecting more than one analyte simultaneously) using the format described herein. FIG. 12 illustrates a device comprising five stations capable of detecting two analytes. In Part A, the device contains four layers comprising four planar members. The uppermost layer serves as the sample receiving layer and the layer directly below the uppermost layer serves to both split the sample into separate channels as well as store reagents for amplification and detection. The layer directly below the latter layer contains two sample discs, each containing lysis/capture chemistry. This layer is able to slide relative to the other layers such that said discs can be registered at each of the defined station within the device and interact with components thereof. The bottommost layer contains wash channels which act to wick away excess fluid.

In one embodiment, the device works as follows: B) a sample is introduced into the first station where it is filtered and the resulting plasma wicks to the split layer where it is divided into two separate paths. The plasma then wicks vertically to the sample discs where lysis/adsorption of the target material occurs. Finally, the plasma wicks through the wash channel and the entire volume is pulled through. C) Next, the sliding member is moved relative to the other planar members such that the reaction discs are brought into contact with the second station. A drop of wash buffer is introduced to the second station where it is split into two paths which then wick through the respective sample discs to a second set of wash channels which act to absorb the entire volume of wash buffer. D) The strip is then further slid to a third station where the wet discs can dissolve dried amplification reagents for each respective assay. In one embodiment, reagents for malaria could be added to one disc and reagents for dengue fever added to another. E) The strip is slid to a fourth station where the discs are hermetically sealed and heating/amplification can occur. F) The strip is then slid to a fifth station where the solution in the discs is allowed to contact a colorimetric reagent stored in the device. It is of note that both tests can be performed entirely independent of the other in this device and therefore issues related to primer interactions between the two assays are avoided. Finally, it will be apparent to those skilled in the art in view of this specification that a similar device design can be used to multiplex more than two tests. In theory, tens or even hundreds of zones could be independently addressed.

### “In Plane” Sliding Strip Design

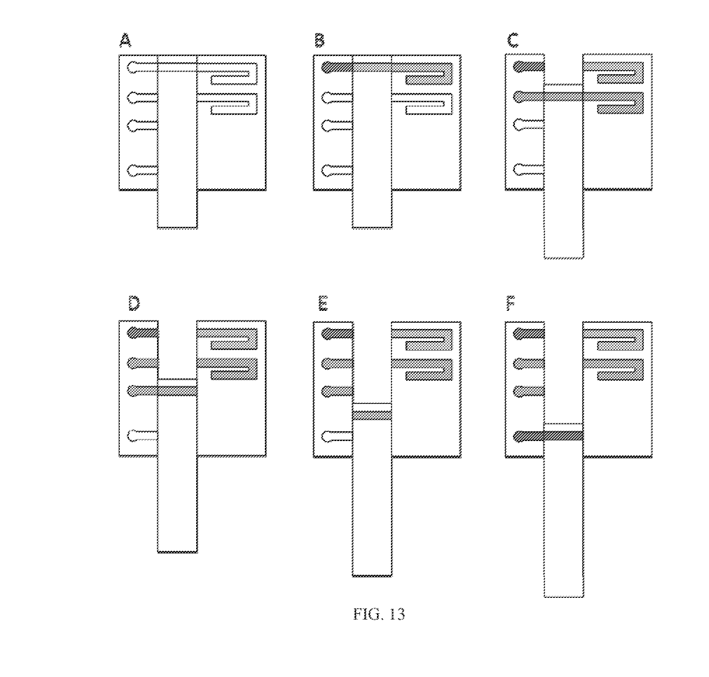

FIG. 13 illustrates a particular embodiment of the device where the sliding strip lies and moves within the same plane relative to other portions of the device. A) The device consists of three substantially planar members. On one end is a first planar member comprising sample/buffer loading regions (leftmost member in FIG. 13A). Adjacent to this component is a second strip containing a channel where lysis/adsorption can occur (middle member in FIG. 13A). Adjacent to the latter component is a third planar member (rightmost member, FIG. 13A) positioned such that the second planar member is situated between the other two members and contacts the inner edge of each of the other two planar members of the device. The third planar member contains wash channels capable of making fluidic contact with the middle strip channel. In a hypothetical example, the device would work as follows: B) a sample is introduced into the first station of the first planar member where it is filtered and the resulting plasma wicks to the channel on the middle strip layer where lysis/adsorption of the target material occurs. Plasma then wicks through the wash channel in the third member, and the entire volume is pulled through. C) The strip is slid to the second station where a drop of wash buffer is introduced and then wicks to the wash channel on the third member which acts to absorb the entire volume of wash buffer, thereby purifying the sample. D) The strip is then slid to a region where the wet channel can dissolve dried amplification reagents. E) The strip is further slid to a region where the channel region is hermetically sealed and heating/amplification can occur. F) The strip is then further slid to a region where the solution in the strip channel is allowed to contact a colorimetric reagent stored in the fourth station of the device.

### Automated Casing

In some embodiments, a disposable sliding strip device may be placed into a handheld casing. The casing may be battery operated and serve to automate movement of the sliding planar member and heating process used to drive the amplification reactions. Digital readout devices capable of interfacing with analytical instrumentation located within the handheld casing are contemplated.

Further Description of Exemplary Devices with a Colorimetric Readout

As described above, analytical devices containing a colorimetric readout provide an easy way to communicate the results of an analytical detection. Provided below is additional description of exemplary analytical devices containing a colorimetric readout. It is understood that the polynucleotide detection reagents and procedures described herein can be incorporated into the colorimetric analytical devices described below and/or features of the devices and methods described below can be incorporated into the devices and methods described above.

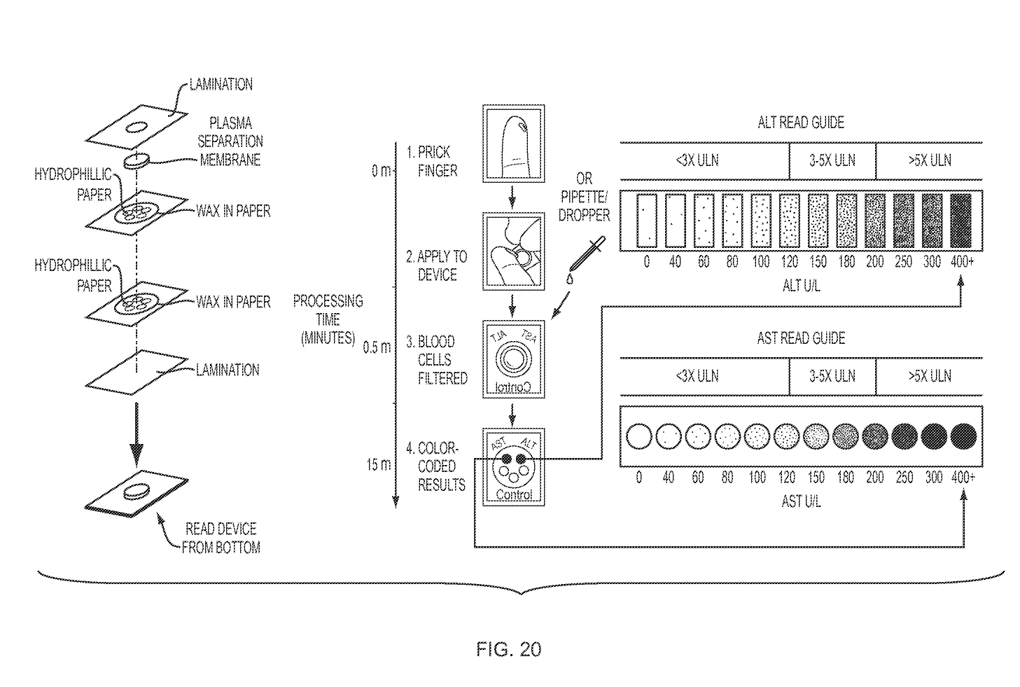

One type of colorimetric assay device uses an aspartate aminotransferase (AST) and/or alanine aminotransferase (ALT) testing protocol. Referring to FIG. 20, a non-limiting exploded view of an aspartate aminotransferase (AST)/alanine aminotransferase (ALT) test device and an exemplary assay protocol are shown. A test device may comprise a plurality of sheets (i.e., layers) disposed parallel to one another (e.g., to form a stacked configuration), as shown in panel A of FIG. 20. The device may include a plurality of porous, hydrophilic sheets, which may be disposed between hydrophobic sheets, such as a top laminate and a bottom laminate. The top-laminate includes a sample inlet defined by an opening in the top-laminate. The device may further include a filter (e.g., a plasma separation membrane) that, in some embodiments, may be positioned between the top laminate and a porous, hydrophilic sheet.

As shown in FIG. 20, the porous, hydrophilic sheets may be patterned with a hydrophobic barrier (e.g., wax) to form one or more functional regions (e.g., a sample input, a test readout, a positive control, a negative control, a flow path, and the like). In the exemplary test device shown in panel A, functional regions define two test regions and three control regions. One or more reagents may be deposited on one or both of the porous, hydrophilic sheets. The layers may be affixed to each other using, for example, an adhesive and/or by laminating the stacked layers.

Referring now to panel B of FIG. 20, a drop of biological fluid (e.g., blood) may be applied to the sample inlet of the test device. Cells in the biological fluid (e.g., erythrocytes and leukocytes) are separated by the filter in the device and the resultant plasma wicks through the functional regions. After a period of time (e.g., about 15 minutes) the test regions are compared to a corresponding color guide (FIG. 20, panel C) to quantify the results of the assay. In some embodiments, the results may be interpreted as being within range of values, e.g., less than about three times (<3×) the upper limit of normal (ULN, defined in this example as 40 U/L), between about three and about five times (3-5×) the upper limit of normal, or greater than five times (>5×) the upper limit of normal.

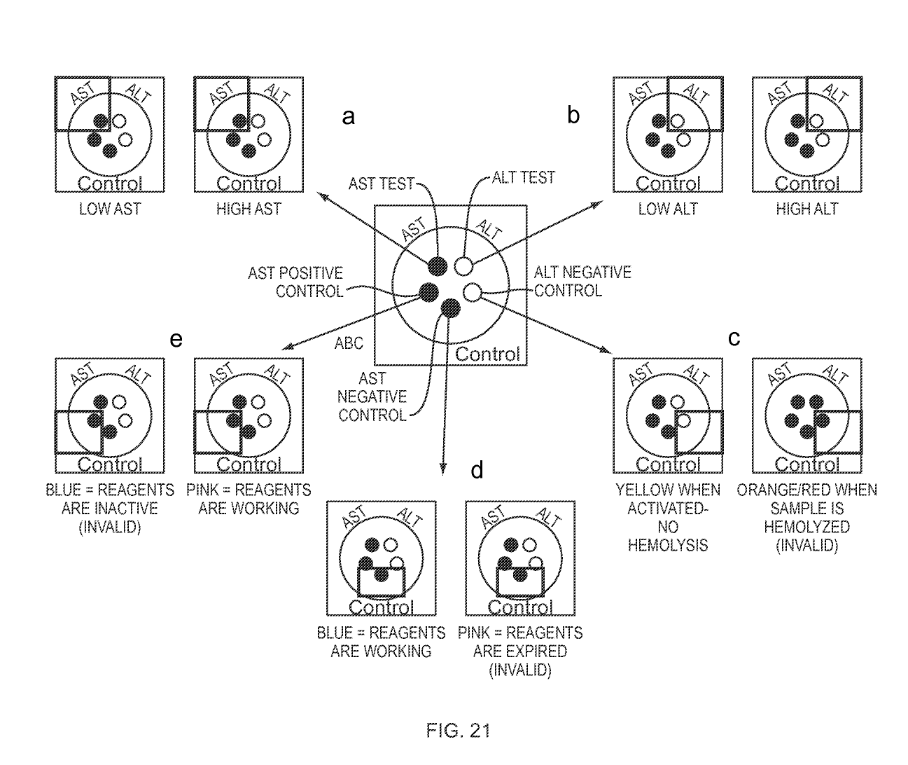

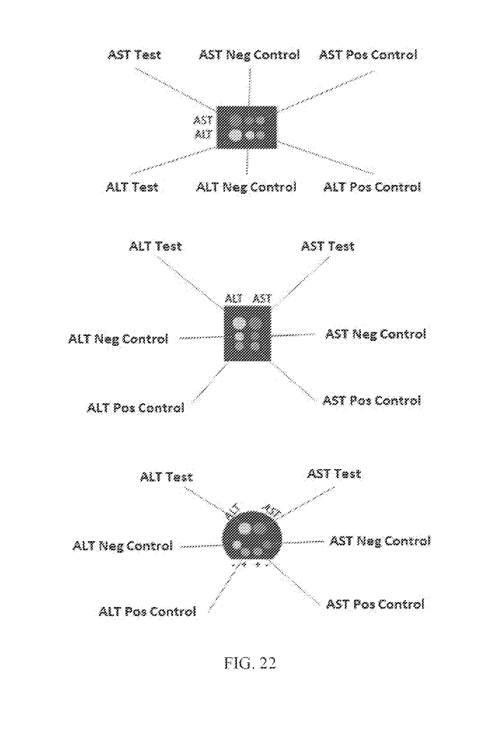

FIG. 21 further illustrates the use of a liver function test device and provides various readout possibilities. A schematic of test and control regions is shown in the center of the figure. In this exemplary device, an AST test, an AST positive control, an AST negative control, an ALT test, and an ALT negative control are provided. As shown in panel A, in the AST test region, normal AST values (e.g., <80 units/Liter (U/L)) result in a dark blue color (“Low AST”), whereas high AST values (e.g., >200 U/L) result in a bright pink color (“High AST”). In the ALT test region (as shown in panel B), normal ALT values (e.g., <60 units/Liter (U/L)) result in a yellow color (“Low ALT”), whereas high ALT values (e.g., >200 U/L) result in a deep red color (“High ALT”). Panels C, D, and E illustrate the operation of control regions in the test device. In the ALT negative control region (panel C), a change from white to yellow occurs upon wetting of the region, indicating appropriate device activation and essentially no hemolysis (“Yellow when activated—no hemolysis”), whereas in the event of sample hemolysis, the region becomes orange/red and the device is read as “invalid” (“Orange/red when sample in hemolyzed (invalid)”). In the AST negative control region (panel D), the baseline blue color remains unchanged if dye chemistry is functioning properly (“Blue=reagents are working”), whereas the control region becomes bright pink in the event of non-specific dye reaction (“Pink=reagents are expired (invalid)”) and the device is read as “invalid.” In the AST positive control region (panel E), the region changes from blue to pink if AST reagents are functioning properly (“Blue=reagents are inactive (invalid)”), but remains dark blue if either the reagents are not functioning or the zone is not activated (“Pink=reagents are working”), and the device is read as “invalid.”

As shown in FIG. 21, panel C, a control region can change color upon wetting, for example, to indicate device activation. In some embodiments, this effect may be achieved using a pigment on a layer of the test device. For example, the pigment may not be visible from the side opposite of the side on which the pigment is printed when the device is in a dry state. Without wishing to be bound by any theory, it is believed that the pigment is essentially not visible when the device is in the dry state due to the scattering of light by the fibers (e.g., cellulose) in the porous, hydrophilic sheet and the difference in refractive index between the fibers and the air. Upon introduction of fluid into the porous, hydrophilic sheet, the refractive index difference is reduced and the porous, hydrophilic sheet becomes semi-transparent, thus revealing the colored pigment on the reverse side. This simple effect is further illustrated in FIG. 7 and can serve two important functions. Firstly, observation of color change from white to a color other than white, e.g., yellow or another background color, can indicate to the user that a sufficient volume of fluid sample has been applied to the device and wicked to the appropriate region. Secondly, the color may serve as a background color to add contrast to a given colorimetric reaction. An example of the color adding contrast is shown in FIG. 9, where an ALT assay which results in the production of a red/purple-colored dye progresses through shades of red/purple with increasing ALT concentration when performed on a white background (top panel), whereas this same reaction progresses from yellow to orange to red when performed against a yellow background (bottom panel) thus resulting in different colors with changing concentration as opposed to varying shades of the same color with changing concentration. Advantageously, this effect can greatly aid in the ability of a user to interpret colorimetric data. Also advantageously, the color can reverse back to white when the functional region is dry, thereby indicating to a user that a device is past the window for when it can be read and valid results obtained.

In some embodiments, it is particularly useful to have two or more layers of patterned paper in the device. For instance, with two or more layers, separation of reagents that would otherwise react quickly when mixed may be achieved. For example, in the device positive controls, a first layer of paper may contains dried enzyme (e.g., AST or ALT) and the second layer may contain reagents (e.g., substrates) that react with the enzyme. This configuration may operate as follows. A sample may be added into the device, and fluid from the sample wicks into the first layer, releasing the dried enzyme, and then to the second layer where the enzymes can mix with the reagents (e.g., reactive chemistry). By contrast, in some cases, if the enzyme was deposited on the same layer as the reactive chemistry, it could react prematurely leading to undesired results. Separation of reagents into different layers also can allow for separate formulation chemistry to be used to stabilize specific reagents. For example, an enzyme could be stabilized with a sugar in one layer, and a dye molecule stabilized with a water-soluble polymer in another layer. In addition, multi-layer devices can help prevent migration of dyes or other reagents, which is often seen when flow occurs only in a lateral direction.

In a preferred embodiment, the liver transaminase test may contain six test zones. This design provides a test zone for ALT with separate positive and negative controls and a test zone for AST with separate positive and negative controls. Various designs and layouts can be considered for the zones. FIG. 6 illustrates some non-limiting potential designs for six zone tests.

A particularly useful chemistry for measurement of AST and ALT in a blood sample are known AST and ALT assays. The AST assay chemistry utilizes AST present in a sample to convert cysteine sulfinic acid and alpha-ketoglutaric acid to L-glutamic acid and beta-sulfinyl pyruvate. The beta-sulfinyl pyruvate reacts with water to yield free SO₃⁻ which further reacts with methyl green, a blue-colored dye, to yield a colorless compound. This reaction is performed against a pink contrast dye, created by also spotting Rhodamine B onto the paper. As the reaction proceeds, and the dye becomes converted to a transparent compound, more of the pink background is revealed. The visual result is that the detection zone changes from a dark blue to a bright pink color in the presence of AST.

The ALT assay chemistry is based on the conversion by ALT of L-alanine and alpha-ketoglutaric acid to pyruvate and L-glutamic acid, the subsequent oxidation of pyruvate by pyruvate oxidase to form acetyl phosphate and hydrogen peroxide, and the utilization of the liberated hydrogen peroxide by horseradish peroxidase to generate a red-colored dye 4-N-(1-imino-3-carboxy-5-N,N dimethylamino-1,2-cyclohexanediene) through the coupling of 4-amino antipyrine and N,N-dimethylaminobenzoic acid. In further embodiments, the pyruvate generated in the AST chemistry could be used in the same reaction cascade as in the ALT assay as described in U.S. Pat. No. 5,508,173.

Huang et al. describe several methods for transaminase detection in Sensors 2006; 6(7):756-782, which is hereby incorporated by reference in entirety. Additionally, Anon et al. describe methods for AST and ALT detection in Scand. J. Clin. Lab. Invest. 1974; 33(4):291-306, which is hereby incorporated by reference in entirety.

In further embodiments, it is envisioned that additional zones could be added to the test device to accommodate more assays. In an notional embodiment, the test contains detection zones for ALT, AST, bilirubin, ALP, GGT, and albumin along with positive and negative controls for some or all of the tests. In still further embodiments, the AST and ALT assays may be multi-plexed with other assays such as creatinine for monitor of kidney function or even immunoassays such as those used to detect hepatitis.

While various aspects of the test device have been exemplified in the context of liver function tests, it should be understood that the test device is not limited to liver function tests. Any suitable biological assay may be performed using the test device described herein. For example, the biological assay may be used to quantify a component of a biological fluid, such as a protein, nucleic acid, carbohydrate, peptide, hormone, small molecule, virus, cell, microorganism, and the like. The biological assay may also be used to quantify an activity (e.g., blood clotting, ALT, AST, amylase, creatine kinase, etc.) in a biological fluid.

In some embodiments, the multiple layers of a test device may be held together by an adhesive. Any suitable adhesive may be used. For example, in some instances, a hydrophobic, polymeric, adhesive may be used. In further embodiments, the adhesive may be patterned by a printing technique including, but not limited to, screen printing, flexographic printing, gravure printing, transfer printing, and ink jetprinting. A preferred embodiment is to pattern the adhesive by screen printing. Whitesides et al. report a method for adhering multiple layers of patterned paper together using double-sided tape cut with a laser cutter (Proc Natl Acad Sci 105:19606-19611, which is incorporated herein by reference in entirety). When the cut double-sided tape is used, it leaves a gap caused by the thickness of the tape and prevents contact between the hydrophilic regions of the patterned paper. This gap must be filled with cellulose powder to enable z-direction flow (i.e., tangential flow through the device). Screen printing of adhesives offers several advantages over this technique. For example, the patterned adhesive layer typically can be applied in very small thicknesses (e.g., between about 1 and about 500 microns, between about 1 and about 100 microns, between about 1 and about 50 microns, and between about 50 and 100 microns), which allows for intimate contact to occur between the hydrophilic regions of the patterned paper and eliminates the need to use the cellulose powder filler. Screen printing may also require much less material than double-sided tape, which reduces device raw material cost. Furthermore, screen-printing is a low-cost and easily scaled patterning technique, which is advantageous for inexpensive, mass production of the test devices. In the specific embodiment of the paper Liver Transaminase test, the printed adhesive holds the paper in contact as well as ensures contact to the plasma separation filter through adhesion. In a preferred embodiment, the adhesive may be a pressure sensitive adhesive. In further preferred embodiments, the adhesive is Unitak 131 sold by Henkel Corporation.

The manufacturing unit operations for a test device can be separated into a series of steps. For example, in some embodiments, the manufacturing operations may include some or all of the following steps: patterning of the paper substrate with hydrophobic barriers, patterning of adhesive by screen printing, deposition of biological/chemical reagents, layer alignment and assembly, attachment of plasma separation membrane, and/or lamination and packaging.

A preferred method for patterning paper to be used in a test device is wax printing, although any suitable method for creating hydrophobic barriers on a porous, hydrophilic sheet may be used. Wax printing is described in detail by Whitesides et al. in Anal Chem 81:7091-7095 and International Patent Application Publication No. WO 2010/102294, both of which are hereby incorporated by reference in entirety. The device may be designed on a computer and the hydrophobic walls of the microfluidic channels may be printed onto a sheet of paper using a commercial printer with solid-ink technology (e.g., using a Xerox Phaser printer). The printer generally operates by melting the wax-based solid ink and depositing the ink on top of the paper. The sheet is then heated to above the melting point of the wax, allowing wax to permeate through the thickness of the paper, thereby creating a hydrophobic barrier through the entire thickness of the paper. In some cases, spreading of the wax may occur during the heating step, but the spreading is reproducible based on the type of paper used and the thickness of the printed line and can be incorporated into the design. Without wishing to be bound by any theory, it is believed that the channels patterned in the paper wick microliter volumes of fluids by capillary action and distribute the fluids into test zones where independent assays can take place.

Other method embodiments may use paper soaked in photoresist which is then exposed to UV light through a photomask with a desired pattern. The unexposed regions are then washed away with a suitable solvent, leaving behind crosslinked hydrophobic regions that penetrate the thickness of the paper. Feature sizes as small as 100 μm have been demonstrated using this technique. Examples of this method of patterning can be found in prior work from in Angew. Chem. Int. Ed. 2007, 46, 1318-1320 and International Patent Application Publication No. WO 2008/049083, which is hereby incorporated by reference in entirety. In further embodiments, there is a host of other large-scale printing and patterning techniques that can be used to deposit hydrophobic barriers into paper to meet the requirements of the test device. These methods include, but are not limited to: screen-printing, gravure printing, contact printing, flexographic printing, hot embossing, ink jet printing, and batik printing.

In several embodiments of the present invention, the layers may be adhered together in such a way that fluids can wick in the z-direction (i.e., tangentially) to entry points in the next layer of paper. One method of accomplishing this is by using double-sided adhesive tape with holes cut into the desired pattern through which fluid can flow. This method is described in more detail in Proc. Natl. Acad. Sci. USA, 2008, 105, 19606, which is hereby incorporated by reference in entirety. In this particular method, a hydrophilic powder (i.e., cellulose) may be added in the cut aperture between the layers of paper formed by the thickness of the tape. A preferred method for assembly of 3-D devices is to use simple and scalable screen-printing techniques to deposit very thin layers of adhesive onto paper in the desired pattern. In this manner, a hydrophobic, pressure-sensitive adhesive (e.g., Unitak 131 sold by Henkel Corporation) can be applied to the paper. Once adhesive is applied, pre-made sheets can be stored by laminating the adhesive side to a non-adhesive release layer, for example as commonly seen in other adhesive products such as labels and tapes. In further embodiments, a stencil can be fabricated and pressed against a sheet of patterned paper in such a way that certain features are covered. An adhesive may then be deposited from an aerosol spray onto the remaining exposed regions.

In preferred embodiments, it is necessary to deposit chemical and/or biological assay reagents into regions of the device. The reagents react with analytes present in a bodily fluid and which yields a response (i.e., colorimetric or electrochemical) that can indicate the concentration of a particular analyte. In some embodiments, it is often necessary to formulate reagents with appropriate stabilizers (e.g., sugars) to preserve function once dried. In one embodiment, useful for prototyping and small scale production (e.g., 100's of devices per day), deposition of reagents is done by hand using micropipettes and repeat pipetters. A typical volume deposited is between 0.5 and 5 μL. In preferred embodiments for larger scale production, precision liquid deposition machines can be used. Two examples of such tools are the AD3400 available from BioDot, Inc. and the Diamatix DMP-2800 Ink Jet printer available from Fujifilm. Both of these units are able to rapidly dispense precise volumes (contact-free) of fluid down to nL volumes in a programmed pattern. Additionally, such units can be adapted to continuous manufacturing lines for large scale production.

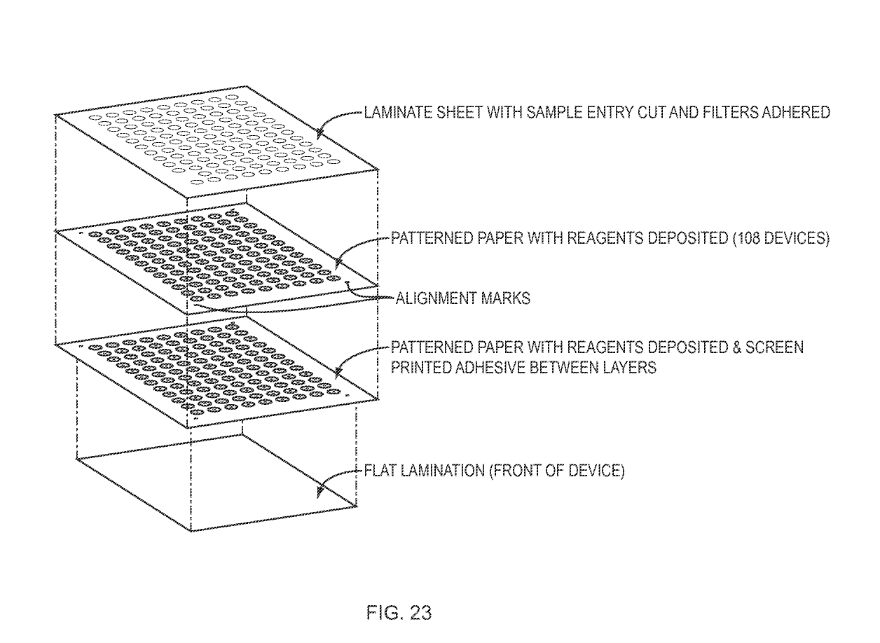

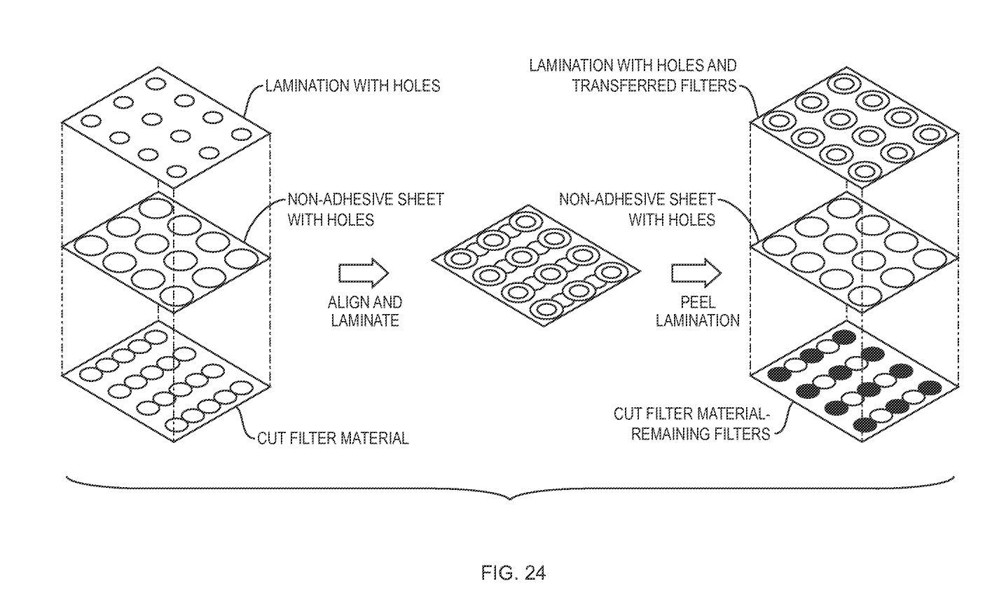

In preferred methods of manufacture, devices are assembled in full sheets, for example, as shown in FIG. 23. For this to occur, it is imperative that patterned regions precisely align to make the necessary fluidic junctions possible between layers. A simple and scalable way to accomplish this is to cut precise holes in the paper layers such that the sheets can slide onto peg boards. Each layer can then be applied to the peg board such that features are rapidly aligned correctly. The adhesive applied earlier acts to lock the sheets in place once in contact. In continuous manufacturing, a similar method can be used on reels containing pegs such as that used in Dot-Matrix Printing. Alternatively, laser web guides can be used to precisely align sheets before lamination. Other methods for aligning the sheets will be known to those of ordinary skill in the art.

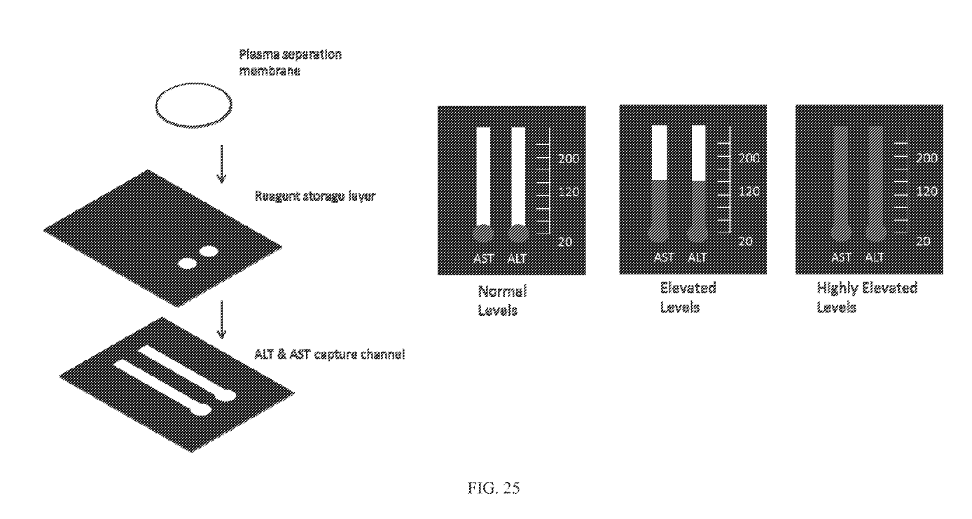

As seen in FIG. 20, a plasma separation membrane (Pall Corporation) may be placed at the entry point of the device. The membrane may serve as a reservoir to collect a biological fluid (e.g., a blood drop) and importantly to filter cells (e.g., red blood cells) out of the biological fluid and allow fluid (e.g., plasma) to wick into the device zones. Accordingly, certain embodiments utilize a “pick and place” method consisting of the following steps (illustrated in FIG. 25): (i) a sheet of Pall membrane may be cut into densely packed circles 1 cm in diameter using a laser cutter or die cutter. The cut sheet may be laminated to a surface with low adhesion such as a low-tack laminate sheet or a rubbery sheet. In preferred embodiments, the cut membrane sheet is adhered to a PET film coated with PDMS; (ii) a sheet of adhesive laminate may be cut using a knife plotter, laser cutter, die cutter, or the like such that it contains apertures which act as an entry point into the filter/device (top layer of FIG. 23). The holes in the laminate sheet may be between about 0.1 cm and about 1.5 cm in diameter or between about 0.5 cm and 1.0 cm. In a preferred embodiment, the holes are about 0.75 cm in diameter; (iii) a non-adhesive masking layer may be cut, e.g., from waxy cardstock, or other materials with low adhesion, in a pattern to have holes that are larger than the filters. For example, in some embodiments, the diameter of the holes in the non-adhesive masking layer may be more than about 0.2 cm, more than about 0.3 cm, more than about 0.4 cm, or more than about 0.5 cm larger than the diameter of the holes in the membrane. In a preferred embodiment, the holes in the masking layer are about 1.13 cm in diameter; (iv) the previously cut laminate containing 0.75 cm holes and the masking layer may be adhered together such that the laminate aperture is in the middle of the blocking layer aperture; (v) the stack may be placed over the densely cut membrane sheet in such a way as to only pick up filter membrane discs that align with the cut laminate sheet. The others membrane discs are blocked by the masking layer; (vi) the stack may be then laminated and the adhesive laminate layer peeled away which, as it is peeled, adheres a filter over each laminate aperture on the laminate sheet while leaving the others behind for the next set of devices; and (vii) the laminate layer, now with a filter membrane adhered under each aperture, may be adhered to a stack of two layers of patterned paper which may be adhered together by screen printed adhesive. In this way, the maximum area of the membrane material can be converted into useable filtration discs for devices. Using dye-cutting techniques and simple laminators, this process can be easily automated into large scale-production.

After the steps above have taken place, the stack of patterned paper (and filters, etc., if required) may be laminated. In some embodiments, a “cold lamination” sheet consisting of a PET film with adhesive on one side may be used. The film protects the devices and provides the outer hydrophobic layer for the patterned zones. The device elements may then be separated into separate devices (e.g., cut into separate devices). In some embodiments, the devices may be placed in foil-lined bags and heat sealed, preferably where the bags contain a desiccant.

In some embodiments, it is useful to have certain sample handling features built into the device itself. For example, one such feature is a simple plastic cover that protects the sample entry aperture. After a drop of biological fluid is introduced to the device via the entry aperture and into the filter membrane, a plastic cover may then seal the aperture to slow the evaporation and drying of the fluids in the device.

In further notional embodiments, it may be desired to have a built-in capillary capable of drawing a precise volume of blood into the device by simply making contact with the droplet. Such a feature can minimize user operations and ensures reproducibility in the volume of sample introduced to the device.

In still further notional embodiments, a test device may contain a built-in lancet, which is disposed of along with the device after use.

In some embodiments, the device may be used as part of a kit containing a glass or plastic capillary tube; in preferred embodiments the tube is plastic, such as the MicroSafte Tube available from Safe-Tec®. In some embodiments, the kit may contain a lancet; in preferred embodiments, the lancet is a spring-loaded lancet, such as those available from Surgilance™. In still further embodiments, a kit will contain patterned paper devices, a lancet, a capillary tube, a bandage, an alcohol swab, latex gloves, and a colorimetric read guide for interpretation of results.

As discussed above, in some embodiments, a filter may be incorporated into the device that serves to filter out blood cells (as well as dirt, fibers, etc.) for the isolation of plasma, which then wicks into the device. In preferred embodiments, the filter is a Vivd™ membrane available from Pall corporation. In other embodiments, the membrane can be a glass fiber membrane, or even a paper filter. In other embodiments, anti-blood cell antibodies may be attached to the membrane to facilitate capture of cells. In further embodiments, “scrubbing agents” may be added to the filter membrane or paper channels that are capable of capturing substances that may interfere with the reaction chemistry.

Nearly any porous material can be patterned by the methods disclosed. Accordingly, many materials can be patterned to generate a liver function test according to the present invention. Materials include, but are not limited to: paper, chromatography paper, nitrocellulose, non-woven polymeric materials, lab wipes, nylon membranes such as Immunodyne® membranes sold by Pall® corporation. A preferred material for the present invention is Whatman® no. 1 chromatography paper.

In some embodiments, stabilizers may be added to the reagent zones to further stabilize the enzymes spotted onto the paper. In further embodiments the stabilizers, include but are not limited to, Trehalose, Poly(ethylene glycol), Poly(vinyl alcohol), Poly(vinyl pyrrolidone), Gelatin, Dextran, Mannose, Sucrose, Glucose, Albumin, Poly(ethylene imine), Silk, and Arabinogalactan. In some embodiments, dye stabilizers such as MgCl₂ or ZnCl₂ may be added to the assays.

In preferred embodiments, the stabilizers are sugars. A particularly useful method for stabilizing enzymes and other proteins, vacuum foam drying, is described by Bronshtein et al. in U.S. Pat. No. 6,509,146, which is incorporated herein by reference in entirety.

In some embodiments, a timer may be incorporated into the device which serves to indicate to an operator when the device should be read. Such timers have been described by Phillips et al. in Anal. Chem., 2010, 82, 8071-8078, which is incorporated herein by reference in entirety. In further embodiments, a timer takes the form of a multi-layer device containing a channel of defined length and width such that fluid takes a predictable amount of time to travel to the end of the channel. Upon addition of sample to the device, fluid immediately begins to wick down the defined paper channels. As the fluid wets the channel, it can reveal printed messages on the reverse side of the paper as the paper becomes wet, and therefore transparent. This concept is illustrated in FIG. 8. In some embodiments, a timer of this type could be incorporated in a test device by incorporating a split layer after the entry where the fluid then travels to both the test zone and the timer channel simultaneously.

In certain embodiments, the positive control can act as a timer for the test in that when the positive control is fully developed, the device can be read. In further embodiments, the assay may be sensitive to heat or humidity leading to an acceleration or deceleration of the assay. In this situation, a positive control can be tailored such that it exhibits the same acceleration or deceleration effect. In this way, the device may be still read when the positive control is developed.

In some embodiments, the device may contain a dwell region which serves to provide a pre-determined incubation time for a solution at a particular point in the device. For example, it may be useful for an antibody conjugate and an antigen present in the sample to incubate before coming in contact with a capture antibody. The dwell region may take the form of a patterned zone where the hydrophilic, porous zone contains a hydrophobic material designed to slow the wicking rate of a fluid. In a preferred embodiment, the hydrophobic material is wax. The wax can be printed onto the dwell region using the same printer that is used to create the hydrophobic barriers (e.g., a Xerox Phaser 8560). In some instances, the barriers may be printed using a black color in a graphic design program. Varying amounts of wax can be printed into the dwell region by using the grayscale feature available, for example, in computer illustration programs, such as Adobe® Illustrator. In some embodiments, the printer generates a gray color by simply printing varying percentages of black wax ink against the white paper background. Thus, by simply selecting a particular shade of gray which can range, for example, from about 1% to about 99% black, one can control the amount of wax that is deposited into a particular zone. In this way, the time it takes for fluid to pass through the dwell region can be varied by increasing the intensity of the grayscale in the dwell region. Delay times can vary from a few seconds to hours using this method. For example, the delay time may be between about 1 second and about 5 seconds, between about 2 seconds and about 10 seconds, between about 5 seconds and about 15 seconds, between about 10 seconds and about 30 seconds, between about 15 seconds and about 1 minute, between about 30 seconds and about 2 minutes, between about 1 minute and about 5 minutes, between about 2 minutes and about 10 minutes, between about 5 minutes and about 20 minutes, between about 10 minutes and about 30 minutes, between about 20 minutes and about 1 hour, between about 30 minutes and about 2 hours, between about 1 hour and about 3 hours, between about 2 hours and about 4 hours, and the like.

In still further embodiments, the dwell region can be fabricated by depositing solutions containing varying amounts of hydrophobic materials. In preferred embodiments, these solutions contain polymers such as polystyrene or waxes such as paraffin. In some embodiments, the solution may contain between about 0.001% and about 0.01% hydrophobic material, between about 0.01% and about 0.1% hydrophobic material, between about 0.1% and about 1% hydrophobic material, between about 1% and about 10% hydrophobic material, between about 10% and about 50% hydrophobic material, or between about 50% and about 100% hydrophobic material. Any suitable solvent can be used to form the solution.

In still further embodiments, the dwell region can take the form of a channel of defined length. The length of the channel may be proportional to the time it takes for a fluid to travel the distance of the channel. Thus, for example, a fluid sample containing antigen that is introduced to a device and mixed with a conjugate antibody may have an incubation time corresponding to the length of the channel. Upon reaching the end of this channel, the fluid may travel vertically to a capture zone to form a full immune complex. In some embodiments, it may be useful for this channel to also contain hydrophobic materials to slow the wicking speed even more. These materials can be deposited in the same manner as described above using a wax printer or solution. In further embodiments, the channel's width may influence the dwell time. For example, a channel may start wide, then narrow for a portion and then widen, resulting in a lower flow rate at the narrow portion of the channel as compared to the wide portion of the channel. In some embodiments, the channel's flow path may influence the dwell time. For example, the channel may have a serpentine flow path, where, for example, the number of turns and/or the length of the turns of the flow path can be adjusted to control the dwell time.

In another embodiment, a multi-layer device formed from patterned paper is shown in FIG. 25. This particular design allows for a quantitative colorimetric readout. The device comprises a plasma separation membrane adhered to one or more layers of patterned paper comprising regions (i.e., zones) used to store reagents which are formulated to release upon contact with fluid sample. The ALT zone may contain L-alanine, alpha-ketoglutaric acid, pyruvate oxidase, horseradish peroxidase, 4-amino antipyrine, and N,N-dimethylaminobenzoic acid. The AST zone may contain cysteine sulfonic acid, alpha-ketoglutaric acid and methyl green dye. The layers of patterned paper may be adhered to a bottom layer consisting of patterned channels. The channels in this design may have anti-ALT and anti-AST antibodies immobilized to the paper fibers that form the channels. In this way, a blood sample may be introduced to the filter membrane, wick down to the two reagent zones where reagents for each assay are released from the paper, and then begin to wick down the corresponding channels. As the sample (now containing reagents) wicks down the channel, the AST or ALT may be captured by the antibodies. The more ALT or AST present in the sample, the further down the channel it will be present as it is captured. In this manner, the colorimetric reaction will only proceed in the presence of ALT or AST and therefore will yield a “thermometer” type readout whereby higher amounts of ALT or AST will give color further down the channel. Theoretical outcomes are shown in FIG. 25 for normal, elevated, and highly elevated levels of AST and ALT.

In another embodiment, a test device comprises multiple output zones. Each zone may be spotted with the same reaction chemistry but in progressively higher concentrations. The concentrations may be chosen such that increasingly higher levels of analyte may be needed to induce a color change in each zone. Thus, the number of zones “activated” will correlate to the amount of analyte in a given sample, resulting in a quantitative readout. An illustration of this embodiment is shown in FIG. 10. For example, in a six zone readout, a sample with normal concentration would have no zones displaying color (FIG. 10, panel A); at elevated concentrations, zones 1-3 would show color (FIG. 10, panel B); and at highly elevated concentrations, all 6 zones would show color (FIG. 10, panel C).

In some embodiments, the colorimetric output of the device may be read and interpreted using a cellular phone. While the liver function test will have high utility when read by eye, using color intensity analysis software to interpret results enables one to achieve extremely high resolution—even approaching that of an automated method. In addition, interpretation of colorimetric data by this method provides other advantages such as automating inclusion of results in an electronic medical record and facilitating easy transmission for medical decision-making. A telemedicine application would also obviate any concerns about color-blind users. A further embodiment of the current invention is the use of cellular phones and accompanying software to meet the following requirements: (i) the system must work on a basic camera phone (such as those common to the developing world); (ii) the data gathered by the camera must not be sensitive to camera angle, lighting, or distance from the lens. In preferred embodiments, the paper device contains a color chart which the phone software is able to use for automated calibration (see FIG. 11); and (iii) the system should be able to automatically recognize the pattern of test zones on the device to minimize user burden. In further embodiments, the device used to record the image is not a cell phone but any device capable of reflectance-based measurement and transmission.

An exemplary five-zone device can be fabricated as follows:

### Materials for ALT Assay:

Alanine Solution: A solution containing 1M L-alanine (Sigma Aldrich), 30 mM alpha-ketoglutaric acid (Sigma Aldrich), 2 mM KH₂PO₄ (Sigma Aldrich), 20 mM MgCl₂ (Sigma Aldrich), 2 mM Thiamine Pyrophosphate (MP Biosciences), 2 mM of 4-aminoantipyrine (Sigma Aldrich) and 25 U/mL (0.1 mg/mL) Horseradish Peroxidase (HRP) (Sigma Aldrich) was prepared in 200 mM Tris buffer (pH=7.4). DABA Solution: A solution containing 10 wt % PEG (MW=35,000 g/mol, Sigma Aldrich) and 10 mM Dimethylaminobenzoic acid was prepared in DI water. Pyruvate Oxidase: A solution containing 100 U/mL of Pyruvate Oxidase (MP Biosciences, EMD) was prepared in 200 mM Tris buffer pH=7.4. PEG Solution: A solution containing 5 wt % PEG (MW=35,000 g/mol, Sigma Aldrich) was prepared in DI water.

### Materials for AST Assay:

PVA Solution: A solution containing 2 wt % of PVA (87-90% Hydrolyzed, MW=13,000-23,000 g/mol, Sigma Aldrich) and 0.05% of Triton X 100 (Sigma Aldrich) was prepared in DI water. Tris Buffer (400 mM): A solution of 4.8456 g Tris Base (Sigma Aldrich) in 100 mL DI H₂O (pH=8.0) was prepared. EDTA: A 10 mL solution containing 0.75 g EDTA (Sigma Aldrich) in 400 mM Tris Buffer and the pH was adjusted to 8.0. Phosphate Buffer (40 mM): A 100 mL solution containing 0.038 g NaH₂PO₄.H₂O (Sigma Aldrich), 1 g Na₂HPO₄.7H₂O (Sigma Aldrich), and 0.387 g of NaCl was prepared and the pH was adjusted to 8.0. Methyl green Solution: A 1.2% solution of methyl green was prepared by dissolving 0.6 g of methyl green into 50 mL of the PVA solution (prepared above). Rhodamine B Solution: A 1.2% solution of Rhodamine B was prepared by dissolving 0.6 g of Rhodamine B into 50 mL of the PVA solution (prepared above). AST Dye Solution: A solution containing 0.6% Methyl Green and 0.05% Rhodamine B in 1% PVA was prepared by combining 600 μL of methyl green solution with 100 μL of rhodamine B solution and 500 μL of 1% PVA solution. CSA Solution: 171.1 mg CSA (Sigma Aldrich), 14.6 mg alpha-ketoglutaric acid and 10 μL of 200 mM EDTA solution was prepared in 1 mL of 40 mM Phosphate Buffer and the pH was adjusted to 8.0. AST Positive Control Solution (200KU/L AST solution, 5% PEG, in 1× PBS): A solution was prepared containing 5% PEG (MW=35,000 g/mol, Sigma Aldrich) in 1× PBS and 6.17 μL AST (5177 U/mL, MP Biosciences) were added to make 200 KU/L AST solution. This step was done immediately prior to device fabrication.

### Procedures for Device Fabrication:

Device patterns were designed using Adobe Illustrator CS3. A sheet of Whatman No. 1 chromatography paper (8.5×11″) was fed into a laser printer (HP Color Laserjet 4520) and yellow stripes were printed on the back of the sheet to align with the ALT zones. A wax pattern for the top layer (layer from which the device is read) of devices was printed onto this sheet using a Xerox 8560DN printer such that the wax was printed on the opposite side of the yellow stripe. The sheet was heated in the oven at 150° C. for 30 seconds to ensure the wax migrated through the thickness of the paper. A wax pattern for the bottom layer of devices (layer which receives filters) was printed onto Whatman No. 1 Chromatography paper using a Xerox 8560DN printer. This sheet was also heated in an oven at 150° C. for 30 s to ensure the wax migrated through the thickness of the paper.

A pressure-sensitive adhesive (UNITAK 131, Henkel) was applied to the back of the top layer by screen printing. The printing screen was patterned using known methods with photocurable emulsion (Atlas Screen Printing Supply) such that the 5 active zones of the device did not receive adhesive but the remaining areas did. The layer was placed in an oven set at 70° C. for 15 min to drive off water from the adhesive leaving behind a patterned, tacky layer of adhesive with “holes” over the zones. This screen-printing process was repeated on the back of the bottom layer. The sheets were then taped to a plastic frame in order to spot reagents.

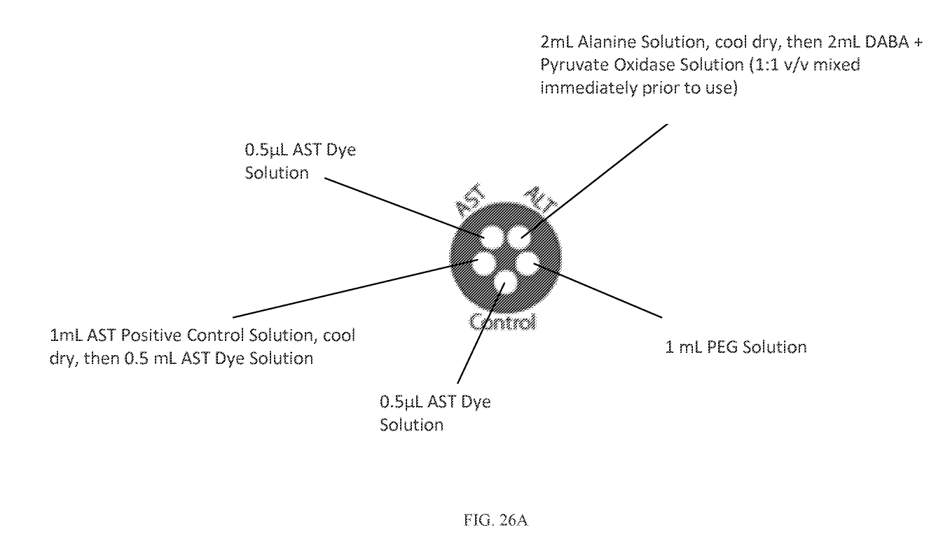

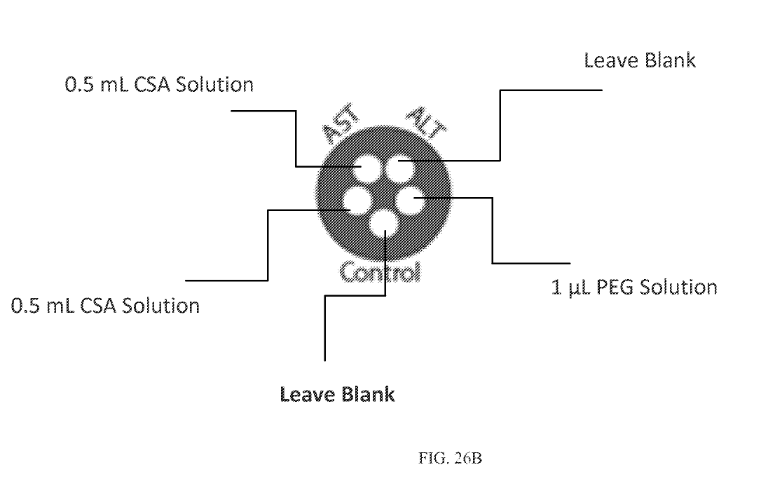

Zones were spotted using a micropipette according to FIGS. 26A and 26B. If multiple spots were required, the first spot was allowed to dry completely (air dry at room temperature) before applying the second.

A hole-puncher was used to punch alignment holes (pre-printed on the corners of each sheet) in both device layers. Device layers were aligned by aligning the previously punched holes. The aligned layers were then sandwiched between two non-adhesive waxy sheets and passed through a laminator at a speed of 2 ft/min. Cold lamination (Fellowes self-adhesive laminate sheets) was then placed on the front face of the sheet of devices. A second sheet of Fellows laminate was cut or punched with 7 mm holes and placed on a bench adhesive side up. 1 cm pre-cut discs of Pall Vivid GX plasma separation membrane were then centered over the holes in the laminate sheet in such a way that the rough side of the membrane was in contact with the adhesive. This process was repeated until each device had a corresponding filter. The cut laminate with adhered filters was then aligned and laminated to the back of the device sheet stack such that each filter covered all 5 zones of the device. Finally, the entire stack was laminated a total of 8 times (4 times with each side facing up) to ensure good contact. Individual devices were then cut by hand and stored in heat-sealed foil-lined bags containing 1 packet of silica desiccant with 10 devices/bag.

Additional exemplary colorimetric devices include devices comprising a porous, hydrophilic sheet, e.g. adsorptive paper or nitrocellulose, defining plural functional regions including a liquid sample input; a colorimetric test readout; a negative control that upon absorption of the sample maintains or displays a predetermined color; a positive control, and a liquid flow path which, responsive to application of a liquid sample to the input, transports liquid between the input and both the readout and controls. Disposed in the device, e.g., adjacent the input region or in the test region, or in a reagent reservoir in fluid communication with the liquid flow path, is at least one dried, color-producing reagent arranged to produce a shade or pattern of color in a readout as a function of the concentration of an analyte in the sample. Also disposed in the device is a dried, color-producing reagent which react at the positive control to produce color. In these embodiments of devices of the invention, a valid test is indicated by only if there is a color change in the positive control and maintenance or display of a predetermined color at the negative control.

Further provided is a family of test devices for quantitative determination of an analyte in a liquid biological sample which have elements in common with the embodiment described in the previous paragraph, but the colorimetric test readout includes a region of a color backing the readout, e.g., a region of printed color, which optically interacts with color developed at the readout to improve visual discrimination among different analyte concentrations in an applied sample. Thus, this type of device comprises a porous, hydrophilic sheet defining plural functional regions including a liquid sample input; a colorimetric test readout including the region of a color backing the readout which optically interacts with color developed at the readout; a colorimetric control; and a liquid flow path which transports liquid between the input and both the readout and the control. Again, disposed in the device is a dried, color-producing reagent which, responsive to application of a liquid sample to the input, is entrained and reacts with an analyte, if present in the applied sample, to produce a visually detectable change of color (as opposed to an intensity of a single color) in the readout as a function of the concentration of an analyte in the sample.

In certain embodiments, the device comprises a plurality of sheets disposed parallel to one another, e.g., stacked or laminated, at least two of which are separated by a liquid impermeable barrier layer defining an opening permitting liquid flow communication between the sheets. The color producing reagent may react with any analyte, and in one preferred embodiment, reacts with one or more liver enzymes to detect pathologic liver function such as elevated levels or concentrations of aspartate aminotransferase, alanine aminotransferase, or a mixtures thereof. The negative control may comprise a colored area applied to a sheet which has an appearance when wetted different from when dry. The readout may comprise an area of a sheet comprising immobilized binder which captures a colored species produced by the color-producing reagents. This permits display or a readout of the concentration of analyte in a sample as a portion of the area that develops color responsive to application of liquid to said input. The area may be continuous so that the concentration of analyte in a said sample is indicated, as in a mercury thermometer, by the linear extent of color development in the area. Alternatively, the area comprises a plurality of separate areas and the concentration of analyte in the sample is indicated by the number of areas that develop color.

In other embodiments, the device further comprises a region defining a timer comprising a reservoir disposed in the device in liquid communication with the inlet which, after application of a sample, receives liquid from the sample over a predetermined time interval and comprises indicia that the reservoir is filled and the device is ready to be read. The timer may for example comprise a channel of predefined dimensions which determines the length of time that liquid takes to reach the reservoir and to activate the indicia, which may comprise a printed message visible when the device is ready to be read. The timer also may function as a positive colorimetric control. Often, the timer is disposed downstream from the readout. Many of the devices of the invention comprise a filter disposed upstream of the inlet, e.g., to exclude colored components such as red blood cells or hemoglobin from transport through the flow structure of the device and to permit unhindered colorimetric readout.

In yet additional embodiments, the device further comprises downstream of the color-producing reagent and upstream of the colorimetric test readout, a dwell region which transports therethrough a mixture of analyte from a sample and the color-producing reagent, the dwell region comprising a multiplicity of micro flow paths including hydrophobic flow impeding surfaces, the numbers and dimensions of the micropaths serving to set the incubation time within a predetermined time interval as the mixture passes therethrough. The dwell region may be, for example, impregnated with a hydrophobic material (e.g., wax) which impedes the rate of liquid passage through the dwell region. In some cases, the dwell region is manufactured by printing a hydrophobic material onto a surface of a sheet and heating to absorb the hydrophobic material into the pores of the sheet.

In some embodiments, the device may comprise an adsorptive reservoir downstream of the colorimetric test readout for drawing liquid along the flow path and through the dwell region and colorimetric test readout thereby to remove unbound colored species from the colorimetric test readout. A device may comprise in some instances an immobilized binder (e.g., an antibody) at the colorimetric test readout for capturing a complex formed during incubation in the dwell region. The device may include a sheet holding a dried, color-producing reagent in fluid communication with a parallel disposed sheet defining the dwell region. In certain embodiments, at least two of the following elements of the device are defined on a single said adsorptive sheet: a region holding a dried, color-producing reagent; a sample inlet; a colorimetric test readout; a dwell region; and an adsorptive reservoir.

In three-dimensional embodiments, the devices may comprise a patterned layer of adhesive which constitutes the barrier layer between adjacent adsorptive or absorptive sheets and which defines an opening permitting liquid flow communication between the sheets. This provides flexibility and control, as well as multiplexing of test paths on a single device. For example, the inlet and readout may be disposed on different sheets, or the readout and a the color-producing reagent(s) may be disposed on different sheets

The devices may further comprise a color chart relating color at the readout to analyte concentration, and this may optionally be integrated with a sheet. Of course, plural readouts serviced by respective different dried, color-producing reagents are enabled by the disclosure herein. Flow paths in the devices typically comprises one or a pattern of hydrophilic channels which direct transport of liquid flow and are defined by liquid impermeable boundaries substantially permeating the thickness of the hydrophilic sheet. The devices optionally may include an electrode assembly comprising one or more electrodes in liquid flow communication with the input region, and/or a thermally or electrically conductive material in communication with a flow path which can serve to control flow as a valve, or to evaporate fluid, for example. See, for example, International Patent Application Publication No. WO/2009/121041 and U.S. Ser. No. 13/254,967, the disclosures of which are incorporated herein by reference.

Further provided are methods of manufacturing test devices for determination of one or more analytes in liquid biological samples enabling mass production of reliable, extremely inexpensive test devices designed for quantitative or semi-quantitative clinical assays for any one or combination of analytes. The method comprises the steps of a) providing a first porous, hydrophilic sheet which supports absorptive or adsorptive flow transport; b) printing onto the sheet an array of test device elements respectively comprising a pattern of hydrophobic barriers permeating the thickness of the porous sheet to define respective elements, each of which comprise plural functional regions including a liquid flow path and a colorimetric test readout; c) adhering to the first sheet a second porous, hydrophilic sheet to form a laminate; and d) cutting the laminate to separate individual elements to form a multiplicity of functional test devices. In preferred embodiments, prior to step d) one or more reagents are applied on each of the test device elements, e.g., by robotically pipetting. The reagents may be deposited on the first or second porous, hydrophilic sheet, or onto a separate structure that serves as a reagent reservoir located to be contacted with liquid sample applied to the input. The first and second sheets are aligned prior to step c to register structural features so as to implement fluid flow communication between the sheets. Also, the method may include the additional steps of providing a third sheet or additional multiple sheets defining other structure, e.g. an array of filter elements, and laminating the third or additional sheets to the first and second sheets to position functional structure such as a filter element in fluid communication with respected liquid flow paths of respective test device elements. Step c often comprises the step of providing a liquid impermeable layer between the first and second sheets, which may itself act as an adhesive layer. This may be done by application of two-sided adhesive sheet material designed to isolate flow of liquid on respective sheets except for one or more defined holes positioned to permit liquid flow communication between the sheets. Preferably, the liquid impermeable layer is produced by applying an adhesive to a sheet in a pattern.

Still further provided are methods of manufacturing further comprising applying by printing onto a region of the surface of a sheet a predetermined density of ink, causing the ink to penetrate the sheet, and hardening the ink to form a dwell region comprising a multiplicity of micro flow paths including hydrophobic flow impeding surfaces defined by the ink, the numbers and dimensions of the micropaths serving to set a predetermined time interval for liquid sample to pass through the dwell region. The method may further comprise the additional step of applying by printing onto the surface of the sheet a higher density of the ink to define a border of a flow path, causing the ink to penetrate the sheet, and hardening the ink to produce a liquid impermeable barrier defining a liquid flow path in fluid communication with the dwell region. Also, the method may include the additional step of laminating the sheet to at least one additional porous, hydrophilic sheet which supports absorptive flow transport, at least a portion of which is in liquid communication with the sheet, and which additional sheet defines at least one element selected from the group consisting of a flow path; a colorimetric test readout; an immobilized binder at a test region for capturing a complex; a second dwell region; a liquid sample inlet; a control site; a dried, color-producing reagent reservoir, an adsorptive reservoir, and a sample split layer. A sample split layer allows a sample to be divided, for example, so that multiple assays can be run in parallel.

The method may include yet another additional step of applying by printing onto the surface of the sheet a higher density of the ink to define a border of at least one element selected from the group consisting of a flow path; a colorimetric test readout; an immobilized binder at a test region for capturing a complex; a second dwell region; a liquid sample inlet; a control site; a dried, color-producing reagent reservoir; an adsorptive reservoir; and a sample split layer in liquid communication with the sheet, causing the ink to penetrate the sheet, and hardening the ink to produce a liquid impermeable barrier defining a border of the element. In some embodiments, method may comprise providing a filter or a color-producing reagent reservoir in fluid flow communication with the dwell region. The method may include applying by printing onto plural regions of the surface of the sheet in an array a predetermined density of ink to produce an array of the dwell regions, laminating the sheet to at least one additional porous, hydrophilic sheet which supports absorptive flow transport, at least a portion of which is in liquid communication with the sheet, and which additional sheet defines a corresponding array of at least one element selected from the group consisting of a flow path; a colorimetric test readout; an immobilized binder at a test region for capturing a complex; a second dwell region; a liquid sample inlet; a control site; a dried color-producing reagent reservoir; an adsorptive reservoir; and a sample split layer.

Exemplary Analytical Devices that can be Modified to Include Components for Detecting a Characteristic of a Polynucleotide Analyte

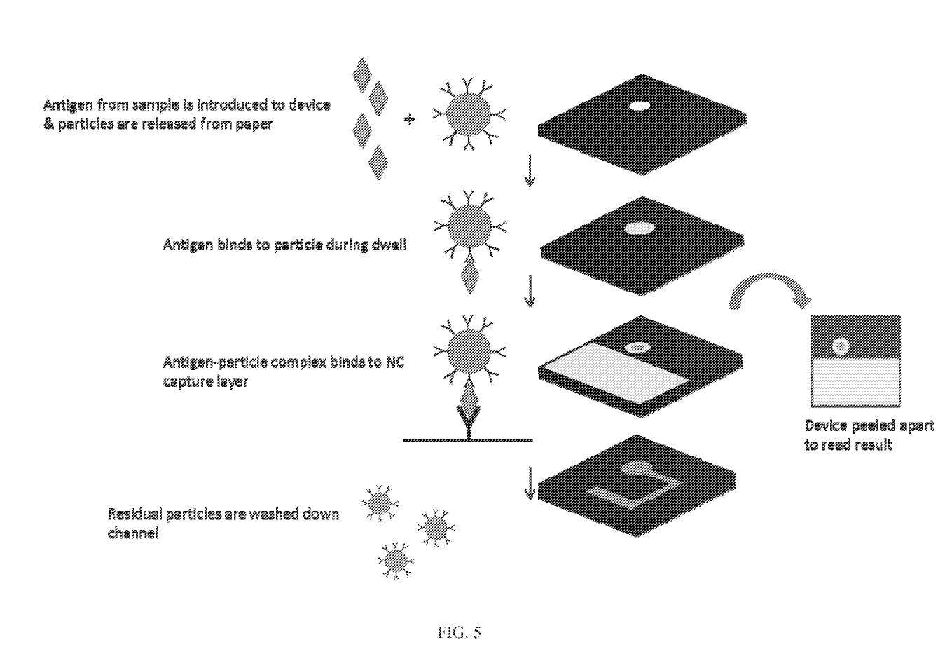

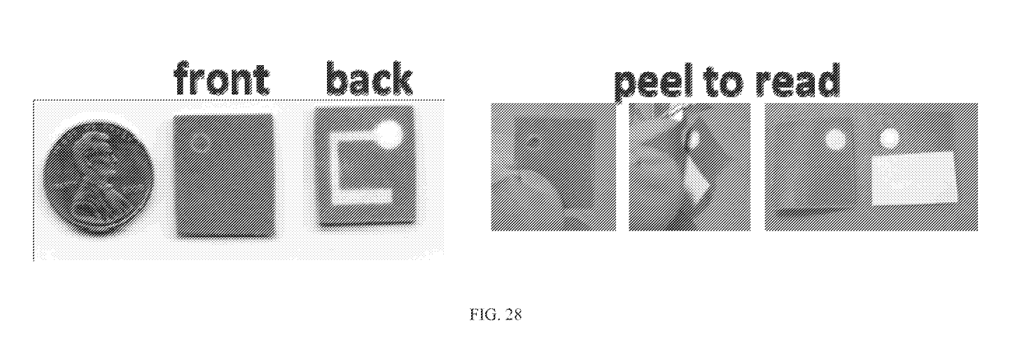

Devices containing four layers of paper patterned with hydrophobic wax which serve to define hydrophilic paths or zones are described in FIGS. 5, 27 and 28. The layers include i) a reagent storage layer, ii) a “dwell” layer, iii) a capture layer, and iv) a wash layer. In preferred embodiments, the layers are held together by screen-printed patterned adhesive layers. FIG. 5 illustrates the process steps performed in the device. A single drop of antigen-containing sample is placed into the top of the device, where it encounters the reagent storage layer, where antibody conjugates in the form of antibody-coated colored particles (such as latex or colloidal gold) have been previously spotted and formulated to release into solution once in contact with the liquid sample. The fluid sample then encounters the dwell layer, which is slightly hydrophobic but still permits wicking and acts to provide a programmed incubation time for a partial immune complex to form between the antigen and the conjugate antibody. This complex then migrates to the capture layer (which contains immobilized capture antibody) to form a complete immune complex “sandwich.” Unbound material is directed away from the capture zone via the channels in the wash layer. 5-20 minutes after sample addition, the device is peeled in such a way to reveal the capture layer where the results are read (FIG. 28).

### Conjugate Layer (Stabilizers, Etc.)

The conjugate layer may contain a dried formulation of antibody conjugate. In some embodiments, the antibody conjugate is in the form of an antibody linked to an enzyme. In certain preferred embodiments, the enzyme is horseradish peroxidase or alkaline phosphatase.

In further embodiments, the antibody conjugate is in the form of one or more antibodies linked to a colored particle. In some embodiments the particle is selected from, but not limited to, the following: a colored polymer latex particle, a colloidal gold particle, a graphite particle, a quantum dot, or a carbon nanotube.

In some embodiments, the antibody conjugate is mixed with a stabilizer prior to deposition onto the porous substrate. The stabilizer serves to readily release the antibody conjugate into solution upon contact with a fluid sample. In preferred embodiments, the stabilizer is selected from, but not limited to, the following: trehalose, sucrose, mannose, glucose, poly(ethylene glycol), polyvinyl alcohol), polyvinyl pyrrolidone), gelatin, dextran, albumin, poly(ethylene imine), silk, casein, and arabinogalactan.

### Dwell Layer (Printed Wax in Gray, Other Treatments, Dwell Channel)

The device may contain a dwell layer which serves to provide a pre-determined incubation time for a solution at a particular point in the device. For example, it may be useful for an antibody conjugate and an antigen present in the sample to incubate before coming in contact with a capture antibody. The dwell layer takes the form of a patterned zone where the hydrophilic, porous zone contains a hydrophobic material designed to slow the wicking rate of a fluid. In a particularly useful embodiment, the hydrophobic material is wax. The wax can be printed onto the dwell layer using the same printer (Xerox Phaser 8560) that is used to create the hydrophobic barriers. The barriers are typically printed using a black color in a graphic design program. Varying amounts of wax can be printed into the dwell zone by using the grayscale available in most programs, such as Adobe® Illustrator. The printer generates a gray color by simply printing varying percentages of black wax ink against the white paper background. Thus, by simply selecting a particular shade of gray which typically ranges from 1 to 99% black one can control the amount of wax that is deposited into a particular zone. In this way, one can vary the time it takes for fluid to pass through this semi-hydrophobic zone by increasing the intensity of the grayscale in that zone. Delay times can vary from a few seconds to hours using this method.

In still further embodiments, the slightly hydrophobic zones can be created by depositing solutions containing varying amounts of hydrophobic materials. In preferred embodiments these solutions contain polymers such as polystyrene or waxes such as paraffin. Such methods have been described by Phillips et al. in Anal. Chem., 2010, 82, 8071-8078 to generate timers but have not been used to enhance incubation times in an immunoassay.

In still further embodiments, the dwell layer can take the form of a channel of defined length. The length of the channel is proportional to the time it takes for a fluid to travel the distance of the channel. Thus, a fluid sample containing antigen that is introduced to a device and mixed with a conjugate antibody will have an incubation time corresponding to the length of the channel. Upon reaching the end of this channel, the fluid will travel vertically to a capture zone to form a full immune complex. In some embodiments, it may be useful for this channel to also contain hydrophobic materials to slow the wicking speed even more. These materials can be deposited in the same manner as described above using a wax printer or solution. In further embodiments, the channel's width is a key variable in determining dwell time. For example, a channel may start wide, then narrow for a portion and then widen, resulting in a lower flow rate at the narrow portion of the channel.

### Capture Layer (Materials, Surfactant Treatments, Blocking, Heating, Etc.)

The device may contain a capture layer. Material(s) for the capture layers is selected from, but not limited to: nitrocellulose membrane, nylon membranes such as Immunodyne® and Biodyne® membrane sold by Pall®, paper, chromatography paper, and non-woven polymeric membranes. In some embodiments, the material may be chemically treated to enhance or decrease protein binding. In some embodiments, the material may be treated with a surfactant, such as to enhance wettability.

In preferred embodiments, the capture layer contains a capture antibody which can bind to an antigen present in a given sample. The capture antibody is typically applied via an aqueous solution and dried to adsorb the antibody to the capture zone. It is often useful to bake the antibody after drying to enhance the conformation of the antibody. In preferred embodiments, a “blocking agent” is applied after deposition of the capture antibody. The blocking agent serves to prevent non-specific protein adsorption to the capture zone. The blocking agent may be selected from, but not limited to the following: casein, bovine serum albumin, mouse Immunoglobulin G, poly(ethylene glycol), Tween®, Pluronic®, or Zwittergent® or polyvinyl alcohol.

In some embodiments, a colored ink can be used to enhance the contrast of a colorimetric readout. An example of this is seen in FIG. 6 where a yellow ring of colored wax ink is used to enhance a blue signal output. In still further embodiments, a background color can be incorporated that is revealed when the capture zone is wet FIG. 7. This can serve to enhance the ability of a user to interpret gradations of color. For example, a zone with a yellow background that accumulates blue latex particles will appear yellow if no particles are bound, green if low levels of particles are bound and blue if high levels are bound.

### Wash (Surfactant Treatments, Architectures, Channels to Absorbent Pad)

The device may contain a wash layer. In preferred embodiments, the wash layer takes the form of one or more patterned channels within a porous substrate. Porous substrates for the wash layer can be selected from, but limited to: glass fiber, chromatography paper, nitrocellulose, non-woven polyester, or nylon. In some embodiments, “super absorbent” polymers such as poly(acrylic acid), poly(acrylonitrile), poly(acrylamide) or other hydrogels may be incorporated into the wash layer. Natural absorbent materials such as chamois leather may also be incorporated as part of the wash layer. The channels act to wick non-bound material (often colored material such as latex particles or colloidal gold) away from the capture zone. In some instances, a channel provides an ideal means of focusing particles in a stream away from the capture zone which serves to decrease background signal. The length of the channel ultimately controls the total volume of fluid that will pass through the device. The channel may be straight or have one or more turns or splits present. In some embodiments, the wash channel may take the form of a serpentine path. In some embodiments the wash channel may connect to an absorbent pad located in the same plane or underneath the wash channel. In some embodiments, the channels or absorbent layers are treated with surfactants, polymers, or other agents to increase surface energy and wicking rates.

### Multiplex—Port & Split Layers (Architectures, Possible for Thousands of Zones)

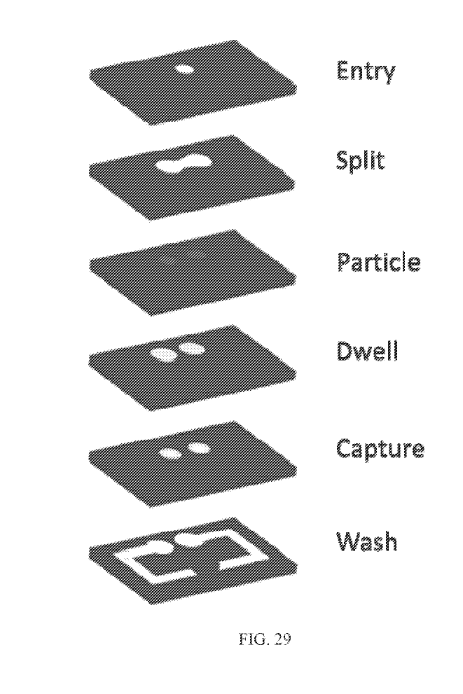

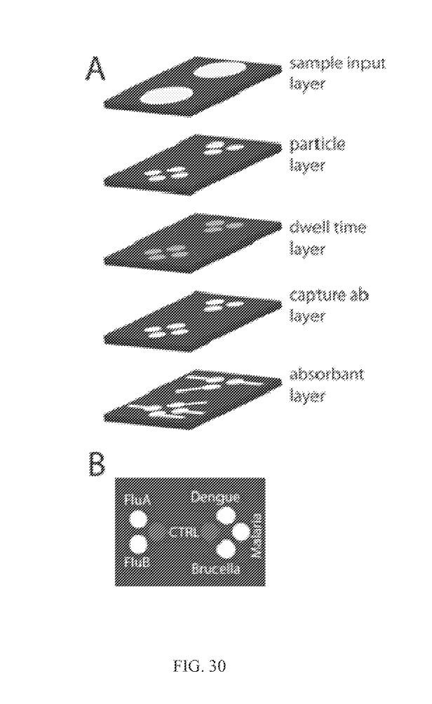

Additional layers may be provided which allow for a small volume of sample to be placed into the device and automatically split multiple times such that multiple assays can be run in parallel. An example of such a device, designed to run two immunoassay tests in parallel is illustrated in FIG. 29. By using similar design principles, many tests can be run in parallel. FIGS. 30 & 31 illustrate multi-layer layer designs. It is possible in theory for hundreds of immunoassays or other assay types to be run in parallel in this fashion. Whitesides et al. describe a 3-D device capable of splitting a small volume of fluid into hundreds of zones in (PNAS, 2008, 105, 19606) and in WO 2009/121037. In some embodiments, these “split layers” may perform selective treatments on part of a sample in preparation for a given assay. For example, buffer salts could be deposited into a zone or channel which could alter the pH or ionic strength of the solution selectively for an assay.

In some embodiments, the split layers are not necessary and a filter membrane that spans the area of the multiple reagent layers can be used. A notional example of such a device is shown in FIG. 30.

### Methods for Fabrication (Wax Printing, Screen Printing, Lamination, Etc.)

The manufacturing unit operations for Immunoassay fabrication can be broken up into approximately 6 steps.

i.) Patterning of the paper substrate with hydrophobic barriers

ii.) Patterning of adhesive by screen printing

iii.) Deposition of biological/chemical reagents

iv.) Layer alignment and assembly

v.) Attachment of Plasma separation membrane

vi.) Lamination and packaging

Features of the manufacturing process used to prepare the above devices are described below:

### I. Patterning of Paper

### Wax Printing

A preferred method for patterning paper to be used in the immunoassay is wax printing. The method is described in detail by Whitesides et al. in Anal Chem 81:7091-7095, and WO 2010/102294. The device is designed on a computer and the hydrophobic walls of the microfluidic channels are printed onto a letter-sized sheet of paper using a commercial printer with solid-ink technology (Xerox Phaser). The printer melts the wax-based solid ink and deposits it on top of the paper. The sheet is then heated to above the melting point of the wax, which permeates through the paper, creating a hydrophobic barrier through the entire thickness of the paper. Some spreading occurs during the heating step, but it is reproducible based on the paper and thickness of the printed line and can be incorporated into the design. Channels patterned in the paper wick microliter volumes of fluids by capillary action and distribute the fluids into test zones where independent assays take place.

### Alternative Methods for Patterning Paper

Other method embodiments may use paper soaked in photoresist which is then exposed to UV light through a photomask with a desired pattern. The unexposed regions are then washed away with a suitable solvent, leaving behind crosslinked hydrophobic regions that penetrate the thickness of the paper. Feature sizes as small as 100 μm have been demonstrated using this technique. Examples of this method of patterning can be found in prior work from the Whitesides lab in Angew. Chem. Int. Ed. 2007, 46, 1318-1320 and WO 2008/049083. In further embodiments, there is a host of other large-scale printing and patterning techniques that can be used to deposit hydrophobic barriers into paper to meet the requirements of the immunoassay. These methods include, but are not limited to: screen-printing, gravure printing, contact printing, flexographic printing, hot embossing, ink jet printing, and batik printing.

### II. Patterning of Adhesive by Screen Printing

In several embodiments, it is necessary to adhere the layers together in such a way that fluids can wick in the z-direction to entry points in the next layer of paper. One method of accomplishing this is by using double-sided adhesive tape with holes cut into the desired pattern through which fluid can flow. This method has been described in previous work from the Whitesides lab (PNAS, 2008, 105, 19606). This particular method requires that a hydrophilic powder (e.g., cellulose) is added in the cut aperture between the layers of paper formed by the thickness of the tape. This and the added cost of tape to the device make this method non-ideal for large-scale fabrication. A preferred method for assembly of 3-D devices is to use simple and scalable screen-printing techniques to deposit very thin layers of adhesive onto paper in the desired pattern. In this manner, a hydrophobic, pressure-sensitive adhesive (Unitak 131 sold by Henkel Corporation) can be applied to the paper. Once adhesive is applied, pre-made sheets can be stored by laminating the adhesive side to a non-adhesive release layer, commonly seen in other adhesive products such as labels and tapes. In further embodiments, a stencil can be fabricated and pressed against a sheet of patterned paper in such a way that certain features are covered. An adhesive may then be deposited from an aerosol spray onto the remaining exposed regions.

In some embodiments, a weak adhesive bond may be desired to facilitate peeling between certain layers. To accomplish this, a low-tack adhesive may be used that allows for sealing to occur as fluids are wicking through the device channels, but is also easily separated to view the capture layer. In some embodiments, the screen printed pattern of adhesive can allow a corner of selected device layers to be free of adhesive, therefore enabling easy peeling where desired.

### III. Deposition of Reagents

In preferred embodiments, it is necessary to deposit chemical and/or biological assay reagents into regions of the device. The reagents react with analytes present in a bodily fluid and which yields a response (i.e., colorimetric or electrochemical) that can indicate the concentration of a particular analyte. It is often necessary to formulate reagents with appropriate stabilizers (e.g., sugars) to preserve function once dried. In one embodiment, useful for prototyping and small scale production (e.g., 100's of devices per day), deposition of reagents is done by hand using micropipettes and repeat pipetters. A typical volume deposited is between 0.5 and 5 μL. In preferred embodiments for larger scale production, precision liquid deposition machines can be used. Two examples of such tools are the AD3400 available from BioDot, Inc. and the Diamatix DMP-2800 Ink Jet printer available from Fujifilm. Both of these units are able to rapidly dispense precise volumes (contact-free) of fluid down to nL volumes in a programmed pattern. Additionally, such units can be adapted to continuous manufacturing lines for large scale production.

### IV. Layer Alignment and Assembly

In preferred methods of manufacture, devices are assembled in full sheets. For this to occur, it is imperative that patterned regions precisely align to make the necessary fluidic junctions possible between layers. A simple and scalable way to accomplish this is to cut precise holes in the paper layers such that the sheets can slide onto peg boards. Each layer can then be applied to the peg board such that features are rapidly aligned correctly. The adhesive applied earlier acts to lock the sheets in place once in contact. In continuous manufacturing, a similar method can be used on reels containing pegs such as that used in Dot-Matrix Printing. Alternatively, laser web guides can be used to precisely align sheets before lamination.

### V. Attachment of Plasma Separation Membrane

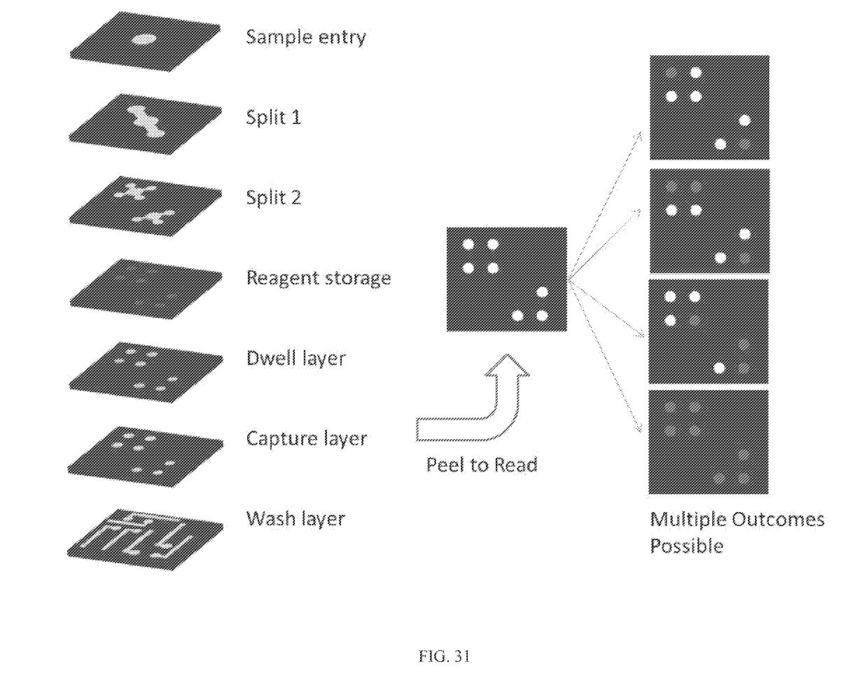

In some embodiments, a plasma separation membrane (Pall Corporation) is cut and placed at the entry point of the device. The membrane serves as a reservoir to collect a blood drop and importantly to filter red cells out of the blood and allow plasma to wick into the device. Accordingly, embodiments of the present invention utilize a “pick and place” method consisting of the following steps (illustrated in FIG. 8):
i) A sheet of Pall membrane is cut into densely packed circles 1 cm in diameter using a laser cutter or die cutter. The cut sheet is laminated to surface with low adhesion such as a low-tack laminate sheet, or a rubbery sheet. In preferred embodiments, the cut membrane sheet is adhered to a PET film coated with PDMS.
ii) A sheet of adhesive laminate is cut using a knife plotter, laser cutter, or die cutter such that it contains apertures which act as an entry point into the filter/device (top layer of FIG. 31). The holes in the laminate sheet are 0.75 cm in diameter.
iii) A non-adhesive masking layer is cut from waxy cardstock, or other materials with low adhesion, in a pattern to have holes that are 1.13 cm in diameter, slightly larger than the filters.
iv) The previously cut laminate containing 0.75 cm holes and the masking layer are adhered together such that the laminate aperture is in the middle of the blocking layer aperture.
v) The stack is placed over the densely cut membrane sheet in such a way as to only pick up filter membrane discs that align with the cut laminate sheet. The others are blocked by the masking layer.
vi) The stack is laminated and the adhesive laminate layer containing 0.75 cm holes is peeled away which, as it is peeled, adheres a filter over each laminate aperture on the laminate sheet while leaving the others behind for the next set of devices.
vii) The laminate layer, now with a filter membrane adhered under each aperture, is adhered to a stack of multiple layers of patterned paper which are adhered together by screen printed adhesive.

In this way, the maximum area of the membrane material can be converted into useable filtration discs for devices. Using dye-cutting techniques and simple laminators, this process can be easily automated into large scale-production.

### VI. Lamination and Packaging

After the steps above have taken place, the final step in device manufacturing is to laminate the stack of patterned paper (and filters, etc., if required). For this step, a “cold lamination” sheet consisting of a PET film with adhesive on one side is used. The film protects the devices and provides the outer hydrophobic layer for the patterned zones. Devices are then cut, placed in foil-lined bags containing desiccant and heat sealed.

### Sample-Handling

In some embodiments, it is useful to have certain sample handling features built into the device itself. One such feature is a simple plastic cover that protects the sample entry aperture. It is potentially desired that after a drop of blood is introduced to the device via the entry aperture and into the filter membrane that a plastic cover then seals the aperture to slow the evaporation and drying of the fluids in the device.

In further embodiments, it may be desired to have a built-in capillary capable of drawing a precise volume of blood into the device by simply making contact with the droplet. This minimizes user operations and ensures reproducibility in the volume of sample introduced to the device.

In still further embodiments, a device may be envisioned that contains a built-in lancet which is disposed of along with the device after use.

In some embodiments, the device is used as part of a kit containing a glass or plastic capillary tube; in preferred embodiments the tube is plastic, such as the MicroSafte Tube available from Safe-Tec®. In some embodiments, the kit contains a lancet; in preferred embodiments, the lancet is a spring-loaded lancet, such as those available from Surgilance™. In still further embodiments, a kit will contain patterned paper devices, a lancet, a capillary tube, a bandage, an alcohol swab, latex gloves, and a colorimetric read guide for interpretation of results.

Various Filters—Glass Fiber, Paper with Anti-RBC Antibodies, Etc.

A potential component of the present invention is a filter pad which is incorporated into the device and serves as a filter for blood cells (as well as dirt, fibers, etc.) for the isolation of plasma which then wicks into the device. In preferred embodiments, the filter is a Vivd™ membrane available from Pall corporation. In other embodiments, the membrane can be a glass fiber membrane, or even a paper filter. In other embodiments, anti-blood cell antibodies may be attached to the membrane to facilitate capture of cells. In further embodiments, “scrubbing agents” may be added to the filter membrane or paper channels that are capable of capturing substances that may interfere with the reaction chemistry.

### Materials

Nearly any porous material can be patterned by the methods disclosed. Accordingly, many materials can be patterned to generate an immunoassay according to the present invention. Materials include, but are not limited to: paper, chromatography paper, nitrocellulose, non-woven polymeric materials, lab wipes, nylon membranes such as Immunodyne® membranes sold by Pall® corporation. A preferred material is Whatman® no. 1 chromatography paper.

### Storage and Stability—Use of Stabilizing Agents for Enzymes

In some embodiments, stabilizers are added to the reagent zones to further stabilize the antibodies spotted onto the paper. In further embodiments the stabilizers include but are not limited to: trehalose, poly(ethylene glycol), poly(vinyl alcohol), poly(vinyl pyrrolidone), gelatin, dextran, mannose, sucrose, glucose, albumin, poly(ethylene imine), silk, and arabinogalactan.

In preferred embodiments, the stabilizers are sugars. A particularly useful method for stabilizing antibodies and other proteins, vacuum foam drying, is described by Bronshtein et al. in US. Pat. No. 6,509,146 and is incorporated herein by reference.

### Timers

A timer may be incorporated into the device which serves to indicate to an operator when the device should be read. Such timers have been described by Phillips et al. in Anal. Chem., 2010, 82, 8071-8078 which is incorporated herein by reference.

In further embodiments, a timer takes the form of a multi-layer device containing a channel of defined length and width such that fluid takes a predictable amount of time to travel to the end of the channel. Upon addition of sample to the device, fluid immediately begins to wick down the defined paper channels. As the fluid wets the channel, it can reveal printed messages on the reverse side of the paper as the paper becomes wet, and therefore transparent. This concept is illustrated in FIG. 8.

In some embodiments, the positive control can act as a timer for the test in that when the positive control is fully developed, the device can be read. In further embodiments, the assay may be sensitive to heat or humidity leading to an acceleration or deceleration of the assay. In this situation, a positive control can be tailored such that it exhibits the same acceleration or deceleration effect. In this way, the device is still read when the positive control is developed, and no timer is needed.

### “Plus and Minus” Readout

In some embodiments, visual results can take the form of a “plus” sign “+” or “minus” sign “−”. This is accomplished by having a horizontal control line crossed by a vertical sample line. Lines can be generated by printing capture antibodies using plotters, inkjet printers, etc. In this way, a sample which is negative for a particular analyte will only activate the control line and develop as negative minus “−” symbol while a sample which contains a particular analyte will develop both the horizontal and vertical lines and reveal a plus “+” symbol.

### ELISA Concepts

In some embodiments, enzyme-linked immune-sorbent assays (ELISA) can be run on multilayer devices generated from patterned paper.

### Multiple Wash Design

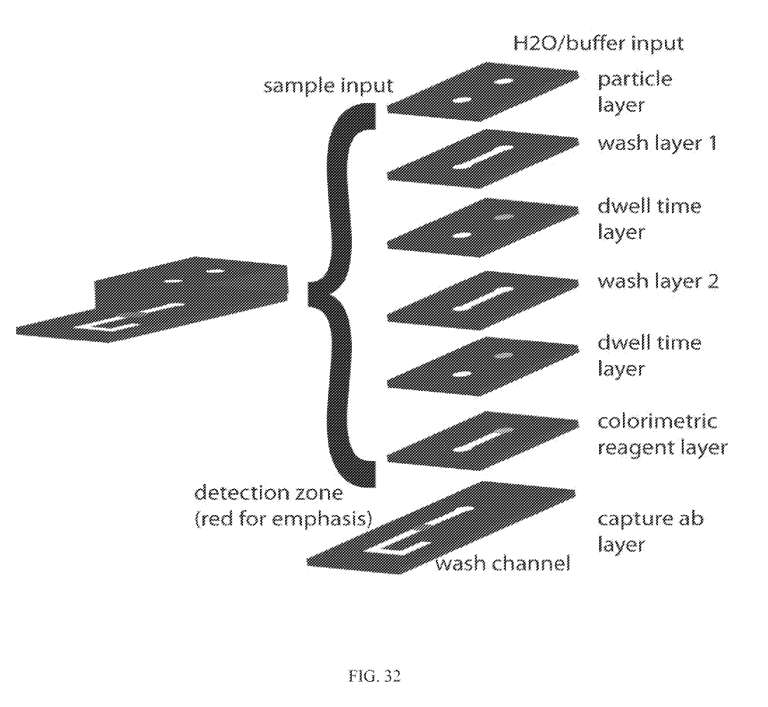

In one design, a multi-layer 3 dimensional device is constructed such that autonomous washing can take place after sample addition. An example of one such design is shown in FIG. 32. In this design, an Enzyme-Antibody conjugate is stored dry on the top layer where it can bind to an antigen present in a sample. The partial immune complex then rapidly migrates to the capture antibody spot where it is bound and residual material is migrated away via the wash channel. At the same time, a drop of buffer is added to a separate port where it is delayed by multiple dwell zones. Eventually (after the residual conjugate has migrated down the wash channel) the buffer picks up a colorimetric developer such as TMB (3,3′,5,5′-Tetramethylbenzidine). The developer migrates to the bound Enzyme-conjugate to produce a color in the read zone. If no antigen is present, no color will be generated in this zone. Variations on this concept will be obvious to those skilled in the art and are hereby incorporated.

### “No Wash” ELISA Concepts

In other designs devices similar to those described in U.S. Pat. Nos. 4,446,232 and 4,587,102 can be adopted to the patterned paper device platform. Improvements to this concept described herein are: i) the use of patterned zones and channels to dramatically reduce the amount of sample necessary to conduct these assays and ii) the addition of split channels in a 3-D formal which would allow for multiple assays to be conducted simultaneously.

Preparation of Exemplary Device and Analysis of hCG-LOD, Sensitivity, Specificity, Repeatability

A. Device Fabrication

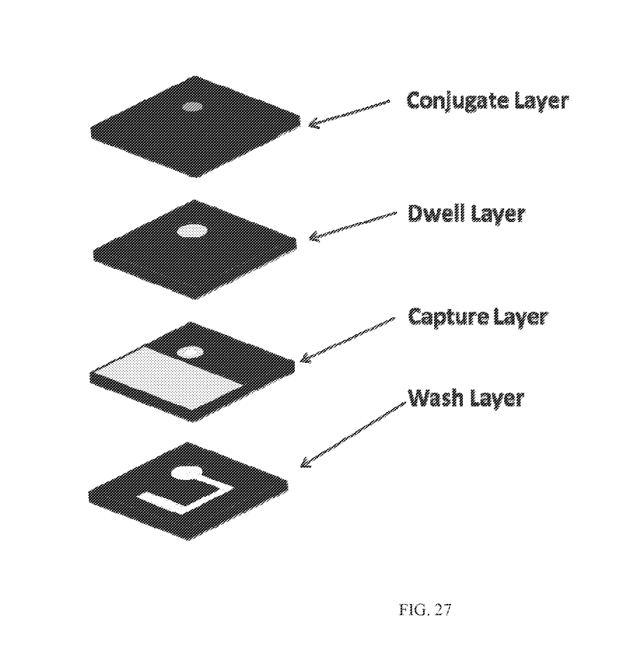

A reagent layer, dwell layer, and wash layer were printed onto 8.5×11″ sheets of Whatman no. 1 chromatography paper according to the design shown in FIG. 27 using a Xerox Phaser 8560 DN wax printer. Additionally, a capture layer was printed onto nitrocellulose membrane by taping nitrocellulose membrane to a sheet of copy paper and feeding it into the printer. All layers were heated at 150° C. for 45 seconds to ensure that the wax migrated through the thickness of the porous sheets. The capture layer zones were spotted with 2 μL of a solution containing 0.1% Zwittergent and dried in an oven at 70° C. for ten minutes. Following this, 2 μL of a solution containing 1 mg/mL of hCG capture antibody (Sekisui Diagnostics) was spotted onto the zones and dried at 70° C. for 10 minutes. Alignment marks were punched in each layer using a hole puncher. To each zone in the reagent layer, 2 μL of a 1.2M solution of trehalose in water was applied and the spots were allowed to dry for 5 min in an oven at 70° C. Following this, 2 μL of a 0.07% suspension of blue polystyrene latex beads coated with hCG antibody (Sekisui Diagnostics) was applied to each zone and allowed to dry for 5 min in an oven at 70° C.

A pressure-sensitive adhesive (UNITAK 131, Henkel) was applied to the top of each layer by screen printing. The printing screen was patterned using known methods with photocurable emulsion (Atlas Screen Printing Supply) such that the active zones of the device layers did not receive adhesive but the remaining areas did. The layers were dried using a heat gun for two minutes to drive off water from the adhesive leaving behind a patterned, tacky layer of adhesive with “holes” over the zones. This screen-printing process was repeated for each layer. The layers were then aligned to a peg board using the previously punched alignment holes.

B. Buffer Testing

To each device, 30 μL of a buffered solution containing either high (500 mIUs/mL), low (50 mIUs/mL), or negative (0 mIUs/mL) of hCG (Sekisui Diagnostics) were added to the entry zone of the reagent layer using a micropipette. The drop was allowed wick into the device for 10 minutes and the device was peeled apart to reveal the capture layer. The results were recorded using a desktop scanner (Canon). A dark blue spot was observed for the sample containing high levels of hCG, a lighter blue spot was observed for the sample containing low levels of hCG and a white-gray spot was observed for the sample containing zero hCG.

C. Limit of Detection

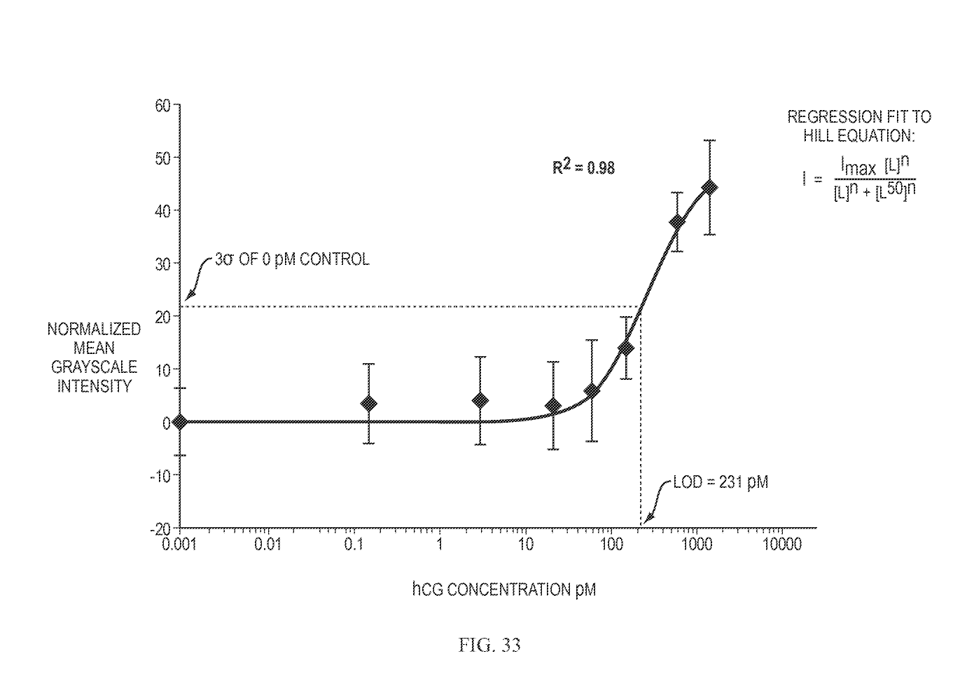

A Limit of detection (LOD) curve was generated for the hCG assay using standard statistical methods. Color intensity was quantified in each zone by using a desktop scanner to digitize the image and analysis software (ImageJ) to obtain a value. A calibration plot of the output signal versus the concentration of hCG in the buffer sample (N=7 for each concentration) is shown in FIG. 33. A total of 8 different concentrations were measured. The solid line represents a non-linear regression of Hill Equation: I=Iₘₐₓ[L]ⁿ/([L]ⁿ+[L₅₀]ⁿ), where Iₘₐₓ=48.36, [L₅₀]=257.32 pM, n=1.413, and R²=0.98. The error bars represent one standard deviation (σ). The calculated LOD was 231 pM for the hCGassay. A qualitative assessment of the limit of detection—defined as the lowest concentration that 3 operators could distinguish from a negative control—was determined to by 10 mIU/mL.

D. Sensitivity, Specificity, and Repeatability

Sensitivity and specificity were measured by testing 10 clinical urine specimens containing hCG and 10 negative specimens. The device correctly identified 10/10 positive and 10/10 negative samples (Table 1). Repeatability was measured by calculating the coefficient of variation for multiple concentrations of hCG. Within-run (all devices from the same lot) and between run (devices tested across a 10 day span) precision were determined CV's were less than 10% for all conditions.

TABLE 1
Visual limit of detection = 10-20 mIUs
Sensitivity - 10/10 positive clinical urine specimens correctly identified
Specificity - 10/10 negative clinical urine specimens correctly identified
Within-run precision (22 tests)

| | |
|---|---|
| Low hCG | CV = 4.7% |
| NeghCG | CV = 3.4% |

Between-run precision (30 tests)

| | |
|---|---|
| High hCG | CV = 8.2% |
| Low hCG | CV = 4.2% |
| NeghCG | CV = 4.4% |

### Preparation of Exemplary Device Using a hCG Colloidal Gold Conjugate

A device was constructed similarly to the above described in the paragraphs above, only with the following changes:
Immunodyne ABC membrane (0.45 μm) available from Pall corporation was used in place of nitrocellulose. The membrane was patterned as before and spots were blocked with a casein buffer.
2 μL of a 1 mg/mL hCG colloidal gold conjugate suspension (AbCam) was used in place of the blue polystyrene latex beads.
A different vendor was used for the capture antibody (AbCam) which was spotted at 1 mg/mL.
   Buffer samples (1% bovine serum albumin in Tris) were prepared to contain high (250 mIU/mL), low (50 mIU/mL), and control (0 hCG) amounts of hCG. 30 μL samples were added to the devices as described in the paragraphs above. Results show very strong signal from the high and low samples and very low background from the control. The data show that the platform can be used for both latex and colloidal gold systems.
   Preparation of hCG-C Difficile Toxin A Multiplex

A. Device Fabrication

A port layer, split layer, reagent layer, dwell layer, and wash layer were printed onto 8.5×11″ sheets of Whatman no. 1 chromatography paper according to the design shown in FIG. 29 using a Xerox Phaser 8560 DN wax printer. Additionally, a capture layer was printed onto nitrocellulose membrane by taping nitrocellulose membrane to a sheet of copy paper and feeding it into the printer. All layers were heated at 150° C. for 45 seconds to ensure that the wax migrated through the thickness of the porous sheets. The capture layer zones were spotted with 2 μL of a solution containing 0.1% Zwittergent and dried in an oven at 70° C. for ten minutes. Following this, 2 μL of a solution containing 1 mg/mL of hCG capture antibody (Sekisui Diagnostics) was spotted onto the left zones and dried at 70° C. for 10 minutes. Additionally, 2 μL of a 3.36 mg/mL solution of capture antibody for c. difficile toxin A were spotted and dried at 70° C. for ten minutes. Alignment marks were punched in each layer using a hole puncher. To each zone in the reagent layer, 2 μL of a 1.2M solution of trehalose in water was applied and the spots were allowed to dry for 5 min in an oven at 70° C. Following this, 2 μL of a 0.4% suspension of blue polystyrene latex beads coated with hCG antibody (Sekisui Diagnostics) was applied to the left zone and allowed to dry for 5 min in an oven at 70° C. Additionally, 2 μL of a 0.4% suspension of blue polystyrene latex beads coated with c. difficile toxin A antibody (Sekisui Diagnostics) was applied to the right zone and allowed to dry for 5 minutes in an oven at 70° C.

A pressure-sensitive adhesive (UNITAK 131, Henkel) was applied to the top of each layer by screen printing. The printing screen was patterned using known methods with photocurable emulsion (Atlas Screen Printing Supply) such that the active zones of the device layers did not receive adhesive but the remaining areas did. The layers were dried using a heat gun for two minutes to drive off water from the adhesive leaving behind a patterned, tacky layer of adhesive with “holes” over the zones. This screen-printing process was repeated for each layer. The layers were then aligned to a peg board using the previously punched alignment holes.

Tests were performed by applying initially 30 μL of sample followed by 2×30 μL of the sample as a chase buffer. Four separate solutions were made: +/+ solution containing 400 ng/mL of C-Diff toxin A and 500 mIU/mL of hCG, +/− solution containing 500 mIU/mL of hCG, −/+ solution containing 400 ng/mL of C-Diff toxin A, and −/− containing only buffer. Tests designed in this manner were able to distinguish between samples containing either c. difficile, hCG, neither or both. These results exhibit the utility of this format for multiplexed immunoassay diagnostics.

Preparation of a Multiplex with Clinical Chemistry (should Multiplex AST and ALT with hCG as Example).

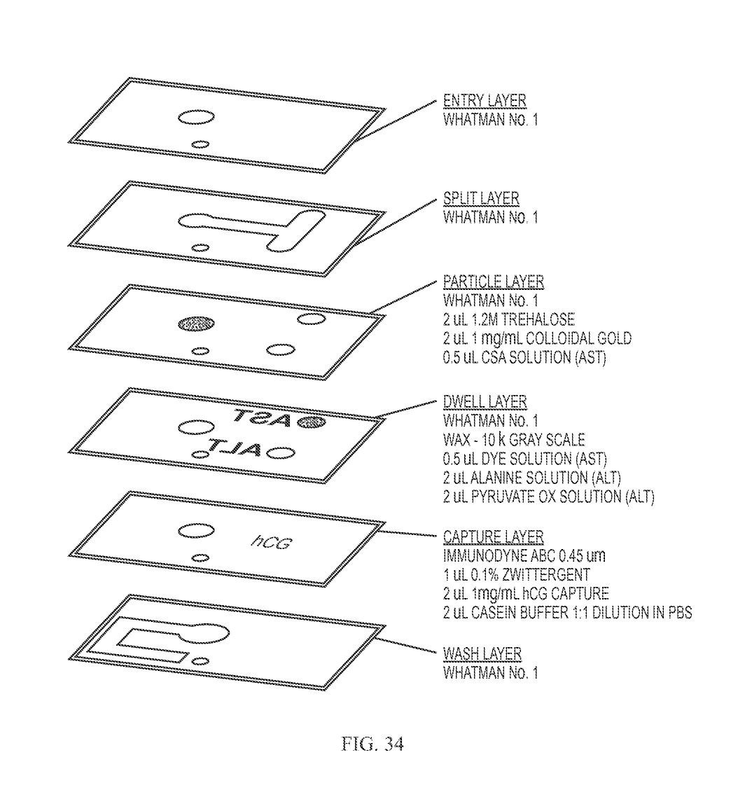

A port layer, split layer, reagent layer, dwell layer, and wash layer were printed onto 8.5×11″ sheets of Whatman no. 1 chromatography paper according to the design shown in FIG. 34 using a Xerox Phaser 8560 DN wax printer. Additionally, a capture layer was printed onto Immunodyne ABC membrane (Pall) by taping nitrocellulose membrane to a sheet of copy paper and feeding it into the printer. All layers were heated at 150° C. for 45 seconds to ensure that the wax migrated through the thickness of the porous sheets. The capture layer zones were spotted with 2 μL of a solution containing 0.1% Zwittergent and dried in an oven at 70° C. for ten minutes. Following this, 2 μL of a solution containing 1 mg/mL of hCG capture antibody (AbCam) was spotted onto the left zones and dried at 70° C. for 10 minutes. Finally, 2 μL of casein blocking buffer were spotted and dried at 70° C. for 10 minutes. Alignment marks were punched in each layer using a hole puncher. 2 μL of a 1.2M solution of trehalose in water was applied to the hCG spot and allowed to dry for 5 min in an oven at 70° C. Following this, 2 μL of a 1 mg/mL suspension of colloidal gold coated with hCG antibody (AbCam) was applied to the left zone and allowed to dry for 5 min in an oven at 70° C.

Additionally, 2 μL of a solution containing 1M L-alanine (Sigma Aldrich), 30 mM α-ketoglutaric acid (Sigma Aldrich), 2 mM KH₂PO₄ (Sigma Aldrich), 20 mM MgCl₂ (Sigma Aldrich), 2 mM Thiamine Pyrophosphate (MP Biosciences), 2 mM of 4-aminoantipyrine (Sigma Aldrich) and 25 U/mL (0.1 mg/mL) Horseradish Peroxidase (HRP) (Sigma Aldrich) in 200 mMTris buffer (pH=7.4) was spotted into the ALT spot on the dwell layer, allowed to dry at room temperature, followed by 2 μL of a solution containing 5 wt % PEG (MW=35,000), 5 mM dimethylaminobenzoic acid (DABA), and 100 U/L of pyruvate oxidase.

For the AST assay, 0.5 μL of a solution containing 17 wt % of cysteine sulfonic acid, 1.4% alpha-ketoglutaric acid, and 2 mM EDTA in PBS was spotted onto the AST zone in the particle layer and 0.5 μL of a solution containing 0.6% Methyl Green and 0.05% Rhodamine B in 1% PVA was spotted onto the AST zone in the dwell layer. The spots were allowed to dry at room temperature.

A pressure-sensitive adhesive (UNITAK 131, Henkel) was applied to the top of each layer by screen printing. The printing screen was patterned using known methods with photocurable emulsion (Atlas Screen Printing Supply) such that the active zones of the device layers did not receive adhesive but the remaining areas did. The layers were dried using a heat gun for 2 minutes to drive off water from the adhesive leaving behind a patterned, tacky layer of adhesive with “holes” over the zones. This screen-printing process was repeated for each layer. The layers were then aligned to a peg board using the previously punched alignment holes. Additionally a small piece of adhesive lamination (Fellowes) was applied underneath the dwell layer on the ALT/AST side.

Tests were performed by applying 40 μL of sample. Positive samples contained 250 mIU hCG, 2500 U/L ALT, and 2500 U/L AST in 3.5% BSA/artificial blood plasma. Negative samples contained only 3.5% BSA/artificial blood plasma. After sample addition the device was set aside for 15 minutes and then peeled open between the Dwell and Capture layers to reveal the results. The assays were functional when combined into a single device run from a single sample.

### EXAMPLES

### Example 1

### Surface Fluorescence Measurement for LAMP Quantification in Paper Materials

Detection hardware: Fiber optic probe spectrofluorometer system (Ocean Optics): USB4000 portable spectrofluorometer, pulsed xenon lamp light source, fiber optic cable, reflection probe, linear variable filter holder equipped with filter, patch cable, sample and probe holder. Detection reagent: Propidium iodide (0.1 mg/mL)

### Methods

We investigated using fluorescence emission from a DNA intercalating agent (PI) as a means of measuring nucleotide amplification conducted in the test zone of our device. To quantify the extent of LAMP amplification in the paper disc reactors that comprise the test zone of our device, a fiber optic probe spectrofluorometer system (FIG. 4) was procured and optimized for the excitation of PI and measurement of the resulting fluorescence emission of the intercalated dye. The system contained a spectrofluorometer, a pulsed xenon lamp light source, and a specially designed reflection probe. The excitation leg of a fiber optic cable is connected to the xenon lamp via a linear variable filter holder and patch cable, allowing the shaping of the excitation spectrum within a 25 nm band-pass. Filtered light is delivered via the excitation cable to the probe holder containing the sample. Simultaneously, fluorescence emitted within the probe holder in response to the excitatory light is collected by the fluorescence probe. The emission leg of the fiber optic cable, which connects the fluorescence probe and the spectrofluorometer, relays fluorescence emission data collected by the probe to the spectrofluorometer. The final emission spectra is a combination of the specific emission of the PI intercalated and/or free in solution and any reflected light from the surface.

We optimized the following parameters with regard to the above system: (i) height of the probe over the surface of the sample, (ii) integration time and sample averaging to control the xenon lamp emission flash rate, (iii) electronic filters to eliminate system noise, (iv) dye concentration, and (v) measurement protocols. The parameters of the system related to spectrofluorometer performance, such as flash rates and electronic filters, were adjusted to maximize excitation peak intensity and stability. Optimization resulted in minimal noise and less fluctuations in excitation peak intensity. Optimal bandpass cut-off wavelength was achieved using a white reflection standard. Optimal probe height was adjusted to maximize the surface area of the reaction disc exposed to the excitation light while maintaining a short distance for the collection of the emission of the intercalated dye. This goal was achieved via utilization of plastic spacer layers inserted under the probe holder. Dye concentration was optimized to avoid spectral changes resulting from colorimetric shifts at high concentrations while maintaining strong fluorescence emission above the background. Ultimately, 0.1 mg/mL PI and a single spacer layer proved optimal. Finally, spectral data for LAMP positive and negative controls were analyzed to determine the appropriate wavelength or parametric analysis (e.g., excitation intensity divided by emission peak intensity) yielding a simple and consistent method to discriminate the signals produced by the two dye states. The emission peak intensity, recorded at 616.95 nm, provided the best signal discrimination. To avoid variability in signal obtained following addition of PI solution to the disc, we incorporated steps of unsealing the reactor post-incubation, drying at 65° C. for 5 minutes, and rehydrating the disc in 4 μL of PI. The PI solution was allowed to diffuse through the disc for five minutes before readings were taken.

### Example 2

### Analytical Sensitivity of LAMP Reactions in Paper

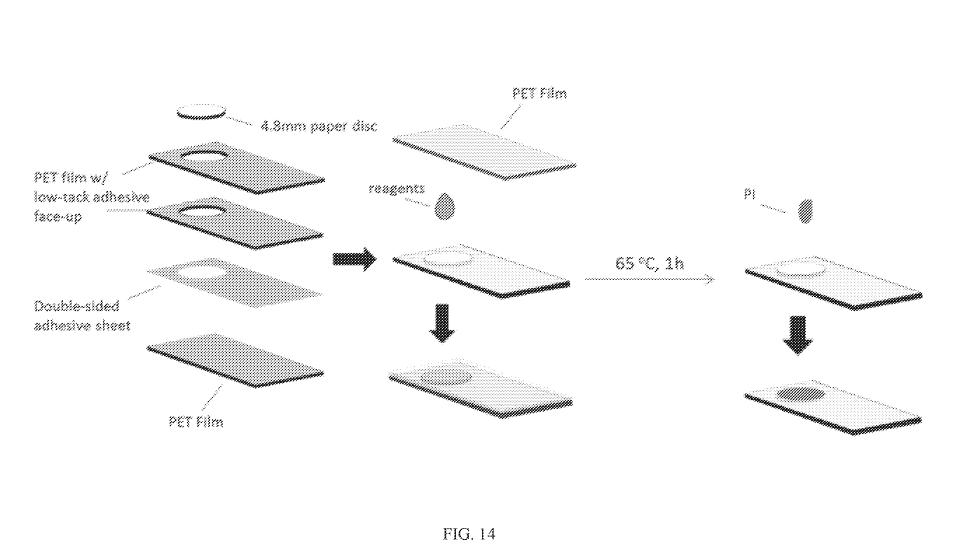

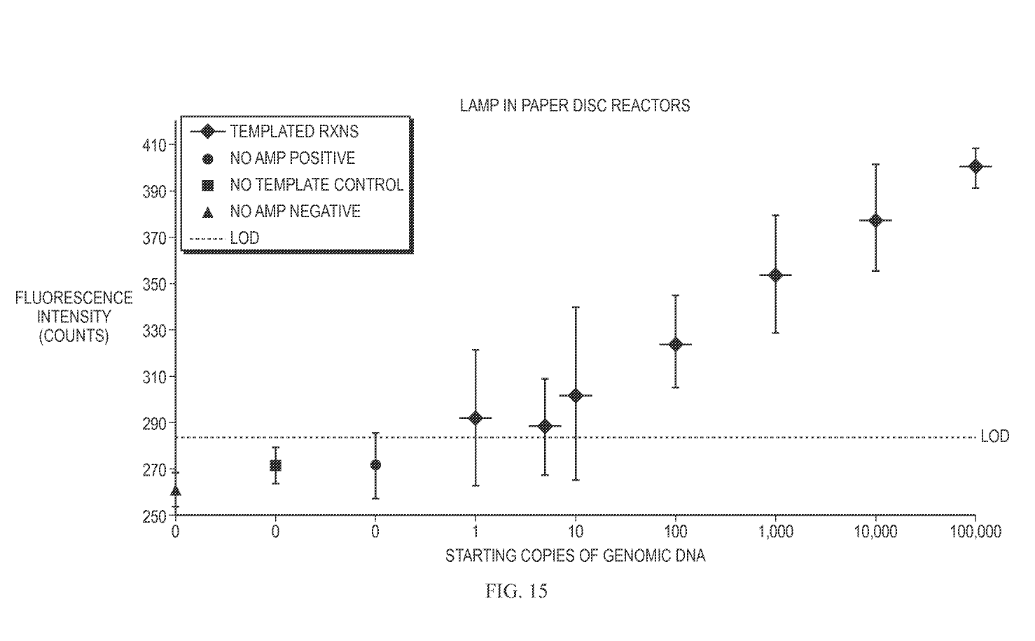

Experiments were performed to the determine the sensitivity and signal variability obtained from fluorescent reading of LAMP reactions in paper detected via PI addition. To measure sensitivity of the detection system, various starting concentrations of template DNA were prepared by serial dilution. Reactions were mixed in 200 mL microtubes, and 4 mL aliquots were deposited onto each disc housed within a PET reactor (FIG. 14), allowing for multiple replicates from each tube. The PET reactors were constructed such that a 4.8 mm paper disc was placed within a hole cut out of a stack of three consecutive layers of PET film, a low-tack adhesive strip, and a double-sided adhesive strip. This assembly was seated on top of a layer of PET film (FIG. 14). The reactors were sealed following addition of the reaction mixture using another layer of PET film. The reactors were then incubated at 65° C. for 1 hour in an oven, and processed for fluorescence measurements as in Example 1. Seven replicates were performed for each template concentration. Reaction discs containing 100 starting copies of the E. coli genome consistently amplified, producing emission peak intensities well above the limit of detection defined as three times the standard deviation of a negative control containing no polymerase (FIG. 15).

Samples containing 100,000 starting copies and an inactive polymerase showed fluorescence intensities within the error of the negative control indicating that the starting concentration of template DNA, within the range used, does not produce interfering fluorescence due to PI intercalation. This result, as well as results for paper-based reactions at selected concentrations around the determined limit of detection, were confirmed with agarose gel electrophoresis (not shown).

### Example 3

### Evaluation of Sliding Strip Device Materials

Experiments were performed to determine the effects of potential materials to be used in the construction of the sliding strip device on which the LAMP reaction takes place. For the sliding strip device to function, the strip containing the disc must be capable of remaining sealed while still providing aqueous fluidic contact. An evaporation-resistant seal can be maintained during movement with the use of grease. To determine optimal materials that achieve these goals, stationary reactors were constructed out of test materials including Fellowes brand PET laminate, Flexmark 400PM PET film with low-tack adhesive, Flexmount double-sided adhesive sheets, 3M PET transparency film, Corning Sylgard silicone grease, and Krytox fluorinated polymer grease.

For ease of fabrication, it is desirable to have an adhesive substrate for the base of the strip. Thus, stationary reactors were constructed where the reactor is in contact with only raw PET on the top and bottom faces, and negligible contact with low-tack or doublesided adhesives as a control. Additional constructs were constructed where the bottom of the reactor is replaced with Fellowes laminate with adhesive contacting the disc, Flexmark 400PM with lowtack adhesive contacting the disc, or Flexmount double-sided adhesive contacting the disc. Further, reactors were constructed where the top of the reactor was sealed with silicone grease screen-printed onto a raw PET lid, Krytox grease screen-printed onto a raw PET lid, and Krytox grease screen-printed onto laminate affixed to wax-patterned paper. All reactors were constructed using the design shown in FIG. 14.

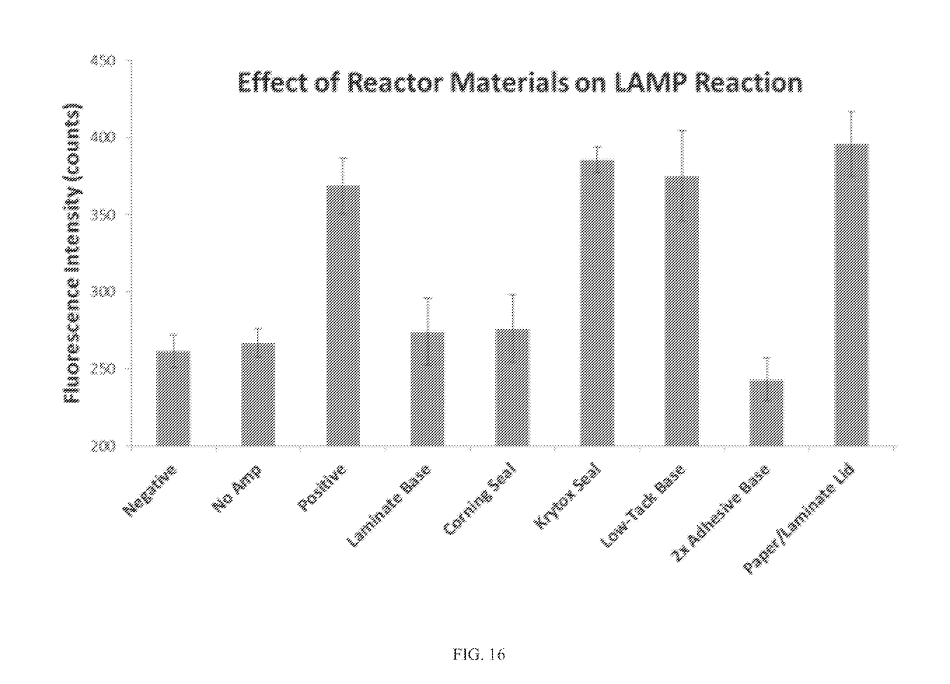

LAMP reactions were conducted as described in Example 1 using a starting template concentration of 10,000 E. coli genome copies for each condition. Replicates of three were tested for each configuration. Both the Fellowes laminate base (FIG. 16, Laminate Base) and the silicone grease (FIG. 16, Corning Seal) inhibited the LAMP reaction, while the low-tack laminate (FIG. 16, Low-Tack Base) and Krytox grease (FIG. 16, Krytox Seal) did not do so (FIG. 16). The double-sided adhesive was mechanically not useful as the discs adhered too greatly and could not easily be removed for fluorescence measurement, resulting in a loss of ˜25% of the paper thickness.

### Example 4

### LAMP in Sliding Strip Device

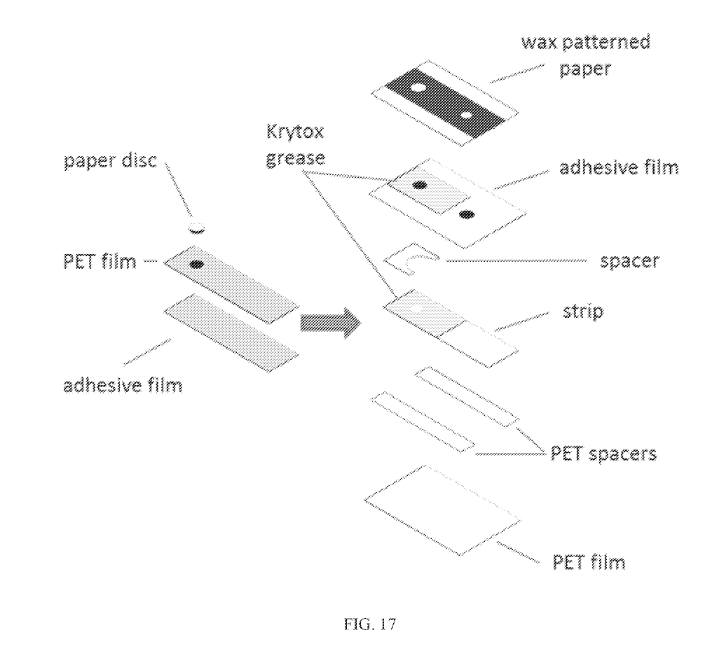

Experiments were performed to demonstrate that LAMP reactions could be successfully carried out in a sliding strip device using optimal materials. Sliding strip devices were fabricated utilizing Krytox grease to seal the top and low-tack PET film as a base (FIG. 17). A top layer consisting of wax patterned paper providing two hydrophilic stations was placed above a layer of adhesive film containing Krytox grease. These elements were situated above a spacer element and the sliding strip assembly comprising the test zone, also containing a layer of grease surrounding the test zone. Two PET film spacer elements were placed in parallel on opposite sides of the sliding member within the same plane such that the sliding member could slide along the length of these elements. The entire assembly was situated above a layer of PET film.

8 μL of LAMP reaction solution was transferred to the first port of the wax-patterned paper top layer and allowed to soak through to the reaction disk housed in the sliding strip. Once the disk was visibly wet (<1 min) the strip was slid to the zone between the first and second ports, forming a hermetic seal between the PET sliding strip spacer and the laminated wax-patterned paper top layer. The entire assembly was placed in a 65° C. oven for 1 hour to incubate. Upon removal from the oven, the sliding strip was pulled out of the device and returned to the oven for 5 min to dry the reaction disc. 4 μL of 0.1 mg/mL PI was added to the disc, and the fluorescence intensity was recorded at 616.95 nm.

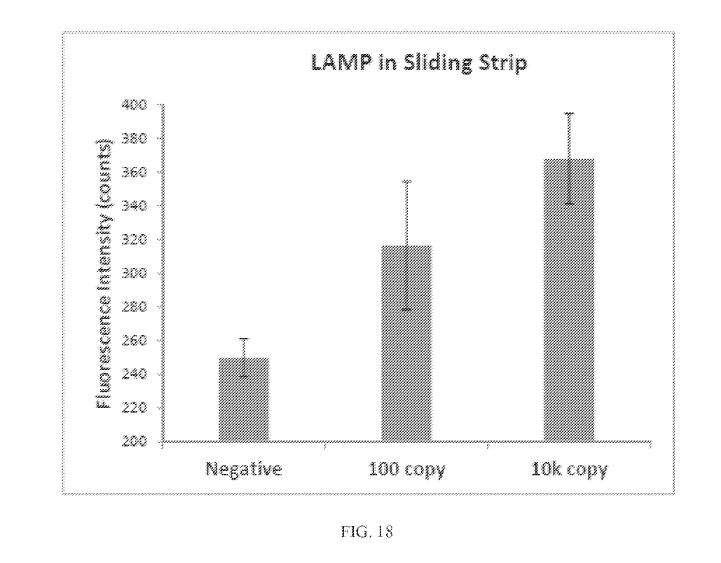

As confirmed by both fluorescence and gel electrophoresis, amplification detectable above background occurred for reaction discs containing as few as 100 copies of the genome (FIG. 18).

### Example 5

### Sample Preparation and Purification on a Sliding-Strip Device

Experiments were performed to optimize sample preparation and purification in a sliding strip device. The device architecture (FIG. 2) is similar to that of the device illustrated in FIG. 17 with the exceptions that no spacer sits above the sliding strip, and the device is not situated on top of a layer of PET film. Instead, the device incorporates as a base a layer of adhesive film resting on a piece of paper with the following features: exit apertures in the adhesive film to allow fluid to flow through the Whatman paper disc and wash channels formed using wax printing located on the bottom layer of paper (FIG. 2). The apertures and wash channels maintain fluidic communication with the disc in the sliding strip. The device works by the following steps: (i) a drop of sample is introduced into the top entry point on the device where it flows to the paper disc in the sliding strip layer, (ii) lysis chemistry present in the paper disc lyses the cells of the target organism while chemical treatments present on the paper disc simultaneously adsorb nucleic acids, (iii) the entire volume of sample is allowed to flow completely through the disc, in effect, concentrating the sample onto the disc, (iv) the disc is slid to the second region of the device where it encounters a drop of purification buffer which wicks through to a second wash channel and acts to wash away debris from the paper disc, and (v) the disc is slid to pick up LAMP reagents which selectively amplify the adsorbed target.

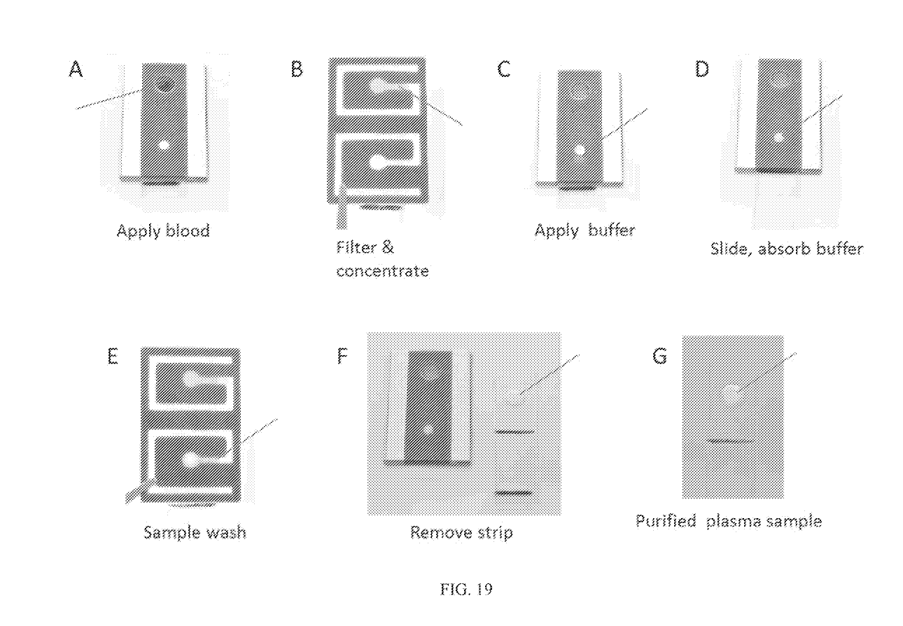

FIG. 19 illustrates each of the above conceptual steps as applied to an actual device. First, 30 μL of fingerstick whole blood was applied to the entry point where a plasma separation membrane was fixed (A). The plasma wicked through the paper disc in the strip to the wash channel found on the reverse side (B). A drop of PBS buffer was then applied in the second zone which remained static until the disc was slid into contact with it (C, D). The entire volume buffer was then absorbed through the disc which acts to purify unadsorbed components (E). The strip was then removed showing purified plasma on the paper disc (F, G).

### Environmental Stability

We have evaluated the environmental stability of many paper-based analysis devices under both room temperature and accelerated (45° C.) conditions. We have also evaluated the effects of humidity and temperature on the assay kinetics. Similar studies on working prototype devices are contemplated. Environmental effects will be quantified by measuring the resulting signal intensity from a given sample and comparing “aged” tests to control tests. Signal intensity can be measured by scanning a colorimetric result and quantitating the zone RGB value using software such as ImageJ. Various packaging approaches will also be explored using foil-lined bags, desiccant, vacuum sealing, etc. Target stability would be 1-2 year storage at room temperature.

### REFERENCES

References cited herein include:
1) http://www.hbvadvocate.org/hepatitis/hepB/measure_DNA.html.
2) Devries et al., J Clin Virol. 46 Suppl 4:S37-42, 2009.
3) Kleiber et al., Journal of Molecular Diagnostics. 2 (3) 158.
4) Hsiang et al., J Clin Microbiol. 48, 3539-43, 2010.
5) Whatman® Product Insert: “DNA Extraction from FTA® Cards Using the GenSolve DNA Recovery Kit.”
6) Bearinger, J.; Dugan, L.; Baker, B.; Hall, S.; Ebert, K.; Mioulet, V.; Madi, M.; and King, D. Development and Initial Results of a Low Cost, Disposable, Point-of-Care Testing Device for Pathogen Detection, IEEE transactions on Biomedical Engineering, 58, 805-808, 2010.
7) Hong, J.; Studer, V.; Hang, G.; Anderson, W.; and Quake, S. A nanoliter-scale nucleic acid processor with parallel architecture, Nature Biotechnology, 22, 435-439, 2004
8) Asiello, P.; and Baeumner, J.; Miniaturized Isothermal Nucleic Acid Amplification, a Review, Lab on a Chip, 11, 1420, 2011.
9) Weigl, B.; Domingo, G.; Gerlach, J.; Tang, D.; Harvey, D.; Talwar, N.; Fichtenholz, A.; van Lew, B.; and LaBarre, P. Non-instrumented Nucleic-Acid Amplification Assay, Proc. of SPIE, 6886, 688604, 2008.
10) Whitesides, et al. in international patent application WO 2009/121041.
11) Tang, W.; Chow, W.; Li, Y.; Kong, H.; Tang, Y.; and Lemieux, B. Nucleic Acid Assay System for Tier II Laboratories and Moderately Complex Clinics to Detect HIV in Low-Resource Settings, Journal of Infectious Disease, 201, S46-S51, 2010.
12) IVD Technology—“Point-of-care nucleic acid lateral-flow tests” http://www.ivdtechnology.com/article/point-care-nucleic-acid-lateral-flow-tests.
13) rapidSTRIPE Rickettsia assay—product insert, Westburg http://www.westburg.eu/en/site/products/molecular-diagnostics/pathogen-detection/per/rapidstripe-pathogen-detection-assays.
14) Aveyard, J.; Mehrabi, M.; Cossins, A.; Braven, H.; Wilson, R. One step visual detection of PCR products with gold nanoparticles and a nucleic acid lateral flow (NALF) device, Chemical Communications, 4251-4253, 2007.

This disclosure describes multiple aspects and embodiments of the invention. All combinations and permutations of the aspects and embodiments are contemplated. Further, throughout the description, where devices and compositions are described as having, including, or comprising specific components, or where processes and methods are described as having, including, or comprising specific steps, it is contemplated that, additionally, there are devices and compositions that consist essentially of, or consist of, the recited components, and that there are processes and methods according to the present invention that consist essentially of, or consist of, the recited processing steps.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A molecular diagnostic device for detecting a characteristic of a polynucleotide analyte present in a sample, the device comprising:
at least first, second, and third substantially planar members for collection and amplification of a sample, one or more of which comprises fluid-impermeable barriers which define boundaries of hydrophilic regions therein which support fluid flow therethrough by sorption, wicking or wetting, another of which defines a test zone for collection, amplification, and visualization of a sample;
at least one of said members being adapted for lateral movement relative to another to permit establishment of fluid flow communication serially between at least two said hydrophilic regions and said test zone, and to permit interaction between the test zone and reagents disposed in fluid communication with the test zone at least at first and second separate stations;
said first station comprising a polynucleotide capture region wherein said test zone is disposed in fluid communication with a sample inlet;
said second station comprising a polynucleotide amplification region wherein said test zone and captured polynucleotides are in fluid communication with a buffer inlet and amplification reagents.

2. The device of claim 1 wherein said first station comprises a single station or two or more substations, and in fluid communication with said test zone, two or more of: a cell membrane or protein coat lysing reagent, a filter for removing particulates, a polynucleotide restriction reagent for selectively restricting polynucleotide in a sample, a downstream liquid reservoir for drawing liquid from a said inlet to and through said test zone, and a surface which passively adsorbs polynucleotides, thereby to effect delivery to said second station of amplifiable polynucleotide, if analyte is present in said sample.

3. The device of claim 1 wherein said second station comprises a single station or two or more substations, and in fluid communication with said test zone, dried amplification reagents for conducting isothermal amplification.

4. The device of claim 3 wherein said second station further comprises a heater and thermal insulation for maintaining said amplification region at a predetermined elevated temperature, or an inlet to permit a wash of said test zone.

5. The device of claim 1 further comprising a site bounded by a seal for inhibiting evaporation from said test zone.

6. The device of claim 1 comprising a third station serving as an optically observable detection readout wherein said test zone and captured polynucleotides are in fluid communication with a buffer inlet and a dried polynucleotide detection reagent.

7. The device of claim 6 wherein said detection reagent functions to develop color in a said readout as an indication of the presence of amplified polynucleotide therein.

8. The device of claim 7 wherein said detection reagent at said third station comprises a labeled antibody reagent or a labeled nucleotide probe.

9. The device of claim 7 wherein said detection reagents at said third station comprise a colored particle conjugate.

10. The device of claim 8 wherein the antibody is labeled with an enzyme, a fluorophore, or a colored particle to permit colorimetric assessment of the presence of amplicons in said test zone.

11. The device of claim 6 comprising an enzyme substrate disposed in said device in flow communication with one of said hydrophilic regions.

12. The device of claim 1 comprising, an inlet for receiving a wash reagent, the inlet being in fluid communication with polynucleotides captured or amplified in said test zone, the washing reagent functioning to separate unbound species therein from said captured polynucleotides.

13. The device of claim 1 wherein establishment of fluid flow communication between a hydrophilic region at a station and captured oligonucleotides in said test zone is effected by movement of said members relative to each other to register vertically or horizontally said zone and a respective said hydrophilic region.

14. The device of claim 1 wherein said members comprise water adsorptive paper, cloth, or polymer film such as nitrocellulose or cellulose acetate.

15. The device of claim 1 wherein said fluid-impermeable barriers that define boundaries of said plural hydrophilic regions comprise barriers which penetrate the thickness of said member and comprise a photoresist, wax, poly(methylmethacrylate), an acrylate polymer, polystyrene, polyethylene, polyvinylchloride, a fluoropolymer, or a photo-polymerizable polymer that forms a hydrophobic polymer.

16. The device of claim 1 comprising said members disposed in parallel planes, and further comprising a fluid-impermeable layer disposed between adjacent said members and defining openings permitting fluid flow therethrough.

17. The device of claim 1 further comprising an adsorbent reservoir disposed in a said member downstream from said test zone for drawing fluid from or through a said hydrophilic region and through a said test zone.

18. The device of claim 1 comprising a plurality of test zones and corresponding plurality of said first and second stations, adapted for simultaneous testing for different analytes.

19. The device of claim 1 wherein the member comprising said test zone is adapted for removal from said device after said test zone is exposed to said second station, thereby to permit analysis of said test zone for the presence of amplicons by means separate from said device.

20. The device of claim 1 wherein said second station comprising dried, loop-mediated isothermal amplification reagents disposed in fluid communication with a buffer inlet.

21. The device of claim 2 wherein at least one of said cell membrane or protein coat lysing reagent and said polynucleotide restriction reagent are disposed in dried form upstream of or within said test zone.

22. The device of claim 1 wherein at least one of said amplification reagents are disposed in dried form upstream of said test zone at said second station.

23. The device of claim 1 further comprising a third station serving as an optically observable detection readout wherein said test zone and captured polynucleotides are in fluid communication with an inlet for receiving a solution of a polynucleotide detection reagent.

24. The device of claim 1 wherein each of said first and second stations comprises a wash inlet and a fluid flow path from said inlet to said test zone when said test zone is in registration with said flow path at said station.

25. The device of claim 1 further comprising a third station serving as an amplicons detection region comprising a fluid inlet and dried polynucleotide intercalating agent upstream of said test zone when said test zone is in registration with a flow path at said third station.

26. The device of claim 25 wherein the intercalating agent is propidium iodide.

27. An assay method comprising providing the device of claim 1, adding a sample to said sample inlet, moving one said member in relation to another to establish serially fluid communication between the test zone and said hydrophilic zones to permit fluid flow therebetween for a time interval and to execute multiple sequential steps of an assay designed to amplify a selected polynucleotide, if present in the sample, and examining said test zone or amplicons therein to determine the presence or absence of a said analyte.

28. The method of claim 27 wherein the test zone is examined in an analysis device separate from the device of claim 1.

29. An assay method comprising providing the device of claim 1, adding a sample to said inlet to capture oligonucleotides at said test zone, moving one said member in relation to another to establish fluid communication between the test zone containing captured oligonucleotides and amplification reagents to permit fluid flow therebetween for a time interval so as to amplify target oligonucleotide analyte, if present, and moving one said member in relation to another to establish fluid communication between the test zone containing captured and amplified oligonucleotides and a detection reagent to permit fluid flow therebetween for a time interval so as to optically detect the presence of amplicons, if present, in said test zone thereby to determine the presence or absence of a said analyte.

30. The method of claim 27 comprising the step of isothermally amplifying polynucleotide, if present in the sample.

31. The method of claim 27 comprising the step of removing the member comprising the test zone from said device and analyzing said test zone for the presence or absence of amplicons therein in a separate device.

32. The method of claim 27 comprising the step of removing matter from the test zone and analyzing said matter for the presence or absence of amplicons therein.
